(19) United States
(12) Patent Application Publication
(10) Pub. No.: US 2023/0201327 A1
(43) Pub. Date: Jun. 29, 2023

(54) RSV VACCINE BEARING ONE OR MORE P GENE MUTATIONS

(71) Applicant: The United States of America,as represented by the Secretary,Department of Health and Human Services, Bethesda, MD (US)

(72) Inventors: Peter L. Collins, Silver Spring, MD (US); Cyril Le Nouen, Bethesda, MD (US); Ursula J. Buchholz, Silver Spring, MD (US)

(73) Assignee: The United States of America,as represented by the Secretary,Department of Health and Human Services, Bethesda, MD (US)

(21) Appl. No.: 17/924,784

(22) PCT Filed: May 13, 2021
(86) PCT No.: PCT/US2021/032305

§ 371 Date: Nov. 11, 2022

Related U.S. Application Data
(60) Provisional application No. 63/023,949, filed on May 13, 2020.

(51) Int. Cl. A61K 39/12; A61P 37/04

(52) U.S. Cl. CPC A61K 39/12; A61P 37/04; A61K 2039/552

(57) ABSTRACT

Provided is a polynucleotide encoding a respiratory syncytial virus (RSV) variant having an attenuated phenotype comprising a modified RSV genome or antigenome that encodes a mutant RSV protein P that differs from a parental RSV protein P at one or more amino acid residues. In some embodiments, the polynucleotide is recombinant. The invention also relates to methods of vaccinating an animal with the RSV variant or a pharmaceutical composition containing the RSV variant or inducing an immune response by administering the RSV variant to an animal, and further relates to methods of producing an RSV variant vaccine. In some embodiments, the animal is a human.

### CROSS-REFERENCE TO PRIOR APPLICATION

This application claims benefit to U.S. Provisional Patent Application No. 63/023,949, filed May 13, 2020, which is hereby incorporated by reference in its entirety.

### STATEMENT REGARDING FEDERALLY SPONSORED RESEARCH OR DEVELOPMENT

Research supporting this application was carried out by the United States of America as represented by the Secretary, Department of Health and Human Services. This research was supported by the Intramural Research Program, Division of Intramural Research, NIAID of the National Institute of Health. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 179,715 Byte ASCII (Text) file named “754331_ST25.txt,” created on May 12, 2021.

### BACKGROUND OF THE INVENTION

Respiratory syncytial virus (RSV, also known as orthopneumovirus) belongs to the Pneumoviridae family of RNA viruses, and formerly belonged to the Paramyxoviridae family. RSV is an enveloped virus with a linear negative-sense RNA genome. Accordingly, the RNA genome is first transcribed before it is translated. The genome encodes 11 proteins, namely two non-structural proteins (NS1 and NS2), the RNA-binding nucleocapsid protein (N), the phosphoprotein (P), the internal matrix protein (M), the small hydrophobic surface glycoprotein (SH), the attachment glycoprotein (G), the fusion protein (F), two proteins encoded from the same mRNA (M2-1 and M2-2), and the large polymerase protein (L). The order of the open reading frames corresponding to these eleven proteins is 3′-NS1-NS2-N-P-M-SH-G-F-M2-1-M2-2-L-5′.

RSV is a widespread pathogen, known to cause respiratory tract infections which can lead to serious illness and even death, particularly in young children and older adults. RSV is estimated to have caused worldwide more than 33 million lower respiratory tract illnesses, three million hospitalizations, and nearly 200,000 childhood deaths annually, with many deaths occurring in developing countries. However, despite RSV's prevalence and the dangers associated with such infections, no RSV vaccine has been successfully developed to date. Accordingly, there is a need for RSV vaccines, such as those based on the disclosures herein.

### BRIEF SUMMARY OF THE INVENTION

The invention provides a polynucleotide encoding a respiratory syncytial virus (RSV) variant having an attenuated phenotype comprising a modified RSV genome or antigenome that encodes a mutant RSV protein P that differs from a parental RSV protein P at one or more amino acid residues. In some embodiments, the polynucleotide is recombinant. In some embodiments, at least one gene of the modified RSV genome or antigenome is codon pair deoptimized.

The invention also provides a RSV variant comprising a polynucleotide described herein, and a pharmaceutical composition comprising one or more of the RSV variants and at least one excipient. In some embodiments, two or more RSV variants are combined to form a multivalent RSV vaccine composition.

The invention further provides a method of vaccinating a subject, comprising administering a pharmaceutical composition as described herein to an animal, as well as a method of inducing an immune response in an animal, comprising administering one or more RSV variants described herein to an animal. In some embodiments, the animal is a human.

The invention still further provides a method of producing an RSV vaccine, comprising expressing one or more of the polynucleotides described herein in a cell.

Additional aspects and embodiments of the invention are as provided in the following detailed description.

### BRIEF DESCRIPTION OF THE DRAWINGS

FIG. 1 compares the structure of wild-type RSV to a codon-pair deoptimized (CPD) RSV variant (Min A).

FIG. 2 depicts a schematic of a protocol using incremental increases in temperature from 32 to 40° C. to apply temperature stress to the Min A RSV variant.

FIG. 3A illustrates the virus titer of Min A RSV variants at each temperature from 32 to 40° C. during the protocol depicted in FIG. 2.

FIG. 3B illustrates the virus titer of Min A RSV variants (controls) which were serially cultured at 32° C.

FIGS. 4A and 4B illustrate the point (passage 18) at which the Min A RSV variants depicted in FIGS. 3A and 3B, respectively, were sequenced using whole genome deep sequencing.

FIG. 5 depicts a chart summarizing mutations identified in the nucleotide sequence of the phosphoprotein (P) gene of the temperature stressed Min A RSV variants (each denoted with a lineage number), wherein each mutation causes a change in the amino acid sequence of the P protein (i.e., each mutation is a non-synonymous mutation).

FIG. 6 depicts virus titers in Vero cells (MOI=0.01 pfu/cell, 37° C.) infected with wild-type RSV or certain Min A RSV variants.

FIG. 7 depicts virus titers in Vero cells (MOI=3 pfu/cell, 37° C.) infected with wild-type RSV or certain Min A RSV variants.

FIG. 8 depicts a schematic for testing the replication and immunogenicity of certain Min A RSV variants in hamsters.

FIG. 9 depicts virus titers of certain Min A RSV variants, demonstrating the variants' replicative ability in hamsters.

FIG. 10 depicts levels of RSV-neutralizing antibodies in hamsters previously infected with certain Min A RSV variants.

FIG. 11 depict virus titers in hamsters previously challenged by certain Min A RSV variants in the nasal turbinates (NT) and in the lung.

FIGS. 12A-H are graphs depicting the growth kinetics (FIGS. 12A-12D) and plaque size (FIGS. 12E-12H) of certain RSV strains.

FIG. 13 is a set of graphs depicting fold increase in RNA synthesis rates of certain RSV strains as compared to the Min A RSV strain.

FIGS. 14A-F is a series of graphs and one image depicting protein expression of certain RSV strains (FIGS. 14A-14D) as well as depicting virus production of certain RSV strains (FIGS. 14E-14F).

FIGS. 15A-B are two graphs depicting the results of a temperature stress test of certain RSV strains (FIG. 15A) and the corresponding control (FIG. 15B).

FIGS. 16A-C are a gene map schematic of Min A and certain Min A derivatives (FIG. 16A) and graphs depicting the replication of those Min A derivatives (FIGS. 16B-16C).

FIGS. 17A-C are graphs depicting the replication (FIG. 17A), protective efficacy (FIG. 17B), and immunogenicity (FIG. 17C) of certain RSV strains.

FIGS. 18-A-B are two graphs depicting the results of a temperature stress test of certain RSV strains (FIG. 18A) and the corresponding control (FIG. 18B).

### DETAILED DESCRIPTION OF THE INVENTION

The invention provides a polynucleotide encoding a respiratory syncytial virus (RSV) variant having an attenuated phenotype comprising a modified RSV genome or antigenome that encodes a mutant RSV protein P that differs from a parental RSV protein P at one or more amino acid residues. In some embodiments, the polynucleotide is recombinant. In some embodiments, at least one gene of the modified RSV genome or antigenome is codon pair deoptimized.

### Codon Pair Deoptimization

In some embodiments, the genome or antigenome of the attenuated RSV variant is codon-pair deoptimized (CPD). CPD, along with codon deoptimization (CD) and increasing the dinucleotide CpG and UpA content, are techniques for modifying the nucleotide sequence of a virus that can lead to attenuation of the virus. In CD, the nucleotide sequence encoding a virus is modified to change one or more codons within an open reading frame (ORF) of a gene in a way that the amino acid encoded by the new codon is still the same as the amino acid encoded by the original codon, i.e., CD involves the insertion of synonymous mutations into ORFs. This process can affect certain characteristics of the nucleotide sequence, including codon bias, codon pair bias, CpG dinucleotide content, C+G content, density of deoptimized codons and deoptimized codon pairs, RNA secondary structure, translation frame sites, translation pause sites, the presence or absence of tissue specific microRNA recognition sequences, or any combination thereof.

CPD is based on the observation that certain codon pairs appear more or less frequently than expected. For example, the codon pair alanine-glutamate is encoded by the nucleotide bases GCC GAA and GCA GAG. If these codon pairs appeared randomly, then one would expect to see GCC GAA half of the time and GCA GAG half of the time. However, GCC GAA is strongly unrepresented, appearing only 1/7^{th} as often as GCA GAG. Without wishing to be bound to any particular theory, the existence of this codon pair bias is thought to stem from the effect certain codon pairs have on mRNA stability or synthesis, translation efficiency (some tRNA pairs interact less efficiently on the ribosome) and/or innate immunity (potentially a consequence of dinucleotide bias, insofar as the immune system seeks to suppress TLR ligands CpG and UpA).

Codon pair bias has been exploited to prepare weakened, i.e., attenuated, virus strains via CPD. See, e.g., U.S. Pat. No. 9,957,486, incorporated by reference in its entirety herein. With the advent of synthetic biology, including the increased availability and affordability of large-scale custom DNA synthesis, synonymous mutations to the nucleotide sequence of a virus's ORFs can be made in large numbers to take advantage of codon pair bias to attenuate the strain, by, e.g., reduce the replicative fitness of the resulting virus. In other words, CPD can now be applied on a genomic level. An advantage of using CPD as a technique for generating an attenuated RSV strain is that the probability of reversion to virulence is presumably extremely low when a large number of mutations are made in the strain. Following CPD, the nucleotide sequence containing the genome or antigenome of a CPD RSV variant encodes the same amino acid sequence as the genome or antigenome of a parental and/or wild-type RSV strain. However, other mutations can be introduced into the genome or antigenome of the CPD RSV variant, such that the genome or antigenome of the CPD RSV variant no longer encodes the same amino acid sequence as the genome or antigenome of the parental and/or wild-type RSV strain. Similarity on the amino acid level between a RSV variant and a parental and/or wild-type RSV strain is desirable because increased similarity between the sequences results in an increased likelihood that the CPD and parental and/or wild-type RSV strains will exhibit many or even all of the same epitopes. Inasmuch as cellular and humoral immunity are induced by such epitopes, CPD RSV variants desirably resemble parental and/or wild-type RSV strains on the amino acid level, at least in part.

Accordingly, in some embodiments, the inventive polynucleotide comprises a modified RSV genome or antigenome that is codon-pair deoptimized. In certain embodiments, the CPD RSV variant strain and the corresponding parental and/or wild-type strain encode the same amino acid sequence. However, identity at the amino acid level is not required. Thus, in other embodiments, the amino acid sequence encoded by the polynucleotide encoding the genome or antigenome of the CPD RSV variant is, or is at least, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% identical to the amino acid sequence encoded by the polynucleotide encoding the genome or antigenome of a wild-type RSV strain.

Nucleotide or amino acid sequence “identity,” as referenced herein, can be determined by comparing a nucleotide or amino acid sequence of interest to a reference nucleotide or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

In some embodiments, a computer program calculates the location and number of mutations within one more ORFs of an RSV genome or antigenome to generate a desired RSV CPD genome or antigenome nucleotide sequence. See, for example, Coleman et al., Science, 320(5884): 1784-1787 (2008). Such programs can generate under-represented codon pairs (i.e., deoptimize codon pairs) while leaving codon usage and nucleotide frequency unchanged.

Accordingly, in some embodiments, the codon usage and/or nucleotide frequency in one or more ORFs in the genome or antigenome of a RSV variant is the same as the codon usage and/or nucleotide frequency in the corresponding one or more ORFs in the genome or antigenome of a parental and/or wild-type RSV strain. In other embodiments, the codon usage and/or nucleotide frequency in one or more ORFs in the genome of a RSV variant is different than the codon usage and/or nucleotide frequency in the corresponding one or more ORFs in the genome or antigenome of a parental and/or wild-type RSV strain. In some embodiments, the codon usage and/or nucleotide frequency in all ORFs in the genome or antigenome of a RSV variant is about the same as in all ORFs in the genome or antigenome of a parental and/or wild-type RSV strain. In a preferred embodiment, the codon usage and/or nucleotide frequency of the ORFs in the genome of a RSV variant coding for RSV proteins NS1, NS2, N, P, M. and SH is about the same as in the corresponding ORFs in the genome or antigenome of a parental and/or wild-type RSV strain.

Moreover, using CPD, the level of attenuation of the virus can be modulated to a desirable level by adjusting the number of mutations introduced into the nucleotide sequence encoding one or more ORFs of the viral proteins. Accordingly, in some embodiments, the polynucleotide comprising the genome or antigenome of the CPD RSV variant contains 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, or 2700 synonymous mutations, or synonymous mutations in a range bounded by any two of the foregoing values, in comparison to the genomic or antigenomic sequence of a parental and/or wild-type RSV strain. In certain embodiments, the polynucleotide comprising the genome or antigenome of the CPD RSV variant is recombinant, isolated, and/or not naturally occurring, i.e., not found in nature.

In some embodiments, the mutations described herein, when used either alone or in combination with another mutation, may provide for different levels of virus attenuation, providing the ability to adjust the balance between attenuation and immunogenicity, and provide a more stable genotype than that of the parental virus.

The level of attenuation of vaccine virus may be determined by, for example, quantifying the amount of virus present in the respiratory tract of an immunized host and comparing the amount to that produced by parental and/or wild-type RSV or other attenuated RSV viruses which have been evaluated as candidate vaccine strains. For example, the attenuated virus of the invention will have a greater degree of restriction of replication in the upper respiratory tract of a highly susceptible host, such as a chimpanzee, compared to the levels of replication of parental and/or wild-type virus, e.g., 10- to 1000-fold less. In order to further reduce the development of rhinorrhea, which is associated with the replication of virus in the upper respiratory tract, an ideal vaccine candidate virus should exhibit a restricted level of replication in both the upper and lower respiratory tract. The RSV variant disclosed herein, to be effective, should be sufficiently infectious and immunogenic in humans to confer protection in vaccinated individuals. Methods for determining levels of RSV in the nasopharynx of an infected host are well known in the literature. Specimens are obtained by aspiration or washing out of nasopharyngeal secretions and virus quantified in tissue culture or other by laboratory procedure. See, for example, Belshe et al., J. Med. Virology, 1: 157-162 (1977), Friedewald et al., J. Amer. Med. Assoc., 204: 690-694 (1968); Gharpure et al., J. Virol., 3: 414-421 (1969); and Wright et al., Arch. Ges. Virusforsch., 41: 238-247 (1973). The virus can conveniently be measured in the nasopharynx of host animals, such as chimpanzees.

In some embodiments, the RSV variant may comprise other known attenuating mutations of RSV and/or related viruses to yield other attenuation phenotypes. A number of such mutations are known in the art. For instance, in some embodiments, the M2-2 ORF, the NS1 ORF or the NS2 ORF may be partially or completely deleted from the CPD RSV genome or antigenome.

In some embodiments, the inventive polynucleotide which encodes a recombinant respiratory syncytial virus (RSV) variant having an attenuated phenotype comprises a modified RSV genome or antigenome that encodes a mutant RSV protein P that differs from a parental RSV protein P at one or more amino acid residues, wherein the nucleotide sequence of the modified RSV genome or antigenome encoding one or more of RSV proteins NS1, NS2, N, P, M, and SH has about 70% to about 95% identity with the nucleotide sequence of a parental and/or wild-type RSV genome or antigenome encoding the same one or more of RSV proteins NS1, NS2, N, P, M, and SH. In some embodiments, the nucleotide sequence of the modified RSV genome or antigenome encoding one or more of RSV proteins NS1, NS2, N, P, M, and SH is CPD.

In some embodiments, the polynucleotide comprising the nucleotide sequence of the CPD RSV genome or antigenome encoding one or more of RSV proteins NS1, NS2, N, P, M, SH, G, F, M2-1, M2-2, and L has at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 percent identity with a nucleotide sequence of a parental and/or wild-type RSV genome encoding the same one or more of RSV proteins NS1, NS2, N, P, M, SH, G, F, M2-1, M2-2, and L. In some embodiments, the polynucleotide comprising the nucleotide sequence of the CPD RSV genome or antigenome encoding one or more of RSV proteins NS1, NS2, N, P, M, and SH has at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 percent identity with a nucleotide sequence of a parental and/or wild-type RSV genome encoding the same one or more of RSV proteins NS1, NS2, N, P, M, and SH. In some embodiments, the parental and/or wild-type RSV genome is represented by SEQ ID NO: 1.

In some embodiments, one or more ORFs of the modified RSV genome or antigenome are CPD. The one or more ORFs that have been codon pair deoptimized (i.e., the nucleotide sequence of the modified RSV genome or antigenome encoding one or more of RSV proteins) can be selected from the group consisting of the ORFs that encode an RSV protein, namely the structural protein NS1 or NS2, the RNA-binding nucleocapsid protein (N), the phosphoprotein (P), the internal matrix protein (M), the small hydrophobic surface glycoprotein (SH), the attachment glycoprotein (G), the fusion protein (F), one of two proteins encoded by portions of the same mRNA (M2-1 and M2-2), and the large polymerase protein (L). This includes any combination of the RSV proteins. The one or more ORFs that have been codon pair deoptimized can include 1, 2, 3, 4, 5, 6, 7, 8, 9, 10, or 11 ORFs. In certain embodiments, the one or more ORFs that have been codon pair deoptimized encode only NS1, only NS2, only N, only P, only M, or only SH. In certain other embodiments, the one or more ORFs that have been codon pair deoptimized encode NS1 and NS2, or NS1 and N, or NS1 and P, or NS1 and M, or NS1 and SH, or NS1, NS2, and N, or NS1, NS2, and P, or NS1, NS2, and M, or NS1, NS2, and SH, or NS1, NS2, N, and P, or NS1, NS2, N, and M, or NS1, NS2, N, and SH, or NS1, N, and P, or NS1, N, and P, or NS1, N, and M, NS1, N, and SH, or NS1, P, and M, or NS1, P, and SH, or NS1, M, and SH, or NS1, NS2, N, and P, or NS1, NS2, N, and M, or NS1, NS2, N, and SH, or NS1, NS2, P, and M, or NS1, NS2, P, and SH, or NS1, NS2, N, P, and M, or NS1, NS2, N, P, and SH, or NS2 and N, or NS2 and P, or NS2 and M, or NS2 and SH, or NS2, N, and P, or NS2, N, and M, or NS2, N, and SH, or NS2, P, and M, or NS2, P, and SH, or NS2, N, P, and M, or NS2, N, P, and SH, or NS2, N, P, M, and SH, or N and P, or N and M, or N and SH, or N, P, and M, or N, P, and SH, or N, P, M, and SH, or P and M, or P and SH, or P, M, and SH, or M and SH. In a preferred embodiment, the one or more ORFs that have been codon pair deoptimized encode NS1, NS2, N, P, M, and SH. In such embodiments, the remaining ORFs that are not specifically indicated as codon pair deoptimized are not codon pair deoptimized.

In certain embodiments, the one or more ORFs of the modified RSV genome or antigenome that have been codon pair deoptimized each independently have a codon-pair-bias value of less than about 0.0. In another embodiment, the one or more ORFs that have been codon pair deoptimized each independently have a codon-pair-bias value of less than about 0.0. In certain embodiments, the one or more ORFs that have been codon pair deoptimized each independently have a codon-pair-bias value of less than about −0.10. In certain embodiments, the one or more ORFs that have been codon pair deoptimized each independently have a codon-pair-bias value of between about −0.10 to −0.40. In further embodiments, the one or more ORFs that have been codon pair deoptimized are selected from NS1, NS2, N, P, M and SH, wherein the codon-pair-bias values of NS1, NS2, N, P, M and SH are respectively about −0.14 (NS1), about −0.22 (NS2), about −0.31 (N), about −0.24 (P), −0.31 (M), and −0.18 (SH). In a further embodiment, the one or more ORFs that have been codon pair deoptimized are NS1, NS2, N, P, M and SH, for a total of six codon pair deoptimized ORFs, wherein the codon-pair-bias values of NS1, NS2, N, P, M and SH are respectively about −0.14 (NS1), about −0.22 (NS2), about −0.31 (N), about −0.24 (P), −0.31 (M), and −0.18 (SH). Codon pair-bias values are calculated according to the algorithms set forth in Coleman et al., Science, 320(5884): 1784-1787 (2008).

In certain embodiments, the ORF, i.e., nucleotide sequence, encoding the NS1 protein in the genome or antigenome of the RSV variant is codon pair deoptimized. In certain embodiments, the nucleotide sequence of the modified RSV genome encoding RSV protein NS1 has about 75% to about 95% identity with the ORF, i.e., nucleotide sequence, of the parental and/or wild-type RSV genome encoding RSV protein NS1. In further embodiments, the nucleotide sequence of the modified RSV genome encoding RSV NS1 protein has about 87% identity with the nucleotide sequence of the parental and/or wild-type RSV genome encoding RSV protein NS1. In other embodiments, the nucleotide sequence of the modified RSV genome encoding RSV NS1 protein is represented by nucleotides 99 to 518 of SEQ ID NO: 2.

In certain embodiments, the ORF, i.e., nucleotide sequence, encoding the NS2 protein in the genome or antigenome of the RSV variant is codon pair deoptimized. In certain embodiments, the nucleotide sequence of the modified RSV genome encoding RSV protein NS2 has about 75% to about 95% identity with the ORF, i.e., nucleotide sequence, of the parental and/or wild-type RSV genome encoding RSV protein NS2. In further embodiments, the nucleotide sequence of the modified RSV genome encoding RSV NS2 protein has about 88% identity with the nucleotide sequence of the parental and/or wild-type RSV genome encoding RSV protein NS2. In other embodiments, the nucleotide sequence of the modified RSV genome encoding RSV NS2 protein is represented by nucleotides 628 to 1002 of SEQ ID NO: 2.

In certain embodiments, the ORF, i.e., nucleotide sequence, encoding the N protein in the genome or antigenome of the RSV variant is CPD. In certain embodiments, the nucleotide sequence of the modified RSV genome encoding RSV protein N has about 70% to about 90% identity with the ORF, i.e., nucleotide sequence, of the parental and/or wild-type RSV genome encoding RSV protein N. In certain embodiments, the nucleotide sequence of the modified RSV genome encoding RSV N protein has about 80% identity with the nucleotide sequence of the parental and/or wild-type RSV genome encoding RSV protein N. In further embodiments, the nucleotide sequence of the modified RSV genome encoding RSV N protein is represented by nucleotides 1141 to 2316 of SEQ ID NO: 2.

In certain embodiments, the ORF, i.e., nucleotide sequence, encoding the P protein in the genome or antigenome of the RSV variant is codon pair deoptimized. In certain embodiments, the nucleotide sequence of the modified RSV genome encoding RSV protein P has about 75% to about 95% identity with the ORF, i.e., nucleotide sequence, of the parental and/or wild-type RSV genome encoding RSV protein P. In certain embodiments, the nucleotide sequence of the modified RSV genome encoding RSV NS1 protein has about 84% identity with the nucleotide sequence of the parental and/or wild-type RSV genome encoding RSV protein P. In further embodiments, the nucleotide sequence of the modified RSV genome encoding RSV P protein is represented by nucleotides 2347 to 3072 of SEQ ID NO: 2.

In certain embodiments, the ORF, i.e., nucleotide sequence, encoding the M protein in the genome or antigenome of the RSV variant is codon pair deoptimized. In certain embodiments, the nucleotide sequence of the modified RSV genome encoding RSV protein M has about 75% to about 95% identity with the ORF, i.e., nucleotide sequence, of the parental and/or wild-type RSV genome encoding RSV protein M. In certain embodiments, the nucleotide sequence of the modified RSV genome encoding RSV M protein has about 83% identity with the nucleotide sequence of the parental and/or wild-type RSV genome encoding RSV protein M. In further embodiments, the nucleotide sequence of the modified RSV genome encoding RSV M protein is represented by nucleotides 3262 to 4032 of SEQ ID NO: 2.

In certain embodiments, the ORF, i.e., nucleotide sequence, encoding the SH protein in the genome or antigenome of the RSV variant is codon pair deoptimized. In certain embodiments, the nucleotide sequence of the modified RSV genome encoding RSV protein SH has about 85% to about 95% identity with the ORF, i.e., nucleotide sequence, of the parental and/or wild-type RSV genome encoding RSV protein SH. In certain embodiments, the nucleotide sequence of the modified RSV genome encoding RSV SH protein has about 92% identity with the nucleotide sequence of the parental and/or wild-type RSV genome encoding RSV protein SH. In further embodiments, the nucleotide sequence of the modified RSV genome encoding RSV SH protein is represented by nucleotides 4304 to 4498 of SEQ ID NO: 2.

In an embodiment, the one or more ORFs that have been codon pair deoptimized encode NS1, NS2, N, P, M, and SH, and the nucleotide sequence represented by SEQ ID NO: 2 contains the nucleotide sequence of the codon pair deoptimized ORFs.

In certain embodiments, an amino acid sequence of the one or more of RSV proteins NS1, NS2, N, P, M and SH encoded by the nucleotide sequence of the modified RSV genome or antigenome is identical to an amino acid sequence of the same one or more of RSV proteins NS1, NS2, N, P, M and SH encoded by the nucleotide sequence of the parental and/or wild-type RSV genome or antigenome, except at the one or more amino acid residues where the mutant RSV protein P differs from the parental and/or wild-type RSV protein P. In certain embodiments, the amino acid sequence of the one or more of RSV proteins NS1, NS2, N, P, M and SH encoded by the nucleotide sequence of the modified RSV genome or antigenome is, or is at least, 90%, 91%, 92%, 93%, 94%, 95%, 96%, 97%, 98%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, or 99.9% identical to an amino acid sequence of the same one or more of RSV proteins NS1, NS2, N, P, M and SH encoded by the nucleotide sequence of the parental and/or wild-type RSV genome or antigenome. In some embodiments, when the amino acid sequence encoded by the nucleotide sequence of the modified RSV genome or antigenome encodes two or more RSV proteins, percent identity is calculated using the combined amino acid sequence of the two or more RSV proteins. In other embodiments, it is calculated for each of the two or more RSV proteins individually, and the percent identity for each protein must be at least the recited percent identity.

As stated previously, the probability of reversion to virulence (i.e., de-attenuation) is presumed to be low when a large number of mutations is made in a RSV variant, such as a CPD RSV variant. However, in order to study de-attenuation, it is advantageous to subject the virus of interest to strong selective pressure. A limitation of previous de-attenuation studies of other CPD viruses was a lack of such strong selective pressure. See, e.g., Bull et al., Mol. Biol. and Evol., 29(10): 2997-3004 (2012); Burns et al., J. Virol., 80(19): 9687-9696 (2006); Cheng et al., Virology, 501: 35-46 (2015); Coleman et al., Science, 320(5884): 1784-7 (2008); Meng et al., MBio, 5.5: 301704-14 (2014); Mueller et al., J. Virol., 80(19): 9687-96 (2006), Ni et al., Virology, 450: 132-139 (2014); Nougairede et al., PLoS Pathogens 9.2 (2013). Given that at least some CPD RSV variants are temperature sensitive, such RSV variants provide an excellent subject for studying de-attenuation of CPD viruses. See, e.g., U.S. Patent Application Publication 2019/0233476 A1, incorporated by reference in its entirety herein. Temperature sensitive viruses have a shut-off temperature, at which they fail to continue replicating.

By serially culturing such CPD RSV viruses in vitro while exposing them to step-wise increases in temperature from a permissive temperature, i.e., a temperature at which the virus can continue to replicate, to temperatures approaching and reaching the viruses' shut-off temperatures, mutations can be identified that rescue replication in those CPD RSV viruses near or at their previous shut-off temperatures.

Using this technique, the inventors identified mutations in RSV protein P that rescued replication in certain CPD RSV strains. When these de-attenuating mutations were introduced back into the original CPD RSV strains, it was surprisingly found that the resulting RSV variants exhibited increased attenuation, increased genetic stability, and/or increased immunogenicity in comparison to the original CPD RSV strains that did not contain any of the presumably de-attenuating mutations.

### Polynucleotide of RSV Variant Having an Attenuated Phenotype

The invention includes a polynucleotide encoding a respiratory syncytial virus (RSV) variant having an attenuated phenotype comprising a modified RSV genome or antigenome that encodes a mutant RSV P protein that differs from a parental and/or wild-type RSV P protein at one or more amino acid residues. The polynucleotide comprises a genome or antigenome that encodes, at least in part, the amino acid sequence comprising the RSV protein phosphoprotein (P). The amino acid sequence of the P protein encoded by the genome or antigenome of the polynucleotide comprises one or more mutations when compared to the amino acid sequence of an RSV P protein in a parental and/or wild-type RSV. In some embodiments, the polynucleotide is recombinant. In some embodiments, the polynucleotide is isolated. Preferably, the polynucleotide is not naturally occurring, i.e., not found in nature. In some embodiments, at least one gene of the modified RSV genome or antigenome having an attenuated phenotype is CPD.

In certain embodiments, the modified RSV genome or antigenome encodes a mutant RSV P protein that differs from a parental and/or wild-type RSV P protein at 1, 2, 3, 4, 5, 6, 7, 8, 9, 10, 11, 12, 13, 14, 15, 16, 17, 18, 19, or 20 amino acid residues. In some embodiments, the modified RSV genome or antigenome encodes a mutant RSV P protein that has an amino acid sequence that differs from the amino acid sequence set forth in SEQ ID NO: 6 at one or more positions selected from the group consisting of 19-34, 107, 229, 234, and 235. This includes any and all combinations of mutations at these positions. Preferably the one or more positions are selected from 25, 26, 27, 28, 29, 30, 31, 32, 33, 34, and any combination thereof. More preferably, the one or more positions are selected from 25, 27, 28, 32, 34, and any combination thereof. In some embodiments, the one or more positions are 32 and 34. In other embodiments, the one or more positions are 27 and 28.

In certain embodiments, the polynucleotide comprising a modified RSV genome or antigenome encodes a mutant RSV P protein with an amino acid sequence that differs from the amino acid sequence set forth in SEQ ID NO: 6 at at least position 19. In certain embodiments, the residue at position 19 of the amino acid sequence is isoleucine. In a further embodiment, the polynucleotide comprising a modified RSV genome or antigenome encodes a mutant RSV P protein with an amino acid sequence that differs from the amino acid sequence set forth in SEQ ID NO: 6 only at position 19, wherein the residue at position 19 of the amino acid sequence is isoleucine, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 19, wherein the residue at position 19 of the amino acid sequence is isoleucine, and wherein the NS1, NS2, N, P, M, and SH genes of the modified RSV genome or antigenome are each CPD.

In certain embodiments, the polynucleotide comprising a modified RSV genome or antigenome encodes a mutant RSV P protein with an amino acid sequence that differs from the amino acid sequence set forth in SEQ ID NO: 6 at at least position 20. In certain embodiments, the residue at position 20 of the amino acid sequence is tyrosine. In a further embodiment, the polynucleotide comprising a modified RSV genome or antigenome encodes a mutant RSV P protein with an amino acid sequence that differs from the amino acid sequence set forth in SEQ ID NO: 6 only at position 20, wherein the residue at position 20 of the amino acid sequence is tyrosine, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 20, wherein the residue at position 20 of the amino acid sequence is tyrosine, and wherein the NS1, NS2, N, P, M, and SH genes of the modified RSV genome or antigenome are each CPD.

In certain embodiments, the polynucleotide comprising a modified RSV genome or antigenome encodes a mutant RSV P protein with an amino acid sequence that differs from the amino acid sequence set forth in SEQ ID NO: 6 at at least position 25. In certain embodiments, the residue at position 25 of the amino acid sequence is threonine, glutamic acid, or asparagine. In another embodiment, the residue at position 25 of the amino acid sequence is threonine.

In a further embodiment, the polynucleotide comprising a modified RSV genome or antigenome encodes a mutant RSV P protein with an amino acid sequence that differs from the amino acid sequence set forth in SEQ ID NO: 6 only at position 25, wherein the residue at position 25 of the amino acid sequence is threonine, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 25, wherein the residue at position 25 of the amino acid sequence is threonine, and wherein the NS1, NS2, N, P, M, and SH genes of the modified RSV genome or antigenome are each CPD. In another embodiment, the residue at position 25 of the amino acid sequence is threonine. In a further embodiment, the polynucleotide comprising a modified RSV genome or antigenome encodes a mutant RSV P protein with an amino acid sequence that differs from the amino acid sequence set forth in SEQ ID NO: 6 only at position 25, wherein the residue at position 25 of the amino acid sequence is asparagine, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 25, wherein the residue at position 25 of the amino acid sequence is asparagine, and wherein the NS1, NS2, N, P, M, and SH genes of the modified RSV genome or antigenome are each CPD.

In a further embodiment, the polynucleotide comprising a modified RSV genome or antigenome encodes a mutant RSV P protein with an amino acid sequence that differs from the amino acid sequence set forth in SEQ ID NO: 6 only at position 25, wherein the residue at position 25 of the amino acid sequence is glutamic acid, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 25, wherein the residue at position 25 of the amino acid sequence is glutamic acid, and wherein the NS1, NS2, N, P, M, and SH genes of the modified RSV genome or antigenome are each CPD.

In certain embodiments, the polynucleotide comprising a modified RSV genome or antigenome encodes a mutant RSV P protein with an amino acid sequence that differs from the amino acid sequence set forth in SEQ ID NO: 6 at at least position 27. In certain embodiments, the residue at position 27 of the amino acid sequence is glutamic acid or asparagine. In another embodiment, the residue at position 27 of the amino acid sequence is asparagine. In a further embodiment, the polynucleotide comprising a modified RSV genome or antigenome encodes a mutant RSV P protein with an amino acid sequence that differs from the amino acid sequence set forth in SEQ ID NO: 6 only at position 27, wherein the residue at position 27 of the amino acid sequence is asparagine, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 27, wherein the residue at position 27 of the amino acid sequence is asparagine, and wherein the NS1, NS2, N, P, M, and SH genes of the modified RSV genome or antigenome are each CPD. In a further embodiment, the modified RSV genome or antigenome comprises, consists essentially of, or consists of, nucleotide sequence SEQ ID NO: 20. In another embodiment, the polynucleotide comprises, consists essentially of, or consists of, nucleotide sequence SEQ ID NO: 20.

In another embodiment, the polynucleotide comprising a modified RSV genome or antigenome encodes (a) a mutant RSV P protein with an amino acid sequence that differs from the amino acid sequence set forth in SEQ ID NO: 6 only at position 27, wherein the residue at position 27 of the amino acid sequence is asparagine, and (b) a mutant L protein that differs from the amino acid sequence set forth in SEQ ID NO: 13 only at position 151, wherein the residue at position 151 is alanine; wherein the modified RSV genome or antigenome comprises nucleotide sequence SEQ ID NO: 17 corresponding to an N 5′ untranslated region (UTR); and wherein the NS1, NS2, N, P, M, and SH genes of the modified RSV genome or antigenome are each CPD. In a further embodiment, the modified RSV genome or antigenome comprises, consists essentially of, or consists of, nucleotide sequence SEQ ID NO: 21. In another embodiment, the polynucleotide comprises, consists essentially of, or consists of, nucleotide sequence SEQ ID NO: 21.

In a further embodiment, the polynucleotide comprising a modified RSV genome or antigenome encodes a mutant RSV P protein with an amino acid sequence that differs from the amino acid sequence set forth in SEQ ID NO: 6 only at position 27, wherein the residue at position 27 of the amino acid sequence is glutamic acid, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 27, wherein the residue at position 27 of the amino acid sequence is glutamic acid, and wherein the NS1, NS2, N, P, M, and SH genes of the modified RSV genome or antigenome are each CPD.

In certain embodiments, the polynucleotide comprising a modified RSV genome or antigenome encodes a mutant RSV P protein with an amino acid sequence that differs from the amino acid sequence set forth in SEQ ID NO: 6 at at least position 28. In certain embodiments, the residue at position 28 of the amino acid sequence is valine, isoleucine, leucine, proline, or serine. In another embodiment, the residue at position 28 of the amino acid sequence is valine. In a further embodiment, the polynucleotide comprising a modified RSV genome or antigenome encodes a mutant RSV P protein with an amino acid sequence that differs from the amino acid sequence set forth in SEQ ID NO: 6 only at position 28, wherein the residue at position 28 of the amino acid sequence is valine. In another embodiment, the polynucleotide comprising a modified RSV genome or antigenome encodes a mutant RSV P protein with an amino acid sequence that differs from the amino acid sequence set forth in SEQ ID NO: 6 only at position 28, wherein the residue at position 28 of the amino acid sequence is valine, and wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 28, wherein the residue at position 28 of the amino acid sequence is valine, and wherein the NS1, NS2, N, P, M, and SH genes of the modified RSV genome or antigenome are each CPD. In a further embodiment, the modified RSV genome or antigenome comprises, consists essentially of, or consists of, nucleotide sequence SEQ ID NO: 22. In another embodiment, the polynucleotide comprises, consists essentially of, or consists of, nucleotide sequence SEQ ID NO: 22.

In another embodiment, the polynucleotide comprising a modified RSV genome or antigenome encodes (a) a mutant RSV P protein with an amino acid sequence that differs from the amino acid sequence set forth in SEQ ID NO: 6 only at position 28, wherein the residue at position 28 of the amino acid sequence is valine, (b) a mutant L protein that differs from the amino acid sequence set forth in SEQ ID NO: 13 only at position 2084, wherein the residue at position 2084 is proline, and (c) a mutant M protein that differs from the amino acid sequence set forth in SEQ ID NO: 7 only at position 123, wherein the residue at position 2084 is methionine, and wherein the NS1, NS2, N, P, M, and SH genes of the modified RSV genome or antigenome are each CPD. In a further embodiment, the modified RSV genome or antigenome comprises, consists essentially of, or consists of, nucleotide sequence SEQ ID NO: 23. In another embodiment, the polynucleotide comprises, consists essentially of, or consists of, nucleotide sequence SEQ ID NO: 23

In another embodiment, the polynucleotide comprising a modified RSV genome or antigenome encodes (a) a mutant RSV P protein with an amino acid sequence that differs from the amino acid sequence set forth in SEQ ID NO: 6 only at position 28, wherein the residue at position 28 of the amino acid sequence is valine, (b) a mutant L protein that differs from the amino acid sequence set forth in SEQ ID NO: 13 only at position 2084, wherein the residue at position 2084 is proline, and (c) a mutant M protein that differs from the amino acid sequence set forth in SEQ ID NO: 7 only at position 123, wherein the residue at position 2084 is methionine; wherein the modified RSV genome or antigenome comprises (a′) nucleotide sequence SEQ ID NO: 16 corresponding to an NS2 5′ untranslated region (UTR), (b′) nucleotide sequence SEQ ID NO: 18 corresponding to P gene start and 5′ UTR regions, and (c′) nucleotide sequence SEQ ID NO: 19 corresponding to a P 3′ UTR; and wherein the NS1, NS2, N, P, M, and SH genes of the modified RSV genome or antigenome are each CPD. In a further embodiment, the modified RSV genome or antigenome comprises, consists essentially of, or consists of, nucleotide sequence SEQ ID NO: 24. In another embodiment, the polynucleotide comprises, consists essentially of, or consists of, nucleotide sequence SEQ ID NO: 24.

In another embodiment, the residue at position 28 of the amino acid sequence is valine. In a further embodiment, the polynucleotide comprising a modified RSV genome or antigenome encodes a mutant RSV P protein with an amino acid sequence that differs from the amino acid sequence set forth in SEQ ID NO: 6 only at position 28, wherein the residue at position 28 of the amino acid sequence is proline, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 28, wherein the residue at position 28 of the amino acid sequence is proline, and wherein the NS1, NS2, N, P, M, and SH genes of the modified RSV genome or antigenome are each CPD.

In another embodiment, the residue at position 28 of the amino acid sequence is valine. In a further embodiment, the polynucleotide comprising a modified RSV genome or antigenome encodes a mutant RSV P protein with an amino acid sequence that differs from the amino acid sequence set forth in SEQ ID NO: 6 only at position 28, wherein the residue at position 28 of the amino acid sequence is isoleucine, and preferably wherein the modified RSV genome or antigenome comprises at least one gene that is CPD. In a further embodiment, the polynucleotide comprising a modified RSV genome or antigenome encodes a mutant RSV P protein with an amino acid sequence that differs from the amino acid sequence set forth in SEQ ID NO: 6 only at position 28, wherein the residue at position 28 of the amino acid sequence is isoleucine, and wherein the NS1, NS2, N, P, M, and SH genes of the modified RSV genome or antigenome are each CPD.

In certain embodiments, the polynucleotide comprising a modified RSV genome or antigenome encodes a mutant RSV P protein with an amino acid sequence that differs from the amino acid sequence set forth in SEQ ID NO: 6 at at least position 32. In certain embodiments, the residue at position 32 of the amino acid sequence is threonine. In a further embodiment, the polynucleotide comprising a modified RSV genome or antigenome encodes a mutant RSV P protein with an amino acid sequence that differs from the amino acid sequence set forth in SEQ ID NO: 6 only at position 32, wherein the residue at position 32 of the amino acid sequence is threonine, and preferably wherein the modified RSV genome or antigenome comprises at least one gene that is CPD. In a further embodiment, the polynucleotide comprising a modified RSV genome or antigenome encodes a mutant RSV protein with an amino acid sequence that differs from the amino acid sequence set forth in SEQ ID NO: 6 only at position 32, wherein the residue at position 32 of the amino acid sequence is threonine, and wherein the NS1, NS2, N, P, M, and SH genes of the modified RSV genome or antigenome are each CPD.

In certain embodiments, the polynucleotide comprising a modified RSV genome or antigenome encodes a mutant RSV protein P with an amino acid sequence that differs from the amino acid sequence set forth in SEQ ID NO: 6 at at least position 34. In certain embodiments, the residue at position 34 of the amino acid sequence is serine. In a further embodiment, the polynucleotide comprising a modified RSV genome or antigenome encodes a mutant RSV protein with an amino acid sequence that differs from the amino acid sequence set forth in SEQ ID NO: 6 only at position 34, wherein the residue at position 34 of the amino acid sequence is serine, and preferably wherein the modified RSV genome or antigenome comprises at least one gene that is CPD. In a further embodiment, the polynucleotide comprising a modified RSV genome or antigenome encodes a mutant RSV protein with an amino acid sequence that differs from the amino acid sequence set forth in SEQ ID NO: 6 only at position 34, wherein the residue at position 34 of the amino acid sequence is serine, and wherein the NS1, NS2, N, P, M, and SH genes of the modified RSV genome or antigenome are each CPD.

In certain embodiments, the polynucleotide comprising a modified RSV genome or antigenome encodes a mutant RSV protein P with an amino acid sequence that differs from the amino acid sequence set forth in SEQ ID NO: 6 at at least position 107. In certain embodiments, the residue at position 107 of the amino acid sequence is lysine. In a further embodiment, the polynucleotide comprising a modified RSV genome or antigenome encodes a mutant RSV protein with an amino acid sequence that differs from the amino acid sequence set forth in SEQ ID NO: 6 only at position 107, wherein the residue at position 107 of the amino acid sequence is lysine, and preferably wherein the modified RSV genome or antigenome comprises at least one gene that is CPD. In a further embodiment, the polynucleotide comprising a modified RSV genome or antigenome encodes a mutant RSV protein with an amino acid sequence that differs from the amino acid sequence set forth in SEQ ID NO: 6 only at position 107, wherein the residue at position 107 of the amino acid sequence is lysine, and wherein the NS1, NS2, N, P, M, and SH genes of the modified RSV genome or antigenome are each CPD.

In certain embodiments, the polynucleotide comprising a modified RSV genome or antigenome encodes a mutant RSV protein P with an amino acid sequence that differs from the amino acid sequence set forth in SEQ ID NO: 6 at at least position 229. In certain embodiments, the residue at position 229 of the amino acid sequence is glutamic acid. In a further embodiment, the polynucleotide comprising a modified RSV genome or antigenome encodes a mutant RSV protein with an amino acid sequence that differs from the amino acid sequence set forth in SEQ ID NO: 6 only at position 229, wherein the residue at position 229 of the amino acid sequence is glutamic acid, and preferably wherein the modified RSV genome or antigenome comprises at least one gene that is CPD. In a further embodiment, the polynucleotide comprising a modified RSV genome or antigenome encodes a mutant RSV protein with an amino acid sequence that differs from the amino acid sequence set forth in SEQ ID NO: 6 only at position 229, wherein the residue at position 229 of the amino acid sequence is glutamic acid, and wherein the NS1, NS2, N, P, M, and SH genes of the modified RSV genome or antigenome are each CPD.

In certain embodiments, the polynucleotide comprising a modified RSV genome or antigenome encodes a mutant RSV protein P with an amino acid sequence that differs from the amino acid sequence set forth in SEQ ID NO: 6 at at least position 234. In certain embodiments, the residue at position 234 of the amino acid sequence is histidine. In a further embodiment, the polynucleotide comprising a modified RSV genome or antigenome encodes a mutant RSV protein with an amino acid sequence that differs from the amino acid sequence set forth in SEQ ID NO: 6 only at position 234, wherein the residue at position 234 of the amino acid sequence is histidine, and preferably wherein the modified RSV genome or antigenome comprises at least one gene that is CPD. In a further embodiment, the polynucleotide comprising a modified RSV genome or antigenome encodes a mutant RSV protein with an amino acid sequence that differs from the amino acid sequence set forth in SEQ ID NO: 6 only at position 234, wherein the residue at position 234 of the amino acid sequence is histidine, and wherein the NS1, NS2, N, P, M, and SH genes of the modified RSV genome or antigenome are each CPD.

In certain embodiments, the polynucleotide comprising a modified RSV genome or antigenome encodes a mutant RSV protein P with an amino acid sequence that differs from the amino acid sequence set forth in SEQ ID NO: 6 at at least position 235. In certain embodiments, the residue at position 235 of the amino acid sequence is glycine. In a further embodiment, the polynucleotide comprising a modified RSV genome or antigenome encodes a mutant RSV protein with an amino acid sequence that differs from the amino acid sequence set forth in SEQ ID NO: 6 only at position 235, wherein the residue at position 235 of the amino acid sequence is glycine, and preferably wherein the modified RSV genome or antigenome comprises at least one gene that is CPD. In a further embodiment, the polynucleotide comprising a modified RSV genome or antigenome encodes a mutant RSV protein with an amino acid sequence that differs from the amino acid sequence set forth in SEQ ID NO: 6 only at position 235, wherein the residue at position 235 of the amino acid sequence is glycine, and wherein the NS1, NS2, N, P, M, and SH genes of the modified RSV genome or antigenome are each CPD.

In another embodiment, the polynucleotide comprising a modified RSV genome or antigenome encodes a mutant RSV P protein with an amino acid sequence that differs from the amino acid sequence set forth in SEQ ID NO: 6 at either position 27 or 28, wherein the residue at position 27 of the amino acid is asparagine or the residue at position 28 of the amino acid sequence is valine, wherein the modified RSV genome or antigenome comprises one or more of the following features: (a) encoding a mutant L protein that differs from the amino acid sequence set forth in SEQ ID NO: 13 only at position 2084, wherein the residue at position 2084 is proline; (b) encoding a mutant M protein that differs from the amino acid sequence set forth in SEQ ID NO: 7 only at position 123, wherein the residue at position 2084 is methionine; (c) comprising a nucleotide sequence SEQ ID NO: 16 corresponding to an NS2 5′ untranslated region (UTR); (d) comprising a nucleotide sequence SEQ ID NO: 18 corresponding to P gene start and 5′ UTR regions, and (e) comprising nucleotide sequence SEQ ID NO: 19 corresponding to a P 3′ UTR; and wherein the NS1, NS2, N, P, M, and SH genes of the modified RSV genome or antigenome are each CPD.

As used herein, the term “wild-type” refers to any naturally occurring RSV strain, including those isolated from a natural source, such as a mammalian subject. Exemplary wild-type RSV strain subgroups include, but are not limited to, human RSV subgroups A and B, which can be further classified into genotypes, such as A1, A2, A3, A4, A5, A6, A6, and other designations such as GA1-7, SAA1, NA1-4, and ON1-2, as well as B1, B2, B3, B4, and other designations such as GB1-4, SAB1-4, URU1-2, and BA1-10. Exemplary specific strains include RSV A2, RSV Long, RSV 8-60 and RSV 18537. The amino acid position numbering used herein is based on the amino acid sequence of the wild-type RSV A2 strain (GenBank accession number M74568, which is incorporated by reference herein) and all nucleotide sequences described are in positive-sense. The amino acid sequences of the 11 RSV proteins NS1, NS2, N, P, M, SH, G, F, M2-1, M2-2, and L are represented by SEQ ID NOs: 3-13, respectively.

The term “wild-type” is further intended to encompass the recombinant version of RSV strain A2 that is called D46. The complete sequence of D46 is shown in U.S. Pat. No. 6,790,449 (GenBank accession number KT992094, which is incorporated by reference herein). In some instances and publications, the parent virus and sequence is called D53 rather than D46, a book-keeping difference that refers to the strain of bacteria used to propagate the antigenomic cDNA and has no other known significance or effect. For the purposes of this disclosure, D46 and D53 are interchangeable. The nucleotide sequence of D46 differs from the sequence of RSV A2 strain M74568 in 25 nucleotide positions, which includes a 1-nt insert at position 1099.

The terms “parent” and “parental” used in the context of a virus, protein, or polynucleotide denotes the virus, protein, or polynucleotide from which another virus is derived. In some embodiments, the derived virus is made by recombinant means, or by culturing the parent virus under conditions that give rise to a mutation, and thus a different virus. In some embodiments, the terms refer to viral genomes and protein encoding sequences from which new sequences, which may be more or less attenuated, are derived. In some embodiments, the parent (or parental) viruses and sequences are wild type or naturally occurring prototypes or isolates of variants for which it is desired to obtain a more highly attenuated virus. In certain other embodiments, the parent (or parental) viruses are mutants specifically created or selected in the laboratory on the basis of real or perceived desirable properties. Accordingly, in certain embodiments, the parent (or parental) viruses that are candidates for attenuation are mutants of wild type. In other embodiments, the parent (or parental) viruses are naturally occurring viruses that have deletions, insertions, amino acid substitutions and the like. In further embodiments, the parent (or parental) viruses are mutants which have codon substitutions.

Those skilled in the art will recognize that the polynucleotide comprising the genome or antigenome of certain RSV variants may have nucleotide insertions or deletions that alter the encoded amino acid sequence, which in some cases can alter the position of one or more amino acid residues. For example, if a protein of another RSV strain had, in comparison with strain A2, two additional amino acids in the upstream end of the protein, this would cause the amino acid numbering of downstream residues relative to strain A2 to increase by an increment of two. However, because these strains share a large degree of sequence identity, those skilled in the art would be able to determine the location of corresponding sequences by simply aligning the nucleotide or amino acid sequence of the A2 reference strain with that of the strain in question. Therefore, it should be understood that the amino acid and nucleotide positions described herein, though specifically enumerated in the context of this disclosure, can correspond to other positions when a sequence shift has occurred or due to sequence variation between virus strains. In the comparison of a protein, or protein segment, or ORF, or gene, or genome, or genome segment between two or more related viruses, a “corresponding” amino acid or nucleotide residue is one that is exactly or approximately equivalent in function in the different RSV species.

In some embodiments, the amino acid sequence encoded by the genome or antigenome of the RSV variant may contain additional differences from the amino acid sequence encoded by the genome or antigenome of a parental and/or wild-type RSV strain. For instance, in some embodiments, the amino acid sequence encoded by the genome or antigenome of the RSV variant may comprise one or more changes in the F protein, e.g., the “HEK” mutation, which comprises two amino acid substitutions in the F protein, namely K66E and Q101P (described in Connors et al., Virology, 208: 478-484 (1995); Whitehead et al., J. Virol., 72: 4467-4471 (1998)). The introduction of the HEK amino acid assignments into the strain A2 F sequence of this disclosure results in an F protein amino acid sequence that is identical to that of an early-passage (human embryonic kidney cell passage 7, HEK-7) of the original clinical isolate of strain A2 (Connors et al., Virology, 208: 478-484 (1995); Whitehead et al., J. Virol., 72: 4467-4471 (1998)). It results in an F protein that is much less fusogenic and is believed to represent the phenotype of the original A2 strain clinical isolate (Liang et al., J. Virol., 89: 9499-9510 (2015)). The HEK F protein also forms a more stable trimer (Liang et al., J. Virol., 89: 9499-9510 (2015)). This may provide a more authentic and immunogenic form of the RSV F protein, possibly enriched for the highly immunogenic pre-fusion conformation (McLellan et al., Science, 340(6136): 1113-1117 (2013); McLellan et al., Science, 342(6158): 592-598 (2013)). Thus, mutations can be introduced with effects additional to effects on the magnitude of virus replication.

In some embodiments the amino acid sequence encoded by the genome or antigenome of the RSV variant may comprise one or more changes in the L protein, e.g., the stabilized 1030 or the “1030s” mutation which comprises 1321K(AAA)/51313(TCA) (Luongo et al., J. Virol., 86: 10792-10804 (2012)).

In some embodiments the amino acid sequence encoded by the genome or antigenome of the RSV variant may comprise one or more changes in the N protein, for example, an amino substitution such as T24A.

Deletion of the SH, NS1, and NS2 genes individually and in combination has been shown to yield viruses that retain their ability to replicate in cell culture but are attenuated in vivo in the following order of increasing magnitude: SH<NS2<NS1 (Bukreyev et al., J. Virol., 71: 8973-8982 (1997); Whitehead et al., J. Virol., 73: 3438-3442 (1999); Teng et al., J. Virol., 74: 9317-9321 (2000)). Therefore, in some embodiments, the genome or antigenome of the RSV variant comprises deletion or other mutations of the SH, NS2, or NS1 genes, or parts of their ORFs, is combined with one or more mutations described herein. For example, in some embodiments, the amino acid sequence encoded by the genome or antigenome of the RSV variant may comprise one or more changes in the SH protein, including an ablation or elimination of the SH protein. In some embodiments, the genome or antigenome of the RSV variant comprises a deletion in the SH gene. In some embodiments, the genome or antigenome of the RSV variant comprises a 419 nucleotide deletion at position 4197-4615 (4198-4616 of), denoted herein as the “ΔSH” mutation. This deletion results in the deletion of M gene-end, M/SH intergenic region, and deletion of the SH ORF. In some embodiments, the genome or antigenome of the RSV variant comprises one or more changes in the NS1 or the NS2 protein, which may result in an ablation or elimination of the protein. In some embodiments, the mutation encodes an amino substitution such as K51R in the NS2 protein.

In some embodiments, the genome or antigenome of the RSV variant encodes the “cp” mutation. This mutation refers to a set of five amino acid substitutions in three proteins (N (V2671), F (E218A and T5231), and L (C319Y and H1690Y)) which confer an approximate 10-fold reduction in replication in seronegative chimpanzees, and a reduction in illness (Whitehead et al., J. Virol., 72: 4467-4471 (1998)). The cp mutation has been associated with a moderate attenuation phenotype (Whitehead et al., J. Virol., 72: 4467-4471 (1999)).

In some embodiments, the genome or antigenome of the RSV variant encodes one or more amino acid substitutions in the L protein, including N43I, F521L, Q831L, M1169V, and/or Y1321N. Each substitution independently confers a temperature sensitive phenotype (i.e., an attenuated phenotype) and can optionally be combined with other modifications to the nucleotide sequence of the RSV variant, such as a single nucleotide change in the gene-start transcription signal of the M2 gene (GGGGCAAATA [SEQ ID NO: 14] to GGGGCAAACA [SEQ ID NO: 15], mRNA-sense), or the deletion of codon 1313 and amino acid substitution I1314L within the L protein.

Shifts in gene order (i.e., positional modifications moving one or more genes to a more promoter-proximal or promoter-distal location in the recombinant viral genome) in the genome or antigenome of the RSV variant can result in RSV viruses with altered biological properties. For example, RSV strains lacking NS1, NS2, SH, or G individually, or NS1 and NS2 together, or SH and G together have been shown to be attenuated in vitro, in vivo, or both. In particular, the G and F genes may be shifted, singly and in tandem, to a more promoter-proximal position relative to their parental and/or wild-type gene order. These two proteins normally occupy positions 7 (G) and 8 (F) in the RSV gene order (NS1-NS2-N-P-M-SH-G-F-M2-1-M2-2-L). In some embodiments, the order of the nucleotide sequences encoding the G and the F proteins may be reversed relative to the naturally occurring order.

In addition to the herein described mutations, in some embodiments, the polynucleotides of the invention can incorporate heterologous, coding or non-coding nucleotide sequences from any RSV or RSV-like virus, e.g., human, bovine, ovine, murine (pneumonia virus of mice), or avian (turkey rhinotracheitis virus) pneumovirus, or from another enveloped virus, e. g., parainfluenza virus (NV). Exemplary heterologous sequences include RSV nucleotide sequences from one human RSV strain combined with nucleotide sequences from a different human RSV strain. Alternatively, the RSV may incorporate nucleotide sequences from two or more, parental, wild-type, and/or mutant human RSV subgroups, for example a combination of human RSV subgroup A and subgroup B sequences. In still further embodiments, one or more human RSV coding or non-coding polynucleotides are substituted with a counterpart sequence from a heterologous RSV or non-RSV virus.

In addition to the polynucleotides and resulting RSV variants described herein, the disclosed viruses may be modified further as would be appreciated by those skilled in the art. For instance, the genome or antigenome of the RSV variant may have the ORF for one or more proteins removed or otherwise mutated or a heterologous gene from a different organism may be added thereto so that the genome or antigenome of the CPD RSV expresses or incorporates that protein upon infecting a cell and replicating. Furthermore, those skilled in the art will recognize that other previously defined mutations known to have an effect on RSV may be combined with one or more of any of the mutations described herein to produce a CPD RSV with desirable attenuation or stability characteristics.

In other embodiments, yet further modifications can be incorporated into genome or antigenome of the RSV variants that affect the strains' characteristics in ways other than attenuation. For instance, the one or more ORFs encoding RSV proteins may be codon-optimized within the context of the requirements for the RSV variants as described herein. Major protective antigens F and G can result in increased antigen synthesis. The F and/or G protein gene may be shifted upstream (i.e., closer to the promoter) to increase expression. The amino acid sequences encoding F and/or G protein can be modified to represent currently-circulating strains, which can be particularly important in the case of the divergent G protein, or to represent early-passage clinical isolates. Deletions or substitutions may be introduced into the nucleotide sequence encoding the G protein to obtain improved immunogenicity or other desired properties. For example, the CX3C fractalkine motif in the G protein might be ablated to improve immunogenicity (Chirkova et al., J. Virol., 87: 13466-13479 (2013)).

In some embodiments, the genome or antigenome of the CPD RSV comprises the nucleotide sequence of an ORF which has not been codon pair deoptimized and which has been replaced with a nucleotide sequence from a clinical isolate. For instance, the nucleotide sequence of an ORF encoding the RSV G protein may be replaced with a nucleotide sequence from a clinical isolate, such as A/Maryland/001/11. In some embodiments, the nucleotide sequence encoding the RSV F protein may be replaced with a nucleotide sequence from a clinical isolate, such as A/Maryland/001/11.

In some embodiments, a native or naturally occurring nucleotide sequence encoding one or more proteins of the RSV variant is replaced with a codon optimized sequence designed for increased expression in a selected host, for instance in humans. In some embodiments, the nucleotide sequence encoding the RSV F protein is replaced with a codon optimized sequence. In some embodiments, the nucleotide sequence of the ORF encoding the RSV F protein is replaced with the codon optimized sequence from a clinical isolate such as A/Maryland/001/11. In some embodiments, the nucleotide sequence encoding the RSV G protein is replaced with the codon optimized nucleotide sequence from a clinical isolate, such as A/Maryland/001/11.

In some embodiments, the genome or antigenome of the RSV variants further comprise a deletion of one or more non-translated sequences. In an embodiment, a portion of the downstream end of the SH gene is deleted, resulting in a mutation referred to as the “6120 Mutation” herein. The 6120 Mutation includes deletion of 112 nucleotides of the downstream non-translated region of the SH gene and the introduction of five translationally-silent point mutations in the last three codons and the termination codon of the SH gene (Bukreyev et al., J. Virol., 75: 12128-12140 (2001)). Presence of the term “LID” or “6120” in a recombinant virus name indicates that the recombinant virus contains the 6120 mutation.

The 6120 Mutation stabilizes the antigenomic cDNA in bacteria so that it can be more easily manipulated and prepared. In wild-type RSV strains, this mutation has been found to confer a 5-fold increase in replication efficiency in vitro (Bukreyev et al., J. Virol., 75: 12128-12140 (2001)), whereas it was not believed to increase replication efficiency in vivo.

The 6120 Mutation has been associated with increased replication in seronegative infants and children. Accordingly, the 6120 mutation provided another means to shift the level of attenuation. Moreover, the deletion of sequence exemplified by the 6120 Mutation in the downstream non-translated region of the SH gene, may involve any comparable genome sequence that does not contain a critical cis-acting signal (Collins and Karron, Fields Virology, 6th Edition (2013), pages 1086-1123). Genome regions that are candidates for deletion include, but are not limited to, non-translated regions in other genes, in the intergenic regions, and in the trailer region.

In certain embodiments, one or more genes of the genome or antigenome of the CPD RSV are replaced with, e.g., a bovine or other RSV counterpart, or with a counterpart or foreign gene from another respiratory pathogen such as PIV. Substitutions, deletions, and other modifications of RSV genes or gene segments in this context can include part or all of one or more of the NS1, NS2, N, P, M, SH, and L genes, or the M2-1 ORFs, or non-immunogenic parts of the G and F genes. Also, human RSV cis-acting sequences, such as promoter or transcription signals, can be replaced with, for example, their bovine RSV counterpart. In other embodiments, RSV variants comprise human attenuating genes or cis-acting sequences inserted into a bovine RSV genome or antigenome background.

Accordingly, RSV variants encoded by the polypeptide of the invention which is intended for administration to humans can be a human RSV that has been modified to contain genes from, for example, a bovine RSV or a PIV, such as for the purpose of attenuation. For example, by inserting a gene or gene segment from PIV, a bivalent vaccine to both PIV and RSV is provided. Alternatively, a heterologous RSV species, subgroup or strain, or a distinct respiratory pathogen such as PIV, may be modified, e.g., to contain genes that encode epitopes or proteins which elicit protection against human RSV infection. For example, the human RSV glycoprotein genes can be substituted for the bovine glycoprotein genes such that the resulting bovine RSV, which now bears the human RSV surface glycoproteins and would retain a restricted ability to replicate in a human host due to the remaining bovine genetic background, elicits a protective immune response in humans against human RSV strains.

In certain embodiments, a selected gene segment, such as one encoding a selected protein or protein region (for instance, a cytoplasmic tail, transmembrane domain or ectodomain, an epitope, a binding site or region, or an active site or region containing an active site) from one RSV strain, can be substituted for a counterpart gene segment from the same or different RSV strain or other source, to yield novel recombinants having desired phenotypic changes compared to parental and/or wild-type RSV strains. Such resulting strains may, for example, express a chimeric protein having a cytoplasmic tail and/or transmembrane domain of one RSV fused to an ectodomain of another RSV. Other exemplary embodiments of this type express duplicate protein regions, such as duplicate immunogenic regions.

As used herein, “counterpart” genes, gene segments, proteins or protein regions, are typically from heterologous sources (for example, from different RSV genes, or representing the same (i.e., homologous or allelic) gene or gene segment in different RSV strains). Generally, counterparts selected in this context share gross structural features, for example each counterpart may encode a comparable structural “domain,” such as a cytoplasmic domain, transmembrane domain, ectodomain, binding site or region, or epitope. Counterpart domains and their encoding gene segments embrace an assemblage of species having a range of size and amino acid (or nucleotide) sequence variations, which range is defined by a common biological activity among the domain or gene segment variants.

For example, in an embodiment, two selected protein domains encoded by counterpart gene segments may share substantially the same qualitative activity, such as providing a membrane spanning function, a specific binding activity, or an immunological recognition site. More typically, a specific biological activity shared between counterparts, for example, between selected protein segments or proteins, will be substantially similar in quantitative terms, i.e., they will not vary in respective quantitative activity profiles by more than 30%, preferably by no more than 20%, more preferably by no more than 5-10%.

In some embodiments, the RSV variant produced from a cDNA-expressed genome or antigenome can be any of the RSV or RSV-like strains, such as, human, bovine, or murine, or of any pneumovirus or metapneumovirus, such as pneumonia virus of mice or avian metapneumovirus. To elicit a protective immune response, the RSV variant may be one which is endogenous to the subject being immunized, such as human RSV being used to immunize humans. The genome or antigenome of endogenous RSV can be modified, however, to express RSV genes or gene segments from a combination of different sources, such as a combination of genes or gene segments from different RSV species, subgroups, or strains, or from an RSV and another respiratory pathogen such as human parainfluenza virus (PIV) (see, for example, Hoffman et al., J. Virol., 71: 4272-4277 (1997); Durbin et al., Virology, 235(2): 323-32 (1997); U.S. Pat. No. 7,208,161; WO 1998/053078; and the following plasmids for producing infectious PIV clones: p3/7(131) (ATCC 97990); p3/7(131)2G(ATCC 97889); and p218(131) (ATCC 97991); each deposited Apr. 18, 1997 under the terms of the Budapest Treaty with the American Type Culture Collection (ATCC) of 10801 University Blvd., Manassas, Va. 20110-2209, U.S.A., and accorded the aforementioned accession numbers.

### RSV Variant Having an Attenuated Phenotype

The invention provides an RSV variant having an attenuated phenotype that is encoded by the inventive polynucleotide as described herein.

The inventive RSV variant may be virus particle or a subviral particle. It may be present in a cell culture supernatant, isolated from the culture, or partially or completely purified. The RSV variant may also be lyophilized, and can be combined with a variety of other components for storage or delivery to a host, as desired.

The RSV variant of the invention are useful in various compositions to generate a desired immune response against RSV in a host susceptible to RSV infection. RSV variants of the invention are capable of eliciting a protective immune response in an infected human host, yet are sufficiently attenuated so as to not cause unacceptable symptoms of severe respiratory disease in the immunized host. In some embodiments, a live attenuated RSV vaccine comprises the RSV variant of the invention.

To select candidate vaccine viruses from the host of RSV variants provided herein, the criteria of viability, efficient replication in vitro, attenuation in vivo, immunogenicity, and phenotypic stability are determined according to well-known methods. The most desirable RSV viruses, in regards to generation of RSV vaccines and pharmaceutical compositions, should maintain viability, replicate sufficiently in vitro well under permissive conditions to make vaccine manufacture possible, have a stable attenuation phenotype, be well-tolerated, exhibit replication in an immunized host (albeit at lower levels), and effectively elicit production of an immune response in a vaccine sufficient to confer protection against serious disease caused by subsequent infection from wild-type virus. Given that no RSV vaccine has yet been approved, it appears that the previously reported attenuated RSV vaccine candidates do not sufficiently meet all of these criteria. The RSV variants of the invention, on the other hand, meet these criteria by exhibiting strong immunogenicity in vivo, at or near levels elicited by wild-type RSV, while still exhibiting stable attenuated replication.

RSV variants as described herein can be tested in various well known and generally accepted in vitro and in vivo models to confirm adequate attenuation, resistance to phenotypic reversion, and immunogenicity for vaccine use. The RSV variants, which can be a multiply attenuated, biologically derived or recombinant RSV, is tested in in vitro assays for temperature sensitivity of virus replication or “ts phenotype” and for the small plaque phenotype. The RSV variants may be further tested in animal models of RSV infection. A variety of animal models (e.g., murine, hamster, cotton rat, and primate) have been described and are known to those skilled in the art.

In an embodiment, an RSV variant may be employed as a “vector” for protective antigens of other pathogens, particularly respiratory tract pathogens such as parainfluenza virus (NV). For example, a recombinant RSV having a T11661 mutation may be prepared which incorporates sequences that encode protective antigens from PIV to produce infectious, attenuated vaccine virus.

### Production of RSV Variants Having Attenuated Phenotype

The invention provides a method for producing the inventive polynucleotide or inventive RSV variants as described herein.

The inventive polynucleotide or inventive RSV variant can be prepared by any suitable production technique, many of which are known in the art. For example, the inventive polynucleotide can be inserted into a suitable vector, which is used to transform a suitable host cell, e.g., a host cell permissive of RSV infection, which is replicated in a suitable culture, and then expressed to produce the inventive RSV variant. As such, the invention includes a vector comprising the inventive polynucleotide or inventive RSV variant, as well as a host cell transfected or transformed with the inventive polynucleotide or inventive RSV variant, e.g., by use of the inventive vector.

The inventive RSV variant can be produced from one or more isolated polynucleotides, for instance, one or more cDNAs. In on embodiment, cDNA encoding a RSV variant genome or antigenome is constructed for intracellular expression. In another embodiment, cDNA encoding all or part of a RSV variant genome or antigenome is coexpressed in vitro coexpression with the necessary viral proteins to form RSV variant. “RSV antigenome” refers to an isolated positive-sense polynucleotide molecule which serves as the template for the synthesis of progeny RSV genome. A cDNA is preferably constructed which is a positive-sense version of the RSV genome, corresponding to the replicative intermediate RNA, or antigenome, so as to minimize the possibility of hybridizing with positive-sense transcripts of the complementing sequences that encode proteins necessary to generate a transcribing, replicating nucleocapsid, i.e., sequences that encode N, P, Land M2-1 protein.

In certain embodiments, the invention provides a method for producing one or more purified RSV protein(s) which involves infecting a host cell permissive of RSV infection with a RSV variant under conditions that allow for RSV propagation in the infected cell. After a period of replication in culture, the cells are lysed, and the RSV is isolated therefrom. In other embodiments, one or more desired RSV proteins are purified after isolation of the virus, yielding one or more RSV proteins for vaccine, diagnostic, and other uses.

To propagate an RSV variant virus for vaccine use and other purposes, a number of different cell lines which allow for RSV growth may be used. RSV grows in a variety of human and animal cells. Preferred cell lines for propagating attenuated RS virus for vaccine use include DBSFRhL-2, MRC-5, and Vero cells. Highest virus yields are usually achieved with epithelial cell lines such as Vero cells. Cells are typically inoculated with virus at a multiplicity of infection ranging from about 0.001 to 1.0, or more and are cultivated under conditions permissive for replication of the virus, e.g., at about 30-37° C. and for about 3-10 days, or as long as necessary for the virus to reach an adequate titer. Temperature-sensitive viruses often are grown using 32° C. as the “permissive temperature.” Virus is removed from cell culture and separated from cellular components, typically by well-known clarification procedures, such as centrifugation, and may be further purified as desired using procedures well known to those skilled in the art.

The herein described method for producing attenuated recombinant RSV mutants can be used to yield infectious viral or subviral particles, or derivatives thereof. An infectious virus is comparable to the wild-type RSV virus particle and is infectious “as is.” An infectious virus can directly infect fresh cells. An infectious subviral particle typically is a subcomponent of the virus particle which can initiate an infection under appropriate conditions. For example, a nucleocapsid containing the genomic or antigenomic RNA and the N, P, L and M2-1 proteins is an example of a subviral particle which can initiate an infection if introduced into the cytoplasm of cells. Subviral particles provided by an embodiment of the invention include viral particles which lack one or more protein(s), protein segment(s), or other viral component(s) not essential for infectivity.

Other embodiments provide a cell or a cell-free lysate containing an expression vector which comprises the inventive polynucleotide, and an expression vector (the same or different vector) comprising one or more isolated polynucleotide molecules encoding the N, P, L, and M2-2 proteins of RSV. In further embodiments, one or more of these proteins is expressed from genome or antigenome cDNA. Upon expression, the genome or antigenome and N, P, L, and M2-2 proteins combine to produce an infectious RSV viral or sub-viral particle.

### Pharmaceutical Composition

The invention provides a pharmaceutical composition comprising the inventive RSV variant and at least one excipient. The inventive pharmaceutical composition desirably comprises an immunologically effective amount of the inventive RSV variant. In some embodiments, a live attenuated RSV vaccine comprises the inventive pharmaceutical composition.

The inventive pharmaceutical composition can be prepared in any suitable manner, many of which are known in the art. The excipient can be any suitable excipient, such as a carrier. Suitable carriers include, for example, buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vaccine vehicles and additives, including those that are particularly useful in formulating modified live vaccines, are known or will be apparent to those skilled in the art. See, e.g., Remington's Pharmaceutical Science, 18th ed., Mack Publishing (1990), which is incorporated herein by reference.

The inventive pharmaceutical composition may comprise one or more additional immunomodulatory components such as, for instance, an adjuvant or cytokine, among others. Adjuvants that can be used in the compositions include, but are not limited to, the RIBI adjuvant system (Ribi Inc., Hamilton, Mont.), alum, mineral gels such as aluminum hydroxide gel, oil-in-water emulsions, water-in-oil emulsions such as, for example, Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN™ adjuvant, saponin, Quil A or other saponin fraction, monophosphoryl lipid A, ionic polysaccharides, and Avridine lipid-amine adjuvant. Non-limiting examples of oil-in-water emulsions useful in the vaccine of the invention include modified SEAM62 and SEAM 1/2 formulations. Modified SEAM62 is an oil-in-water emulsion containing 5% (v/v) squalene (Sigma), 1% (v/v) SPAN™ 85 detergent (ICI Surfactants), 0.7% (v/v) TWEEN™ 80 detergent (ICI Surfactants), 2.5% (v/v) ethanol, 200 μg/ml Quil A, 100 μg/ml cholesterol, and 0.5% (v/v) lecithin. Modified SEAM 1/2 is an oil-in-water emulsion comprising 5% (v/v) squalene, 1% (v/v) SPAN™ 85 detergent, 0.7% (v/v) Tween 80 detergent, 2.5% (v/v) ethanol, 100 μg/ml Quil A, and 50 μg/ml cholesterol. Other immunomodulatory agents that can be included in the composition include, e.g., one or more interleukins, interferons, or other known cytokines. Additional adjuvant systems permit for the combination of both T-helper and B-cell epitopes, resulting in one or more types of covalent T-B epitope linked structures, which may be additionally lipidated, such as those described in WO 2006/084319, WO 2004/014957, and WO 2004/014956.

The inventive pharmaceutical composition contains as an active ingredient an immunogenically effective amount of a RSV variant as described herein. Biologically derived or recombinant RSV strains can be administered directly to a host as a vaccine used directly in a vaccine formulation or composition. The biologically derived or recombinantly modified virus may be introduced into a host with a physiologically acceptable carrier and/or adjuvant. Useful carriers are well known in the art and include, for example, water, buffered water, 0.4% saline, 0.3% glycine, hyaluronic acid, and the like. The resulting aqueous solutions can be packaged for use “as is” provided in frozen form that is thawed prior to use, or lyophilized, with the lyophilized preparation being combined with a sterile solution prior to administration. The compositions may contain pharmaceutically acceptable auxiliary substances as required to approximate physiological conditions, which include, but are not limited to, pH adjusting and buffering agents, tonicity adjusting agents, wetting agents and the like, for example, sodium acetate, sodium lactate, sodium chloride, potassium chloride, calcium chloride, sucrose, magnesium sulfate, phosphate buffers, HEPES (4-(2-hydroxyethyl)-1-piperazineethanesulfonic acid) buffer, sorbitan monolaurate, and triethanolamine oleate. Acceptable adjuvants include incomplete Freund's adjuvant, aluminum phosphate, aluminum hydroxide, or alum, which are materials well known in the art. Preferred adjuvants also include Stimulon™ QS-21 (Aquila Biopharmaceuticals, Inc., Worchester, Mass.), MPL™ (3-O-deacylated monophosphoryl lipid A; RIM ImmunoChem Research, Inc., Hamilton, Mont.), and interleukin-12 (Genetics Institute, Cambridge, Mass.).

### Multivalent RSV Vaccine Composition

The invention provides a multivalent RSV vaccine composition comprising a first RSV variant of the invention, a second RSV variant of the invention, and, optionally, one or more additional RSV variants of the invention, wherein the first, second, and optional additional RSV variants have different nucleotide sequences.

The inventive multivalent RSV vaccine composition can comprise one or more excipients or other components as described herein for the inventive pharmaceutical composition.

In some embodiments, the vaccine or pharmaceutical composition comprises an RSV variant that elicits an immune response against a single RSV strain or antigenic subgroup, e.g., A or B, or against multiple RSV strains or subgroups. In this regard, an RSV variant can be combined in vaccine formulations with other RSV vaccine strains or subgroups having different immunogenic characteristics for more effective protection against one or multiple RSV strains or subgroups. They may be administered in a vaccine mixture, or administered separately in a coordinated treatment protocol. to elicit more effective protection against one RSV strain, or against multiple RSV strains or subgroups.

### Vaccination Method

The invention provides a method of vaccinating an animal. The vaccination method comprises administering the inventive RSV variant, preferably in the form of the inventive pharmaceutical composition, to an animal. Further provided is a method of inducing an immune response comprising administering the vaccine or pharmaceutical composition.

In certain embodiments, a live attenuated RSV variant vaccine (or RSV variant pharmaceutical composition) is administered, wherein the vaccine or pharmaceutical composition comprises the RSV variant encoded by a polynucleotide of the invention as described herein.

The vaccine and pharmaceutical compositions may be administered by any suitable method, including but not limited to, via injection, nasal spray, nasal droplets, topical application, aerosol delivery, or oral inoculation. In some embodiments, the compositions may be administered intranasally or subcutaneously or intramuscularly. In some embodiments, the compositions may be administered to the upper respiratory tract. The compositions can be administered to an individual seronegative for antibodies to RSV or possessing transplacentally acquired maternal antibodies to RSV.

The animal to which the vaccine or pharmaceutical composition is administered can be any mammal susceptible to infection by RSV or a closely related virus and capable of generating a protective immune response to antigens of the vaccine strain. Thus, suitable animals include humans, non-human primates, bovine, equine, swine, ovine, caprine, lagamorph, rodents, such as mice or cotton rats, etc. Accordingly, the invention provides methods for creating vaccines for a variety of human and veterinary uses.

In the case of humans, the RSV variant can be administered according to well established human RSV vaccine protocols (Karron et al., JID, 191: 1093-104 (2005)). Briefly, adults or children are inoculated intranasally via droplet with an immunogenically effective dose of RSV vaccine, typically in a volume of 0.5 ml of a physiologically acceptable diluent or carrier. This has the advantage of simplicity and safety compared to parenteral immunization with a non-replicating vaccine. It also provides direct stimulation of local respiratory tract immunity, which plays a major role in resistance to RSV. Further, this mode of vaccination effectively bypasses the immunosuppressive effects of RSV specific maternally-derived serum antibodies, which typically are found in the very young. Also, while the parenteral administration of RSV antigens can sometimes be associated with immunopathologic complications, this has never been observed with a live virus.

For humans, the precise amount of RSV variant vaccine administered and the timing and repetition of administration will be determined by various factors, including the patient's state of health and weight, the mode of administration, and the nature of the formulation. Dosages will generally range from about 3.0 log 10 to about 6.0 log 10 plaque forming units (“PFU”) or more of virus per patient, more commonly from about 4.0 log 10 to 5.0 log 10 PFU virus per patient. In one embodiment, about 5.0 log 10 to 6.0 log 10 PFU per patient may be administered during infancy, such as between 1 and 6 months of age, and one or more additional booster doses could be given 2-6 months or more later. In another embodiment, young infants could be given a dose of about 5.0 log 10 to 6.0 log 10 PFU per patient at approximately 2, 4, and 6 months of age, which is the recommended time of administration of a number of other childhood vaccines. In still another embodiment, an additional booster dose could be administered at approximately 10-15 months of age. The vaccine formulations and pharmaceutical compositions should provide a quantity of RSV variant of the invention sufficient to effectively stimulate or induce an anti-RSV immune response (an “immunogenically effective amount”).

In some embodiments, neonates and infants are given multiple doses of RSV vaccine to elicit sufficient levels of immunity. Administration may begin within the first month of life, and at intervals throughout childhood, such as at two months, four months, six months, one year and two years, as necessary to maintain sufficient levels of protection against natural RSV infection. In other embodiments, adults who are particularly susceptible to repeated or serious RSV infection, such as, for example, health care workers, day care workers, family members of young children, the elderly, individuals with compromised cardiopulmonary function, are given multiple doses of RSV vaccine to establish and/or maintain protective immune responses. Levels of induced immunity can be monitored by measuring amounts of neutralizing secretory and serum antibodies, and dosages adjusted or vaccinations repeated as necessary to maintain desired levels of protection. Further, different vaccine viruses may be indicated for administration to different recipient groups. For example, an engineered RSV strain expressing a cytokine or an additional protein rich in T cell epitopes may be particularly advantageous for adults rather than for infants. Vaccines produced in accordance with the present invention can be combined with viruses of the other subgroup or strains of RSV to achieve protection against multiple RSV subgroups or strains, or selected gene segments encoding, for example, protective epitopes of these strains can be engineered into one RSV variant clone as described herein. In such embodiments, the different viruses can be in admixture and administered simultaneously or present in separate preparations and administered separately. For example, as the F glycoproteins of the two RSV subgroups differ by only about 11% in amino acid sequence, this similarity is the basis for a cross-protective immune response as observed in animals immunized with RSV or F antigen and challenged with a heterologous strain. Thus, immunization with one strain may protect against different strains of the same or different subgroup.

Upon immunization with a RSV vaccine composition, the host responds to the vaccine by producing antibodies specific for RSV virus proteins, for example, F and G glycoproteins. In addition, innate and cell-mediated immune responses are induced, which can provide antiviral effectors as well as regulating the immune response. As a result of the vaccination the host becomes at least partially or completely immune to RSV infection, or resistant to developing moderate or severe RSV disease, particularly of the lower respiratory tract.

The resulting immune response can be characterized by a variety of methods. These include taking samples of nasal washes or sera for analysis of RSV-specific antibodies, which can be detected by tests including, but not limited to, complement fixation, plaque neutralization, enzyme-linked immunosorbent assay, luciferase-immunoprecipitation assay, and flow cytometry. In addition, immune responses can be detected by assay of cytokines in nasal washes or sera, ELISPOT of immune cells from either source, quantitative RT-PCR or microarray analysis of nasal wash or serum samples, and restimulation of immune cells from nasal washes or serum by re-exposure to viral antigen in vitro and analysis for the production or display of cytokines, surface markers, or other immune correlates measured by flow cytometry or for cytotoxic activity against indicator target cells displaying RSV antigens. In this regard, individuals are also monitored for signs and symptoms of upper respiratory illness.

### Method of Inducing an Immune Response

The invention provides a method of inducing an immune response in an animal. The method comprises administering the inventive RSV variant to an animal. The inventive RSV variant can be administered in the same forms and/or same ways as described herein for the inventive vaccination method.

Provided herein is a method for stimulating the immune system of an individual to elicit an immune response against RSV in a mammalian subject. The method comprises administering an immunogenic formulation of an immunologically sufficient or effective amount of a RSV variant in a physiologically acceptable carrier and/or adjuvant.

The RSV variant of the invention is useful in various compositions to generate a desired immune response against RSV in a host susceptible to RSV infection. Attenuated variant RSV strains of the invention are capable of eliciting a protective immune response in an infected human host, yet are sufficiently attenuated so as to not cause unacceptable symptoms of severe respiratory disease in the immunized host. The attenuated virus or subviral particle may be present in a cell culture supernatant, isolated from the culture, or partially or completely purified. The virus may also be lyophilized, and can be combined with a variety of other components for storage or delivery to a host, as desired.

### Method of Producing RSV Vaccine

The invention provides a method of producing an RSV vaccine. The method comprises expressing the polynucleotide of the invention as described herein in a cell. The aspects of the method, e.g., the nature of the cell, are the same as described herein for the production of the inventive RSV variant.

As used herein, the terms “recipient,” “individual,” “subject,” “host,” and “patient” are used interchangeably and refer to any mammalian subject for whom vaccination is desired (e.g., humans). “Mammal” and “animal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

### Examples of Non-Limiting Aspects of the Disclosure

Aspects, including embodiments, of the invention described herein may be beneficial alone or in combination, with one or more other aspects or embodiments. Without limiting the foregoing description, certain non-limiting aspects of the disclosure numbered (1)-(84) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A polynucleotide encoding a recombinant respiratory syncytial virus (RSV) variant having an attenuated phenotype comprising a modified RSV genome or antigenome that encodes a mutant RSV protein P that differs from a parental RSV protein P at one or more amino acid residues.

(2) The polynucleotide of aspect 1, wherein the mutant RSV protein P has an amino acid sequence that differs from the amino acid sequence set forth in SEQ ID NO: 6 at one or more positions selected from the group consisting of 19-34, 107, 229, 234, and 235.

(3) The polynucleotide of any of aspects 1-2, wherein the mutant RSV protein P has an amino acid sequence that differs from the amino acid sequence set forth in SEQ ID NO: 6 at at least position 25.

(4) The polynucleotide of aspect 3, wherein the residue at position 25 of the amino acid sequence is threonine or asparagine.

(5) The polynucleotide of any of aspects 1-4, wherein the mutant RSV protein P has an amino acid sequence that differs from the amino acid sequence set forth in SEQ ID NO: 6 at at least position 27.

(6) The polynucleotide of aspect 5, wherein the residue at position 27 of the amino acid sequence is glutamic acid or asparagine.

(7) The polynucleotide of any of aspects 1-6, wherein the mutant RSV protein P has an amino acid sequence that differs from the amino acid sequence set forth in SEQ ID NO: 6 at at least position 28.

(8) The polynucleotide of aspect 7, wherein the residue at position 28 of the amino acid sequence is valine, isoleucine, proline, leucine, or serine.

(9) The polynucleotide of any of aspects 1-8, wherein the mutant RSV protein P has an amino acid sequence that differs from the amino acid sequence set forth in SEQ ID NO: 6 at at least position 32.

(10) The polynucleotide of aspect 9, wherein the residue at position 32 of the amino acid sequence is threonine.

(11) The polynucleotide of any of aspects 1-10, wherein the mutant RSV protein P has an amino acid sequence that differs from the amino acid sequence set forth in SEQ ID NO: 6 at at least position 34.

(12) The polynucleotide of aspect 11, wherein the residue at position 34 of the amino acid sequence is serine.

(13) The isolated polynucleotide of any of aspects 1-12, wherein one or more ORFs of the modified RSV genome or antigenome is codon-pair deoptimized.

(14) The polynucleotide of any of aspects 1-13, wherein the nucleotide sequence of the modified RSV genome or antigenome encoding one or more of RSV proteins NS1, NS2, N, P, M, and SH has about 70% to about 95% identity with the nucleotide sequence of a parental RSV genome or antigenome encoding the same one or more of RSV proteins NS1, NS2, N, P, M, and SH

(15) The polynucleotide of aspect 14, wherein the nucleotide sequence of the modified RSV genome or antigenome encoding RSV protein NS1 has about 75% to about 95% identity with the nucleotide sequence of the parental RSV genome or antigenome encoding RSV protein NS1.

(16) The polynucleotide of aspect 15, wherein the nucleotide sequence of the modified RSV genome or antigenome encoding RSV NS1 protein has about 87% identity with the nucleotide sequence of the parental RSV genome or antigenome encoding RSV protein NS1.

(17) The polynucleotide of aspect 16, wherein the nucleotide sequence of the modified RSV genome or antigenome encoding RSV NS1 protein is represented by nucleotides 99 to 518 of SEQ ID NO: 2.

(18) The polynucleotide of any of aspects 14-17, wherein the nucleotide sequence of the modified RSV genome or antigenome encoding RSV protein NS2 has about 75% to about 95% identity with the nucleotide sequence of the parental RSV genome or antigenome encoding RSV protein NS2.

(19) The polynucleotide of aspect 18, wherein the nucleotide sequence of the modified RSV genome or antigenome encoding RSV NS2 protein has about 88% identity with the nucleotide sequence of the parental RSV genome or antigenome encoding RSV protein NS2.

(20) The polynucleotide of aspect 19, wherein the nucleotide sequence of the modified RSV genome or antigenome encoding RSV NS2 protein is represented by nucleotides 628 to 1002 of SEQ ID NO: 2.

(21) The polynucleotide of any of aspects 14-20, wherein the nucleotide sequence of the modified RSV genome or antigenome encoding RSV protein N has about 70% to about 90% identity with the nucleotide sequence of the parental RSV genome or antigenome encoding RSV protein N.

(22) The polynucleotide of aspect 21, wherein the nucleotide sequence of the modified RSV genome or antigenome encoding RSV N protein has about 80% identity with the nucleotide sequence of the parental RSV genome or antigenome encoding RSV protein N.

(23) The polynucleotide of aspect 22, wherein the nucleotide sequence of the modified RSV genome or antigenome encoding RSV N protein is represented by nucleotides 1141 to 2316 of SEQ ID NO: 2.

(24) The polynucleotide of any of aspects 14-23, wherein the nucleotide sequence of the modified RSV genome or antigenome encoding RSV protein P has about 75% to about 95% identity with the nucleotide sequence of the parental RSV genome or antigenome encoding RSV protein P.

(25) The polynucleotide of aspect 24, wherein the nucleotide sequence of the modified RSV genome or antigenome encoding RSV P protein has about 84% identity with the nucleotide sequence of the parental RSV genome or antigenome encoding RSV protein P.

(26) The polynucleotide of aspect 25, wherein the nucleotide sequence of the modified RSV genome or antigenome encoding RSV P protein is represented by nucleotides 2347 to 3072 of SEQ ID NO: 2.

(27) The polynucleotide of any of aspects 14-26, wherein the nucleotide sequence of the modified RSV genome or antigenome encoding RSV protein M has about 75% to about 95% identity with the nucleotide sequence of the parental RSV genome or antigenome encoding RSV protein M.

(28) The polynucleotide of aspect 27, wherein the nucleotide sequence of the modified RSV genome or antigenome encoding RSV M protein has about 83% identity with the nucleotide sequence of the parental RSV genome or antigenome encoding RSV protein M.

(29) The polynucleotide of aspect 28, wherein the nucleotide sequence of the modified RSV genome or antigenome encoding RSV M protein is represented by nucleotides 3262 to 4032 of SEQ ID NO: 2.

(30) The polynucleotide of any of aspects 14-29, wherein the nucleotide sequence of the modified RSV genome or antigenome encoding RSV protein SH has about 85% to about 95% identity with the nucleotide sequence of the parental RSV genome or antigenome encoding RSV protein SH.

(31) The polynucleotide of aspect 30, wherein the nucleotide sequence of the modified RSV genome or antigenome encoding RSV SH protein has about 92% identity with the nucleotide sequence of the parental RSV genome or antigenome encoding RSV protein SH.

(32) The polynucleotide of aspect 31, wherein the nucleotide sequence of the modified RSV genome or antigenome encoding RSV SH protein is represented by nucleotides 4304 to 4498 of SEQ ID NO: 2.

(33) The polynucleotide of any of aspects 14-32, where an amino acid sequence of the one or more of RSV proteins NS1, NS2, N, P, M and SH encoded by the nucleotide sequence of the modified RSV genome or antigenome is at least 99% identical to an amino acid sequence of the same one or more of RSV proteins NS1, NS2, N, P, M and SH encoded by the nucleotide sequence of the parental RSV genome or antigenome.

(34) A recombinant RSV variant comprising the isolated polynucleotide of any of aspects 1-33.

(35) A pharmaceutical composition comprising the recombinant RSV variant of aspect 34 and at least one excipient.

(36) A multivalent RSV vaccine composition comprising a recombinant RSV variant of any of aspects 1-35, a second recombinant RSV variant of any of aspects 1-35, and, optionally, one or more additional recombinant RSV variants of any of aspects 1-35, wherein the first, second, and optional additional recombinant RSV variants have different nucleotide sequences.

(37) A method of vaccinating an animal, comprising administering the pharmaceutical composition of aspect 35 or the multivalent RSV vaccine composition of aspect 36 to an animal.

(38) A method of inducing an immune response in an animal, comprising administering the recombinant RSV variant of aspects 34 or 35 to an animal.

(39) The method of aspect 38, wherein the recombinant RSV variant is administered via injection, nasal spray, nasal droplets, topical application, aerosol delivery, or oral inoculation.

(40) The method of any one of aspects 37-39, wherein the animal is a mammal.

(41) The method of any one of aspects 37-39, wherein the animal is a human.

(42) A method of producing a recombinant RSV variant vaccine, comprising expressing the polynucleotide of any of aspects 1-33 in a cell.

(43) A polynucleotide encoding a recombinant respiratory syncytial virus (RSV) variant having an attenuated phenotype comprising a modified RSV genome or antigenome that encodes a mutant RSV protein P that differs from a wild-type RSV protein P at one or more amino acid residues.

(44) The polynucleotide of aspect 43 wherein the mutant RSV protein P has an amino acid sequence that differs from the amino acid sequence of wild-type RSV protein P set forth in SEQ ID NO: 6 at one or more positions selected from the group consisting of 19-34, 107, 229, 234, and 235.

(45) The polynucleotide of any of aspects 43-44, wherein the mutant RSV protein P has an amino acid sequence that differs from the amino acid sequence of the wild-type RSV protein P set forth in SEQ ID NO: 6 at at least position 25.

(46) The polynucleotide of aspect 45, wherein the residue at position 25 of the amino acid sequence of the mutant RSV protein P is threonine or asparagine.

(47) The polynucleotide of any of aspects 1-46, wherein the mutant RSV protein P has an amino acid sequence that differs from the amino acid sequence of the wild-type RSV protein P set forth in SEQ ID NO: 6 at at least position 27.

(48) The polynucleotide of aspect 47, wherein the residue at position 27 of the amino acid sequence of the mutant RSV protein P is glutamic acid or asparagine.

(49) The polynucleotide of any of aspects 1-48, wherein the mutant RSV protein P has an amino acid sequence that differs from the amino acid sequence of the wild-type RSV protein P set forth in SEQ ID NO: 6 at at least position 28.

(50) The polynucleotide of aspect 49, wherein the residue at position 28 of the amino acid sequence of the mutant RSV protein P is valine, isoleucine, proline, leucine, or serine.

(51) The polynucleotide of any of aspects 1-50, wherein the mutant RSV protein P has an amino acid sequence that differs from the amino acid sequence of the wild-type RSV protein P set forth in SEQ ID NO: 6 at at least position 32.

(52) The polynucleotide of aspect 51, wherein the residue at position 32 of the amino acid sequence of the mutant RSV protein P is threonine.

(53) The polynucleotide of any of aspects 1-52, wherein the mutant RSV protein P has an amino acid sequence that differs from the amino acid sequence of the wild-type RSV protein P set forth in SEQ ID NO: 6 at at least position 34.

(54) The polynucleotide of aspect 53, wherein the residue at position 34 of the amino acid sequence of the mutant RSV protein P is serine.

(55) The isolated polynucleotide of any of aspects 1-54, wherein one or more ORFs of the modified RSV genome or antigenome is codon-pair deoptimized.

(56) The polynucleotide of any of aspects 1-55, wherein a nucleotide sequence of the modified RSV genome or antigenome encoding one or more of RSV proteins NS1, NS2, N, P, M, and SH has about 70% to about 95% identity with a nucleotide sequence of a wild-type RSV genome or antigenome encoding the same one or more of RSV proteins NS1, NS2, N, P, M, and SH.

(57) The polynucleotide of aspect 56, wherein the nucleotide sequence of the modified RSV genome or antigenome encoding RSV protein NS1 has about 75% to about 95% identity with the nucleotide sequence of the wild-type RSV genome or antigenome encoding RSV protein NS1.

(58) The polynucleotide of aspect 57, wherein the nucleotide sequence of the modified RSV genome or antigenome encoding RSV NS1 protein has about 87% identity with the nucleotide sequence of the wild-type RSV genome or antigenome encoding RSV protein NS1.

(59) The polynucleotide of aspect 58, wherein the nucleotide sequence of the modified RSV genome or antigenome encoding RSV NS1 protein is represented by nucleotides 99 to 518 of SEQ ID NO: 2.

(60) The polynucleotide of any of aspects 1-59, wherein the nucleotide sequence of the modified RSV genome or antigenome encoding RSV protein NS2 has about 75% to about 95% identity with the nucleotide sequence of the wild-type RSV genome or antigenome encoding RSV protein NS2.

(61) The polynucleotide of aspect 60, wherein the nucleotide sequence of the modified RSV genome or antigenome encoding RSV NS2 protein has about 88% identity with the nucleotide sequence of the wild-type RSV genome or antigenome encoding RSV protein NS2.

(62) The polynucleotide of aspect 61, wherein the nucleotide sequence of the modified RSV genome or antigenome encoding RSV NS2 protein is represented by nucleotides 628 to 1002 of SEQ ID NO: 2.

(63) The polynucleotide of any of aspects 56-62, wherein the nucleotide sequence of the modified RSV genome or antigenome encoding RSV protein N has about 70% to about 90% identity with the nucleotide sequence of the wild-type RSV genome or antigenome encoding RSV protein N.

(64) The polynucleotide of aspect 63, wherein the nucleotide sequence of the modified RSV genome or antigenome encoding RSV N protein has about 80% identity with the nucleotide sequence of the wild-type RSV genome or antigenome encoding RSV protein N.

(65) The polynucleotide of aspect 64, wherein the nucleotide sequence of the modified RSV genome or antigenome encoding RSV N protein is represented by nucleotides 1141 to 2316 of SEQ ID NO: 2.

(66) The polynucleotide of any of aspects 56-65, wherein the nucleotide sequence of the modified RSV genome or antigenome encoding RSV protein P has about 75% to about 95% identity with the nucleotide sequence of the wild-type RSV genome or antigenome encoding RSV protein P.

(67) The polynucleotide of aspect 66, wherein the nucleotide sequence of the modified RSV genome or antigenome encoding RSV P protein has about 84% identity with the nucleotide sequence of the wild-type RSV genome or antigenome encoding RSV protein P.

(68) The polynucleotide of aspect 67, wherein the nucleotide sequence of the modified RSV genome or antigenome encoding RSV P protein is represented by nucleotides 2347 to 3072 of SEQ ID NO: 2.

(69) The polynucleotide of any of aspects 56-68, wherein the nucleotide sequence of the modified RSV genome or antigenome encoding RSV protein M has about 75% to about 95% identity with the nucleotide sequence of the wild-type RSV genome or antigenome encoding RSV protein M.

(70) The polynucleotide of aspect 69, wherein the nucleotide sequence of the modified RSV genome or antigenome encoding RSV M protein has about 83% identity with the nucleotide sequence of the wild-type RSV genome or antigenome encoding RSV protein M.

(71) The polynucleotide of aspect 70, wherein the nucleotide sequence of the modified RSV genome or antigenome encoding RSV M protein is represented by nucleotides 3262 to 4032 of SEQ ID NO: 2.

(72) The polynucleotide of any of aspects 56-71, wherein the nucleotide sequence of the modified RSV genome or antigenome encoding RSV protein SH has about 85% to about 95% identity with the nucleotide sequence of the wild-type RSV genome or antigenome encoding RSV protein SH.

(73) The polynucleotide of aspect 72, wherein the nucleotide sequence of the modified RSV genome or antigenome encoding RSV SH protein has about 92% identity with the nucleotide sequence of the wild-type RSV genome or antigenome encoding RSV protein SH.

(74) The polynucleotide of aspect 73, wherein the nucleotide sequence of the modified RSV genome or antigenome encoding RSV SH protein is represented by nucleotides 4304 to 4498 of SEQ ID NO: 2.

(75) The polynucleotide of any of aspects 56-74, where an amino acid sequence of the one or more of RSV proteins NS1, NS2, N, P, M and SH encoded by the nucleotide sequence of the modified RSV genome or antigenome is at least 99% identical to an amino acid sequence of the same one or more of RSV proteins NS1, NS2, N, P, M and SH encoded by the nucleotide sequence of the wild-type RSV genome or antigenome.

(76) A recombinant RSV variant comprising the isolated polynucleotide of any of aspects 1-75.

(77) A pharmaceutical composition comprising the recombinant RSV variant of aspect 76 and at least one excipient.

(78) A multivalent RSV vaccine composition comprising a recombinant RSV variant of any of aspects 1-77, a second recombinant RSV variant of any of aspects 1-77, and, optionally, one or more additional recombinant RSV variants of any of aspects 1-77, wherein the first, second, and optional additional recombinant RSV variants have different nucleotide sequences.

(79) A method of vaccinating an animal, comprising administering the pharmaceutical composition of aspect 77 or the multivalent RSV vaccine composition of aspect 78 to an animal.

(80) A method of inducing an immune response in an animal, comprising administering the recombinant RSV variant of aspects 76 or 77 to an animal.

(81) The method of aspect 80, wherein the recombinant RSV variant is administered via injection, nasal spray, nasal droplets, topical application, aerosol delivery, or oral inoculation.

(82) The method of any one of aspects 79-81, wherein the animal is a mammal.

(83) The method of any one of aspects 79-81, wherein the animal is a human.

(84) A method of producing a recombinant RSV variant vaccine, comprising expressing the polynucleotide of any of aspects 1-75 in a cell.

### EXAMPLES

### Example 1

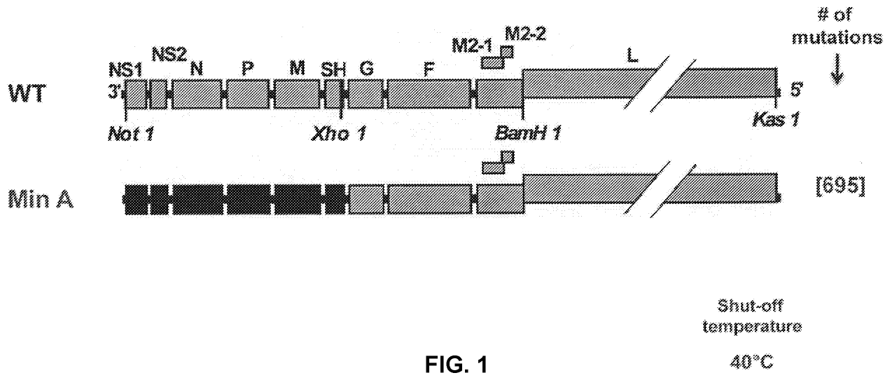

Viruses: All the viruses were derived from the RSV backbone named D46/6120, which is a version of wild-type (wt) RSV strain A2 (GenBank accession number KT992094). This backbone contains a 112 nucleotide deletion in the downstream non-translated region of the SH gene and 5 silent nucleotide point mutations involving the last three codons and termination codon of the SH ORF, which stabilize the RSV cDNA during propagation in E. coli without affecting virus replication in vitro and in mouse (Bukreyev et al., “Granulocyte-macrophage colony-stimulating factor expressed by recombinant respiratory syncytial virus attenuates viral replication and increases the level of pulmonary antigen-presenting cells,” J. Virol., 75(24): 12128-12140 (2001)). The design and rescue of Min A has been previously described (Le Nouen et al., “Attenuation of human respiratory syncytial virus by genome-scale codon-pair deoptimization,” Proc. Natl. Acad. Sci. USA., 111(36): 13169-13174 (2014)). Briefly, a previously described computational algorithm (Coleman et al., “Virus attenuation by genome-scale changes in codon pair bias,” Science, 320(5884): 1784-1787 (2008)) was used to generate RSV NS1, NS2, N, P, M and SH ORFs sequences that contained an increased number of codon pairs that are underrepresented in the human ORFs (i.e codon-pair deoptimization, CPD). The CPD process resulted in the introduction of 65, 60, 241, 143, 163 and 23 silent mutations in the NS1, NS2, N, P, M, and SH ORFs, respectively, for a total of 695 silent mutations in Min A (Le Nouen et al. (2014), supra). The amino acid sequence of Min A is identical to the wild type RSV strain A2. Min A was found to be temperature sensitive, with a shut-off temperature of 40° C. (FIG. 1). As used in this example, sequence numbering of the RSV genomes is based on recombinant RSV strain A2 (GenBank Accession number KT992094) from which nt 4499-4610 inclusive (i.e., the deletion in RSV 6120) have been deleted.

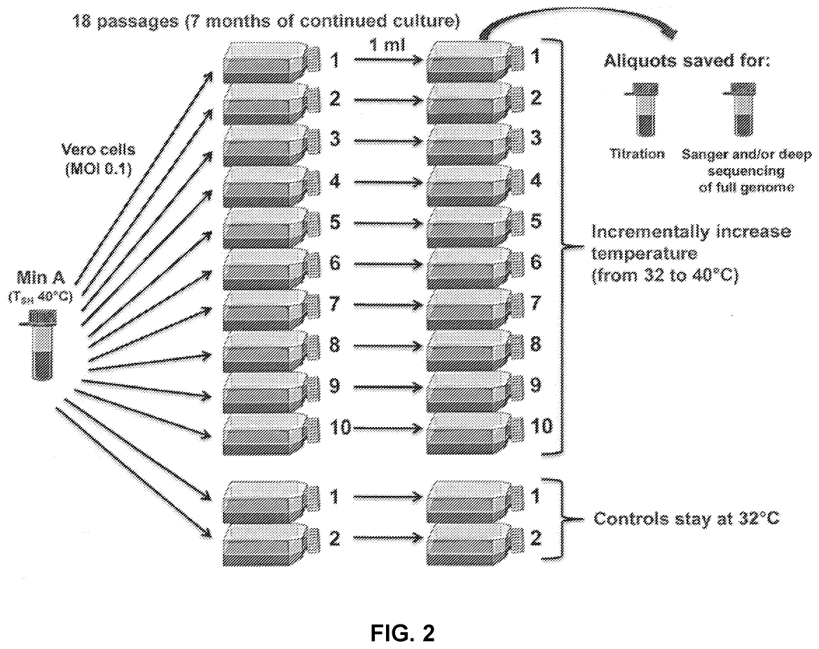

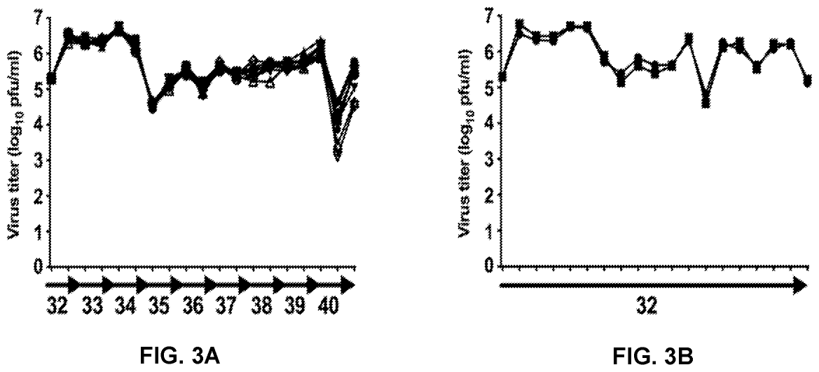

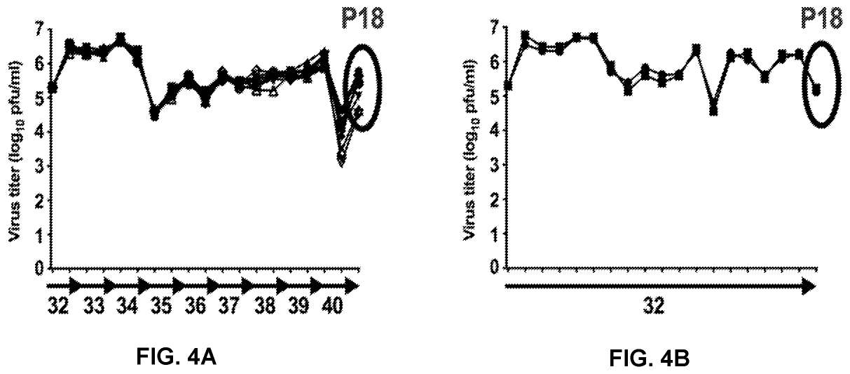

Identification of mutations in Min A strains after exposure to step-wise temperature stress: Twelve 25 cm²-flasks of Vero cells were inoculated with Min A at an initial multiplicity of infection (MOI) of 0.1 plaque forming units (pfu) per cell. Each flask generated its own individual lineage. Ten of the 12 flasks were subjected to a temperature stress by increasing the temperature by 1° C. every other passage starting from 32° C. to 40° C. for a total of 18 passages. Two additional flasks were passaged in parallel 18 times at the permissive temperature of 32° C. as controls. When extensive syncytia formation was observed or when the cells started to detach (typically between day 6 and 11), virus from each flask was harvested by scraping infected cells in media, followed by vortex of 30 seconds, and clarification of supernatant by centrifuge. Aliquots were then snap frozen in dry ice and stored in −80° C. Then, 20% of the harvested virus (1 ml of the 5 ml total) were used to inoculate the following passage (FIG. 2). At the end of each passage, aliquots of virus were snap frozen in dry ice for titration and sequencing by Sanger sequencing and/or deep sequencing as indicated (FIGS. 3A-B and 4A-B).

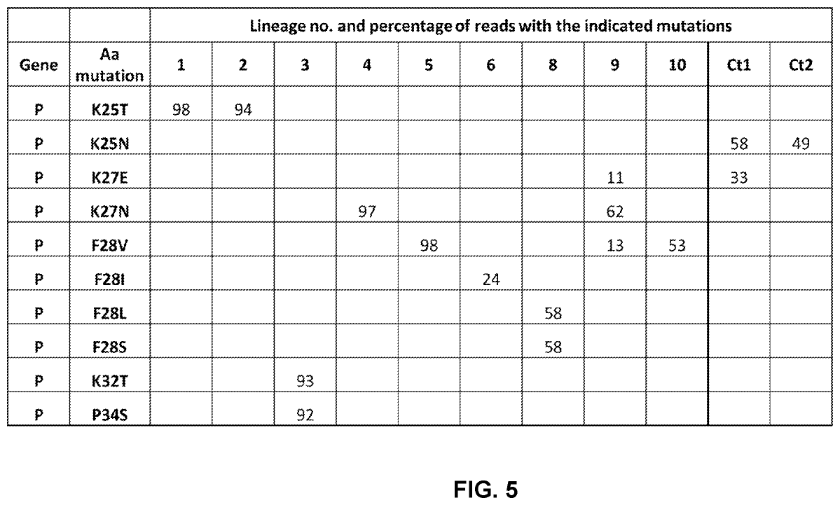

Mutations that accumulated over the passages were identified using Ion Torrent whole genome deep sequencing: Viral RNA was extracted from clarified supernatants collected at the end of the last passage (P18) using the QiaAmp Viral RNA extraction kit (Qiagen) and reverse transcribed using superscript II reverse transcriptase (RT, Thermofisher) following the manufacturer recommendations. The cDNA was amplified by PCR using RSV specific primers and a high-fidelity DNA polymerase (pfx DNA polymerase, Thermofisher) as described previously (Le Nouen et al., “Genetic stability of genome-scale deoptimized RNA virus vaccine candidates under selective pressure,” Proc. Natl. Acad. Sci. U.S.A., 114(3): E386-E95 (2017)), and PCR amplicons were purified using the QIAquick PCR purification kit (Qiagen). Then, Ion torrent deep sequencing was performed as previously described (Le Nouen et al. (2017), supra). The only sequences that were not directly determined for each genome were the positions of the outer-most primers, namely nucleotides 1-23 and 15,062-15,111. DNA sequences were compared using VariantCaller 3.2 software (Ion Torrent). Parameters of the analysis pipeline were set at the Ion Torrent default somatic variant configuration. A nucleotide variant was called if the variant occurred >50 times with an average read depth of 1000× and a P-value <10-7 (Quality score >70) as previously described (Le Nouen et al. (2017), supra). The raw read data were also manually verified using the IVG genome browser (The Broad Institute). Identified mutations detected at a frequency of more than 5 percent are set forth in Table 1 (see also FIG. 5).

TABLE 1
Mutations detected at a frequency of ≥5% in each of the 9 lineages of Min
A at the end of the temperature stress test as well as in each of the 2 controls.

| | | | Lineage no. and percentage of reads with the indicated | | | | | | | | | | |
|---|---|---|---|---|---|---|---|---|---|---|---|---|---|
| | nt | aa | mutation* | | | | | | | | | | |
| Gene | mutation | mutation | 1 | 2 | 3 | 4 | 5 | 6 | 7 | 8 | 9 | Ct1 | Ct2 |
| NS1 | t440g^{†‡} | S114R | | | | | | | 6 | | | | |
| NS1-NS2 | t612c | / | | | | | 91 | | | | | | |
| intergenic | | | | | | | | | | | | | |
| NS2 | a723g | R32R | | | | 9 | | | | | | | |
| | | (silent) | | | | | | | | | | | |
| NS2-N | a1138g | / | | | | 59 | | | | | | | |
| intergenic | | | | | | | | | | | | | |
| N | c1301a^{†} | T54N | | | | | | | | 49 | | | |
| N | t1380c^{†‡} | I80I | | | | | | 7 | | | | | |
| | | (silent) | | | | | | | | | | | |
| N | t1410c^{†‡} | V90V | | | | | | 6 | | | | | |
| | | (silent) | | | | | | | | | | | |
| N | a1459c^{†} | K107Q | | 57 | | | | | | | | | |
| N | a1460g^{†} | K107R | 7 | | | | | | | | | | |
| N | a1547g^{†} | K136R | | | 42 | | | | | | | | |
| N | a1749g^{†‡§} | K203K | | | | 94 | | | | | | | |
| | | (silent) | | | | | | | | | | | |
| N | c1929t^{†‡} | L263L | | | | | | 18 | | | | | |
| | | (silent) | | | | | | | | | | | |
| N | t2151c^{†‡§} | Y337Y | | | | | | | | 62 | | | |
| | | (silent) | | | | | | | | | | | |
| P gene start | c2334t | / | | | | | 68 | | | | | | |
| P | t2364c | P6P | | | | | | | 64 | | 61 | | |
| | | (silent) | | | | | | | | | | | |
| P | a2376t | G10G | | | | 77 | | | | | | | |
| | | (silent) | | | | | | | | | | | |
| P | t2388c^{†‡§} | N14N | | | | | | | | | 45 | | |
| | | (silent) | | | | | | | | | | | |
| P | a2402t | K19I | | | | | | 44 | | | | | |
| P | t2405a | F20Y | | | | | | | | | 42 | | |
| P | a2419g | K25E | | | | | | | | | | | 36 |
| P | a2420c | K25T | 98 | 94 | | | | | | | | | |
| P | g2421t | K25N | | | | | | | | | | 58 | 49 |
| P | t2424c^{†‡§} | G26G | | | | | | | | 10 | | | |
| | | (silent) | | | | | | | | | | | |
| P | a2425g^{†} | K27E | | | | | | | | 11 | | 33 | |
| P | g2427t^{†‡} | K27N | | | | 97 | | | | 62 | | | |
| P | t2428g^{†} | F28V | | | | | 98 | | | 13 | 53 | | |
| P | t2428a^{†} | F28I | | | | | | 24 | | | | | |
| P | t2428c^{†} | F28L | | | | | | | 58 | | | | |
| P | t2429c^{†} | F28S | | | | | | | 58 | | | | |
| P | t2428c & | F28P | | | | | | | 58 | | | | |
| | t2429c^{†} | | | | | | | | | | | | |
| P | a2441c | K32T | | | 93 | | | | | | | | |
| P | c2446t^{†} | P34S | | | 92 | | | | | | | | |
| P | t2577c^{†‡§} | P77P | | 27 | | | | | 73 | | | | |
| | | (silent) | | | | | | | | | | | |
| P | g2665a | E107K | | | | | | | | 13 | | | |
| P | g3032a^{†} | G229E | | | 91 | | | | | | | | |
| P | a3046c^{†} | N234H | 78 | | | | | | | | | | |
| P | a3050g | D235G | | | | 8 | | | | | | | |
| P-M | a3195g | / | | | | | 89 | | | | | | |
| intergenic | | | | | | | | | | | | | |
| M | a3629t^{†} | K123M | | | | | 62 | | | | | | |
| M | g3770a | R170K | | | | | | | | | 15 | | |
| M-SH | t4211a | / | | | | | 9 | | | | | | |
| intergenic | | | | | | | | | | | | | |
| SH | t4351c | P16P | | | | | | | | | | 12 | |
| | | (silent) | | | | | | | | | | | |
| SH | t4352c | Y17H | | | | | | | | | | 12 | |
| SH | t4411c^{†‡§} | I36I | | | | | | | | | | 12 | |
| | | (silent) | | | | | | | | | | | |
| SH | t4426c^{†‡} | L41L | | | | | | | | | | 12 | |
| | | (silent) | | | | | | | | | | | |
| SH | t4449c | V49A | | | | | | | | | | 11 | |
| SH gene | t4619c | / | | | | | | | | | | 12 | |
| end | | | | | | | | | | | | | |
| SH gene | t4620c | / | | | | | | | | | | 13 | |
| end | | | | | | | | | | | | | |
| SH-G | a4662g | / | | | | | | | | 53 | | | |
| intergenic | | | | | | | | | | | | | |
| G | c5141t | R151R | | | | | | | | | | 15 | |
| | | (silent) | | | | | | | | | | | |
| G | a5167g | N160S | | | | | | | | | | | 17 |
| G | a5169g | N161D | | | | | | | | | | | 18 |
| G | a5170g | N161S | | | | | | | | | | | 30 |
| G | a5194g | N169S | | | | | | | | | | | 21 |
| G | a5424g | T246A | | | | | | | | | | | 23 |
| G | a5429g | L247L | | | | | | | | | | | 25 |
| | | (silent) | | | | | | | | | | | |
| G | a5477g | E263E | | | | | | | | | | | 13 |
| | | (silent) | | | | | | | | | | | |
| G | a5478g | T264A | | | | | | | | | | | 14 |
| F | a6996t | K445N | | | | 9 | | | | | | | |
| F | a7163g | Q501R | | | | | | | | | 14 | | |
| F | a7194g | E511E | | | | | | | | 6 | | | |
| | | (silent) | | | | | | | | | | | |
| M2-1 | a7616g | R4G | | | | | 38 | | | | | | |
| M2-1 | a7647g | H14R | | | | | | | | 8 | | | |
| M2-1 | t7674c | F23S | | | | | 8 | | | | | | |
| M2-1 | a7754g | M50V | | | | | | | | | 78 | | |
| M2-1 | a8091g | K162R | | | | | | 54 | | | | | |
| M2-1 | a8134t | P176P | | | | 7 | | | | | | | |
| | | (silent) | | | | | | | | | | | |
| M2-2 | c8227t | T23I | | | | | 7 | | | | | | 20 |
| M2-2 | t8343c | S62P | | | | | | | | | | | |
| M2-2 | t8356g | I66S | | | | 5 | | | | | | | |
| L | t8950c | V151A | | | | 59 | | | | | | | |
| L | t8951c | V151V | | | | 59 | | | | | | | |
| | | (silent) | | | | | | | | | | | |
| L | t9453c | C319R | | 9 | | | | 56 | | 11 | | | |
| L | a10434t | M646L | | 6 | | | | | | | | | |
| L | t10622c | F708F | | | | | | | | 45 | | | |
| | | (silent) | | | | | | | | | | | |
| L | a10782g | N762D | 77 | | | | | | | | | | |
| L | a11361t | I955L | | | | | | | | 49 | | | |
| L | g11574a | D1026N | 99 | 97 | | | | | | | | | |
| L | a12435t | S1313C | | | | | | | 9 | | | | |
| L | g13194a | V1566I | | | | | | | 69 | | | | |
| L | a13208g | I1570M | | | | | | | | 58 | | | |
| L | g13679a | K1727K | | | | | | | | 11 | | | |
| | | (silent) | | | | | | | | | | | |
| L | t13739c | S1747S | | | | | | 18 | | | | | |
| | | (silent) | | | | | | | | | | | |
| L | t13753c | I1752T | | | | | | 19 | | | | | |
| L | t13754c | I1752I | | | | | | 19 | | | | | |
| | | (silent) | | | | | | | | | | | |
| L | t13797c | L1767L | | | | | | 12 | | | | | |
| | | (silent) | | | | | | | | | | | |
| L | a13898g | I1800M | | | | | | | 69 | | | | |
| L | t14105c | P1869P | | | | | | | 32 | | | | |
| | | (silent) | | | | | | | | | | | |
| L | t14276c | F1926F | | | | | | | | | | 12 | |
| | | (silent) | | | | | | | | | | | |
| L | t14669c | F2057F | | | | | | 68 | | | | | |
| | | (silent) | | | | | | | | | | | |
| L | t14748c | S2084P | | | 62 | | 93 | | | | | | |
| 5′ end | t15163c | / | | | | 7 | | | | | | | |
| trailer | | | | | | | | | | | | | |

Nucleotide numbering is based on RSV sequence M74568.
“/” indicates that the amino acid mutation is not applicable for this particular mutation as the given mutation is localized in a nontranslated region.
*Percentage of reads with the indicated mutation; only mutations present in ≥5% of the reads are shown.
^{†}Mutations involving a codon that had been changed as part of CPD of NS1, NS2, N, P, M, or SH
^{‡}Mutations involving a nucleotide that had been changed as part of CPD of NS1, NS2, N, P, M, or SH.
^{§}Mutation involving a nucleotide that had been changed as part of CPD of NS1, NS2, N, P, M, or SH and that restored WT sequence.

Introduction of mutations into Min A cDNA backbones: Certain mutations in the RSV P protein identified during the passages in vitro were reintroduced into Min A cDNA backbone using the QuikChange Lightning Site-Directed Mutagenesis Kit (Agilent Technologies) following manufacturer's instructions. Each newly generated Min A derived cDNA was completely sequenced by Sanger sequencing using a set of specific primers.

Rescue of recombinant RSVs by reverse genetics: Five μg of cDNAs encoding for Min A, Min A-derived viruses or wild-type RSV full-length genome, 2 μg of N and P encoding cDNAs and 1 μg of M2 and L encoding cDNAs were co-transfected using Lipofectamine 2000 (Thermofisher) in a P6-well of BSR-T7 cells cultured in GMEM media supplemented with 3% FBS and 2% GMEM Amino Acids. After overnight incubation at 37° C., transfected BSR-T7 cells were gently scraped into the media and transferred to a 50% confluent 75 cm2 flask of Vero cells cultured in OptiMEM supplemented with 5% FBS and 1% L-glutamine. Viruses were harvested between 11 and 14 days when extensive syncytia formation was observed by scraping infected cells in media, followed by vortex of 30 seconds, and clarification of supernatant by centrifuge. Aliquots of the passage 1 (P1) virus stock were snap frozen in dry ice and stored in −80° C. Virus titer was determined by plaque assay at 32° C. Then, the P1 virus stock was amplified once on Vero cells to generate a P2 virus working stock. To do so, T225 cm2 flasks of Vero cells were infected at MOI of 0.05 or 0.01 pfu per cell with the P1 virus stock. Virus was harvested and titered as described above. The complete sequence of each virus stock was confirmed by Sanger and/or Ion Torrent deep sequencing of overlapping reverse transcribed PCR amplicons.

Testing of Min A Variants with P Protein Mutations:

Certain P mutations identified between aa 25 and 34 were associated with a loss of the temperature sensitivity of Min A. The genetic basis for the loss of the is phenotype of Min A during the stress test experiment in FIG. 1 was investigated. Six prominent P missense mutations were chosen that were present in two lineages at a level of ≥45% of reads, or were present in a single lineage at a level of ≥90% of reads (Table 1). The six P missense mutations were: K25T, K25N, K27N, F28V, K32T and P34S. These were re-introduced individually by site-directed mutagenesis into the Min A antigenomic cDNA and rescued by reverse genetics, and the complete genome sequences were confirmed by Sanger sequencing.

The temperature sensitivity of the Min A-derived viruses were compared to Min A and wt RSV (Table 2). In this study, the titer of Min A at 40° C. was 2.3 log₁₀ lower than at 32° C., whereas the titer of wt RSV at 40° C. was 0.4 log₁₀ lower than at 32° C. Thus, the difference in the reduction in titer of Min A compared to wt RSV at the same temperatures was 1.9 log₁₀, which was slightly less than the difference of ≥2.0 log₁₀ that formally defines the temperature-sensitive phenotype (see the footnote to Table 2). While this was somewhat less than the 2.6 log₁₀ difference that was previously observed (Le Nouen et al., PNAS USA, 111(36): 13169-74 (2014)), it was sufficient to evaluate possible effects of the mutations. We found that most of the P missense mutations that we had introduced into Min A substantially increased its ability to form plaques at 40° C. In particular, Min A containing the mutation P[K25T], P[K27N], or P[K32T] had titers at 40° C. that were only 0.3-0.4 log₁₀ lower than at 32° C., similar to wt RSV. In contrast, mutation P[P34S] was the least effective in compensating for the temperature sensitivity of Min A, resulting in a titer that was 1.7 log₁₀ lower at 40° C. than at 32° C.

TABLE 2
Temperature sensitivity of the CPD RSVs on Vero cells.

| | Virus titer (log₁₀ PFU per ml) at | | | | | | | |
|---|---|---|---|---|---|---|---|---|
| | indicated temperature (° C.)^{a} | | | | | | | |
| Virus | 32 | 35 | 36 | 37 | 38 | 39 | 40 | T_{SH} |
| Min A | 6.2 | 6.5 | 6.4 | 6.1 | 6.1 | 5.1 | 3.9 | >40 |
| Min A-P[K25T] | 7.0 | 7.0 | 7.0 | 7.0 | 6.9 | 6.8 | 6.6 | >40 |
| Min A-P[K25N] | 7.0 | 7.0 | 6.9 | 6.9 | 6.8 | 6.7 | 5.5 | >40 |
| Min A-P[K27N] | 6.8 | 6.8 | 6.8 | 6.8 | 6.8 | 6.6 | 6.5 | >40 |
| Min A-P[F28V] | 6.7 | 6.8 | 6.8 | 6.7 | 6.8 | 6.6 | 6.0 | >40 |
| Min A-P[K32T] | 6.9 | 7.0 | 6.9 | 6.9 | 6.8 | 6.9 | 6.8 | >40 |
| Min A-P[P34S] | 5.7 | 5.7 | 5.5 | 5.2 | 6.0 | 4.7 | 4.0 | >40 |
| wt RSV | 6.9 | 6.9 | 6.9 | 6.9 | 6.8 | 6.8 | 6.5 | >40 |

^{a}The ts phenotype for each virus was evaluated by assessing virus growth on Vero at the indicated temperatures utilizing temperature controlled water-baths. TSH is defined as the lowest restrictive temperature at which there is a reduction in plaque number compared to 32° C. that is 100-fold or greater than that observed for wt RSV at the two temperatures. The ts phenotype is defined as having a TSH of 40° C. or less.

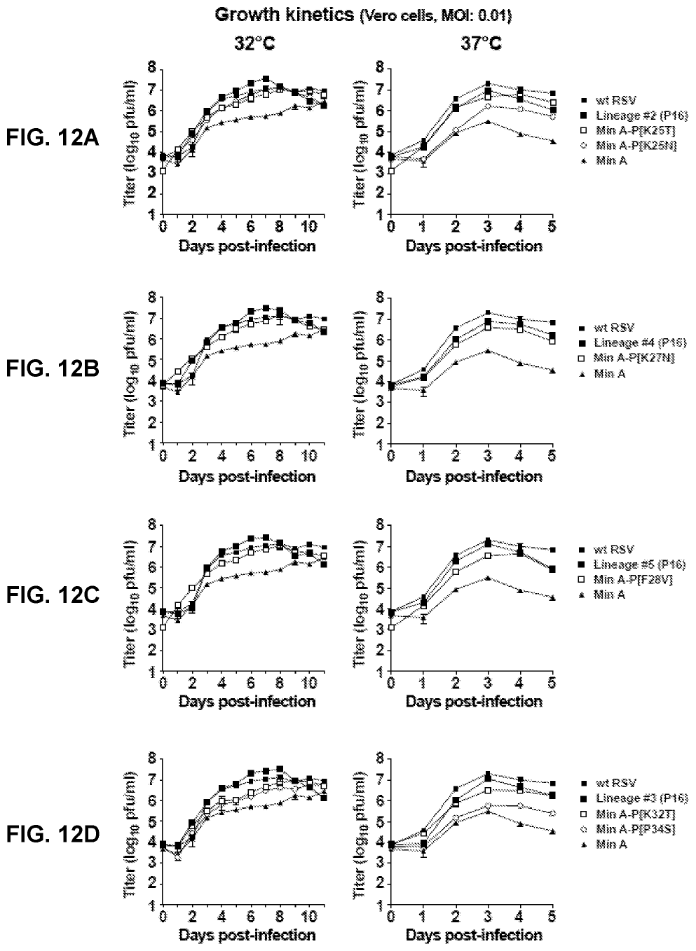

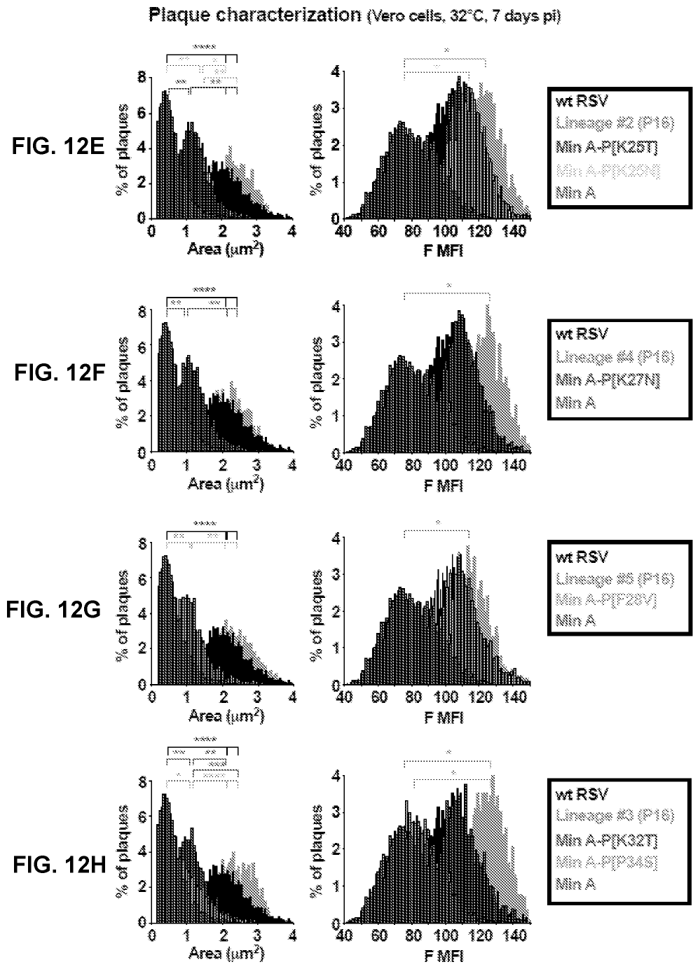

The missense P mutations increased Min A fitness in vitro. The effects of the single P mutations on multicycle replication of Min A were investigated (FIGS. 12A-H). Vero cells were infected side-by-side in duplicate at an MOI of 0.01 PFU/cell with: Min A; the indicated Min A-derived viruses bearing single P mutations; P16 of lineages #2, #3, #4, or #5; or wt RSV. P16 lineages were included in this set of experiments because of the limited material from the P18 lineages and the high virus titers obtained at P16. The presence in P16 of the prominent P mutations that we had originally identified in the P18 lineages was confirmed by Sanger sequencing. Cells were incubated at the permissive temperature of 32° C. (FIG. 2B, left column) or the physiological temperature of 37° C. (right column).

At 32° C., wt RSV replication peaked at 107.1 PFU/ml on day 7 pi, as typically observed (FIGS. 12A-D, left column). As expected, Min A replication was reduced by about 10-fold compared to wt RSV (105.9 PFU/ml at day 7) and reached a maximal titer only at day 11 (106.5 PFU/ml). All of the Min A-derived mutants replicated more efficiently than Min A at 32° C.; the P mutations at aa positions 25, 27, 28 and 32 conferred increases in Min A replication of up to 10-fold to peak titers of 106.8-107.2 PFU/ml, comparable to wt virus.

Comparable results were obtained at 37° C., except that maximum virus titers were reached at day 3-4 pi instead of day 7-8 pi that was observed at 32° C. The peak titer for wt RSV was 107.3 PFU/ml, as typically observed. Min A replication at 37° C. was approximately 60-fold reduced compared to wt RSV (peak titers of 105.5 PFU/ml), consistent with previous results (Le Nouen et al., PNAS USA, 111(36): 13169-74 (2014)) and further confirming its is phenotype. The presence of each of the P mutations at aa position 25, 27, 28 and 32 increased replication of Min A by about 5- to 15-fold to peak titers of 106.2-106.7 PFU/ml. The mutation P[P34S], which had the least effect on temperature sensitivity and replication at 32° C., still increased Min A replication at 37° C. by two-fold. Replication of P16 of lineages #2, #4, #5 and #3 (also was increased compared to Min A, and reached 106.9-107.1 PFU/ml. Thus, the P mutations conferred increased replication at 32° C. and 37° C. in comparison to Min A.

We also evaluated the effect of the P mutations on Min A fitness by characterizing the plaque sizes and the level of RSV F expression of individual plaques on Vero cells at 32° C. (FIGS. 12E-H). Min A virus and wt RSV were included as controls, as well as P16 supernatants from lineages #2, #4, #5 and #3 (FIGS. 12E-H). Plaque sizes of all Min A-derived P mutants were significantly increased compared to Min A but remained intermediate between Min A and wt RSV. In contrast, the plaque sizes of the P16 virus stocks equaled or slightly exceeded that of wt RSV. With regard to the magnitude of expression of RSV F, all of the P mutations except for [P34S] increased the expression of RSV F per plaque, although not to the level of the P16 stocks or wt RSV. The increase compared to Min A was statistically significant for mutation P[K25N]. These data further confirmed that the individual P mutations improved Min A fitness.

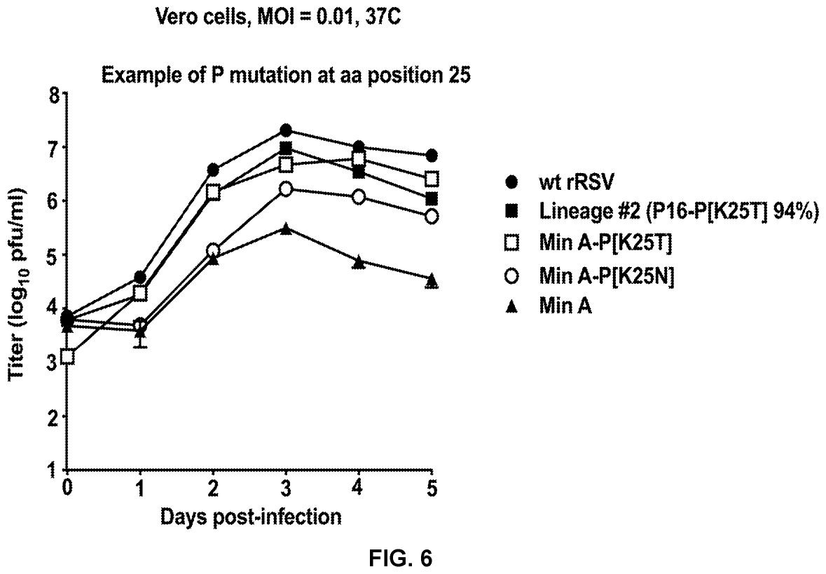

Multicycle growth kinetic. Multi-cycle growth kinetics were performed as previously described in Le Nouen et al. (2017), supra (see FIG. 6). Briefly, duplicate confluent monolayers of Vero cells in 6-well plates were infected in duplicate with the indicated viruses at an MOI of 0.01 pfu/cell and incubated at 32° C. or 37° C. Viruses were collected daily by scraping infected cells into media, followed by vortexing for 30 sec, and clarification of the supernatant by centrifugation. Virus inoculum and clarified supernatants were snap frozen and stored at −80° C., and virus titers were determined later by immunoplaque assay as described above.

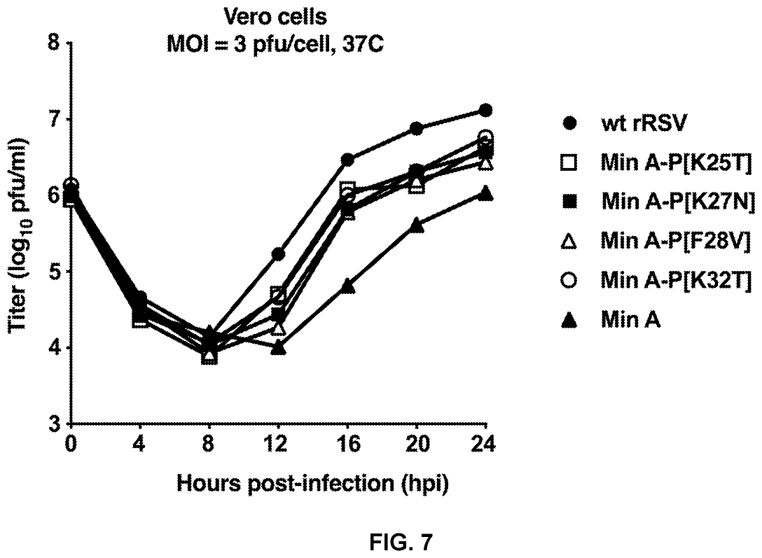

Single-cycle growth kinetic. Single cycle replication experiments were performed as previously described in Le Nouen et al. (2017), supra (see FIG. 7). Briefly, Vero cells in 6-well plates were infected at an MOI of 3 pfu/well at 37° C. with the indicated viruses. Every four hours from four to 24 h post-infection, one well per virus was used to harvest virus and determined the virus titers by plaque assay.

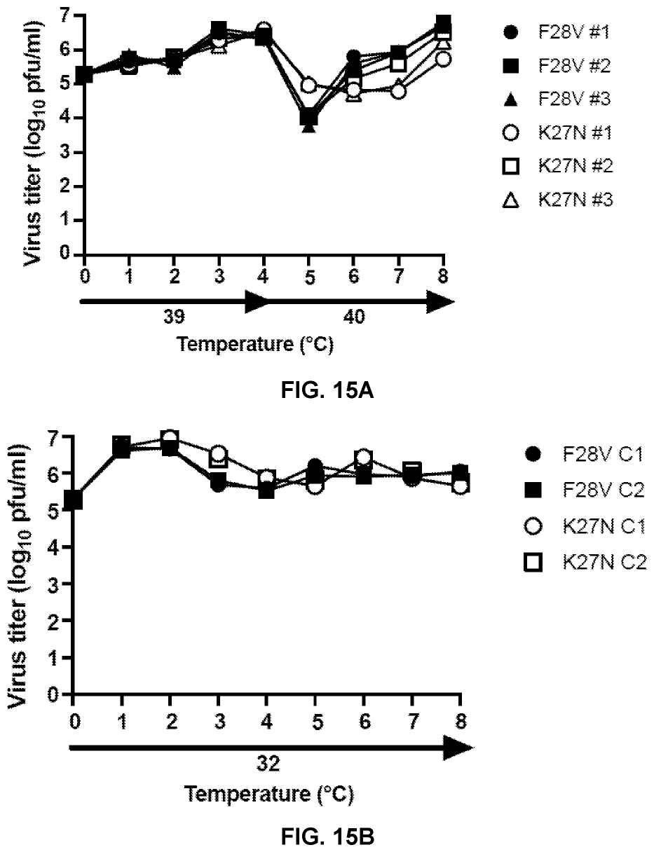

Genetic stability: The genetic stability of the Min A-P[K27N] and Min A-P[F28V] viruses was evaluated in a temperature stress test involving 4 passages at 39° C. and 4 passages at 40° C., corresponding to 2 months of continuous culture (FIG. 15A). Each virus was also passaged in parallel at the permissive temperature of 32° C. as control (FIG. 15B). Sanger sequencing of the complete genomes of the 3 different stressed replicates and the 2 control replicates was performed at the end of the final passage (P8).

In the case of Min A-P[K27N, no prominent (≥45% of reads) mutations were found at P8 in the three stressed replicates nor in the two control replicates. Three subdominant (≥5% to <44% of reads) missense mutations in L ([N1473D], [N1475D] and [I114771R]) were found in one stressed lineage. Thus, Min A-P[K27N] exhibited substantially increased genetic stability compared to Min A.

In the case of Min A-P[F28V], no prominent mutations were found in two of three stressed replicates nor in one control replicate. In the remaining stressed lineage, four subdominant missense mutations were detected in M2-1 ([H14R], [N40S], [M50V] and [K52R]). The remaining control lineage contained one prominent mutation in the 3′UTR of F, and the four missense mutations noted for the stressed lineage, except that in the case of this control, all four of these missense mutations in M2-1 were prominent. These four missense mutations in M2-1 were “t” to “c” mutations, suggesting that they were introduced on the genomic RNA by cellular deaminases.

Thus, both Min A-P[K27N] and Min A-P[F28V] exhibited substantially increased genetic stability compared to Min A. While Min A-P[F28V] still appeared to exhibit some residual level of instability in one lineage, the pattern was consistent with cytidine deaminase activity rather than polymerase infidelity; unlike for the parental virus Min A, no clear pattern of prominent instability emerged during the temperature stress test, showing that the introduction of these P mutations led to substantial improvements in stability.

Certain P mutations increased genomic RNA synthesis: The effect of P mutations [K25T], [K27N], [F28V] and [K32T] on Min A RNA synthesis was evaluated. These four mutations were selected as having a significant effect on increasing multicycle Min A replication at 37° C.

Single-cycle infections were performed in replicate monolayers of Vero cells in 6-well plates as previously described (Le Nouen et al. (2017)) to analyze in parallel the production of cell-associated viral RNAs, cell-associated viral proteins, and progeny virus. Cells were infected at a MOI of 3 PFU/cell at 37° C. with the indicated viruses. Two hours after infection, the cell monolayers were washed twice with PBS to remove the inoculum. Every 4 h from 4 to 24 h post-infection, 4 wells per virus were harvested. (i) One well was processed for cell-associated RNA for analysis by strand-specific RT-qPCR, as described below. (ii) Cells from a second well were harvested for analysis by flow cytometry, as described below. (iii) Another well was processed for cell lysates for Western blot analysis, as described below. (iv) Finally, the last well was used to harvest virus and determined the virus titer, as described above.

Infected Vero cells from single-cycle infections (MOI of 3 PFU/well, 37° C., described above) were harvested and the cell-associated RNA was collected using the RNeasy mini kit (Qiagen). RNA was subjected to strand-specific RT-qPCR to quantify viral negative-sense (genome) and positive-sense (mRNA and antigenome) RNA, as described previously (Le Nouen et al., P.N.A.S., 111(36): 13169-74 (2014)). Viral RNA was extracted using the RNeasy Mini Kit (Qiagen), and 5 μg of DNAse-treated RNA was reverse transcribed using SuperScript III First-Strand Synthesis System (Thermofisher) with first-strand primer specific either to genome or to antigenomic/mRNA and linked to an oligonucleotide tag (Le Nouen et al., 2017). Then, each cDNA was amplified in triplicate with a primer containing the oligonucleotide tag, a gene-specific reverse primer, and a probe. Strand-specificity was provided because only cDNAs containing the tagged RT primer sequence would be amplified. QPCR results were analyzed using the comparative threshold cycle (ΔCt) method, normalized to 18S rRNA internal control that had been subjected to RT-QPCR using random first-strand primers and a standard 18S rRNA Taqman assay (Thermofisher). Data were expressed as log 2 fold increase over the Min A 4-hour time point except for the quantification of wt NS1, NS2, N, P, M, and SH genes in wt RSV-infected cells that were expressed as fold increase over the wt 4-hour time point.

Vero cells were infected at 37° C. with an MOI of 3 PFU/cell wt RSV, Min A, or the Min A-derived viruses bearing the individual missense mutations. Replicate samples were collected in 4 h-intervals from 4 to 24 h to monitor viral gene expression, protein expression, RNA replication, and virus replication in a single-cycle infection experiment.

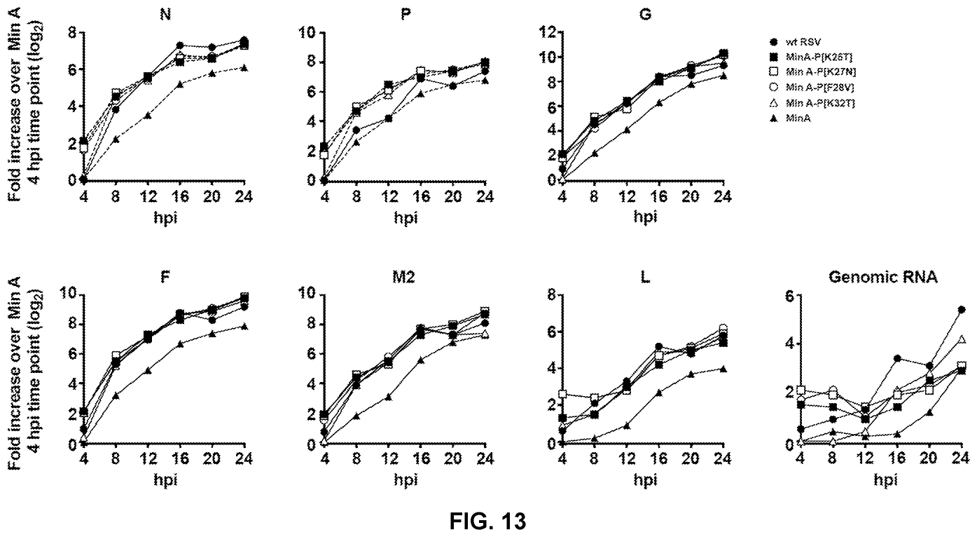

The level of transcription of each viral gene was evaluated by RT-qPCR assays, using tagged primers to specifically detect positive-sense RNA, which consists of mRNA and antigenomic RNA that typically are at a ratio of approximately 10:1 at the peak of RNA synthesis (FIG. 13). FIG. 13 shows quantification of N, P, G, F, M2 and L mRNA and antigenome by RT-qPCR using Taqman assays specific for each indicated ORF; for genome, quantification was by Taqman assay specific for the M2-1 ORF. S1 Fig shows quantification of NS1, NS2, N, P, M and SH mRNA and antigenome. Note that the quantification of the NS1, NS2, N, P, M and SH genes required different Taqman assays for Min A-derived viruses versus wt RSV because these ORFs were CPD in Min A-derivatives and wt in wt RSV. This precluded direct comparison of the Min A-derived viruses to wt RSV using these ORFs. although they are presented together in FIG. 13 (wt ORFs are indicated by solid lines and CPD ORFs by dashed lines). Conversely, the G, F, M2-1, and L ORFs were wt in all viruses and could be directly compared (FIG. 13).

Global viral RNA synthesis increased for all viruses over this time frame, and the increases seemed to level between 20 to 24 hpi (FIG. 13). When Min A and wt RSV were compared using the G, F, M2-1, and L ORFs that were identical in both viruses, the level of positive-sense RNA synthesis, reflecting mainly mRNA transcription, wt RSV was about three- to eight-fold above that of Min A at all time points, confirming our previous results (Le Nouen et al., PNAS USA, 111(36): 13169-74 (2014)). The insertion of each of the four P mutations increased the global mRNA levels of Min A (FIG. 13) to that observed for wt RSV, suggesting that each of these P mutations completely restored viral transcription of Min A. In case of the NS1, NS2, N, P, M and SH ORFs, although direct comparison to wt RSV was not possible, the magnitude of the increase compared to Min A was comparable to that observed for the G, F, M2, and L mRNAs (FIG. 13). Quantification of genomic RNA synthesis using tagged RT-qPCR specific for negative-strand RNA showed that the P mutations also increased the genomic RNA synthesis of Min A two to four-fold between 16 and 20 hpi.

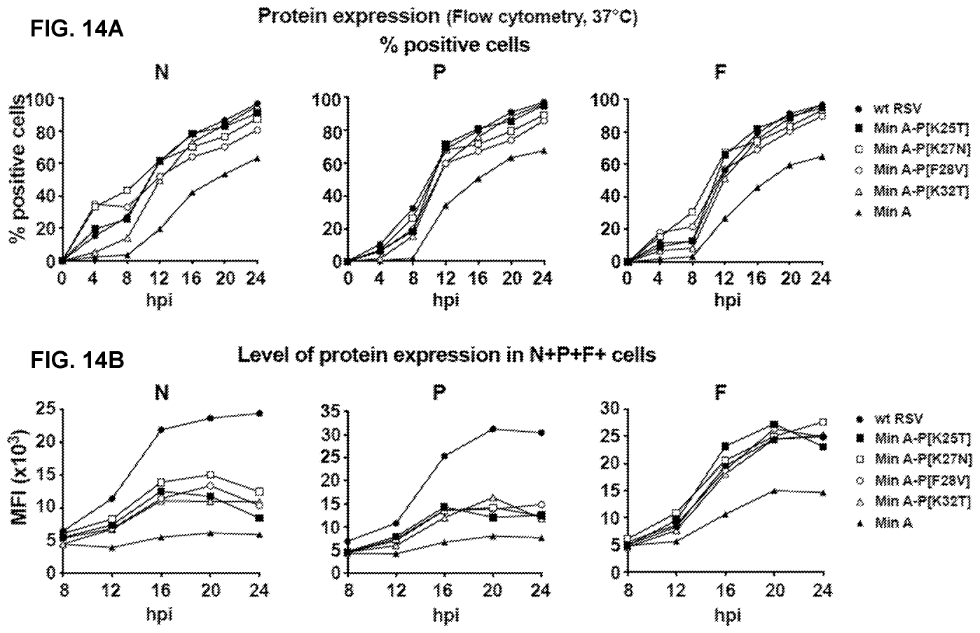

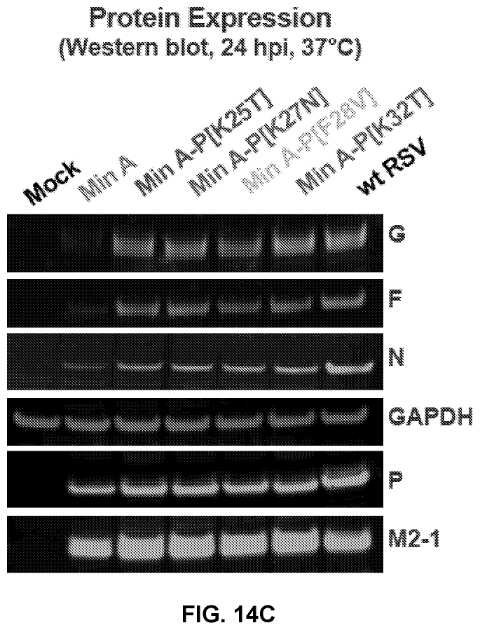

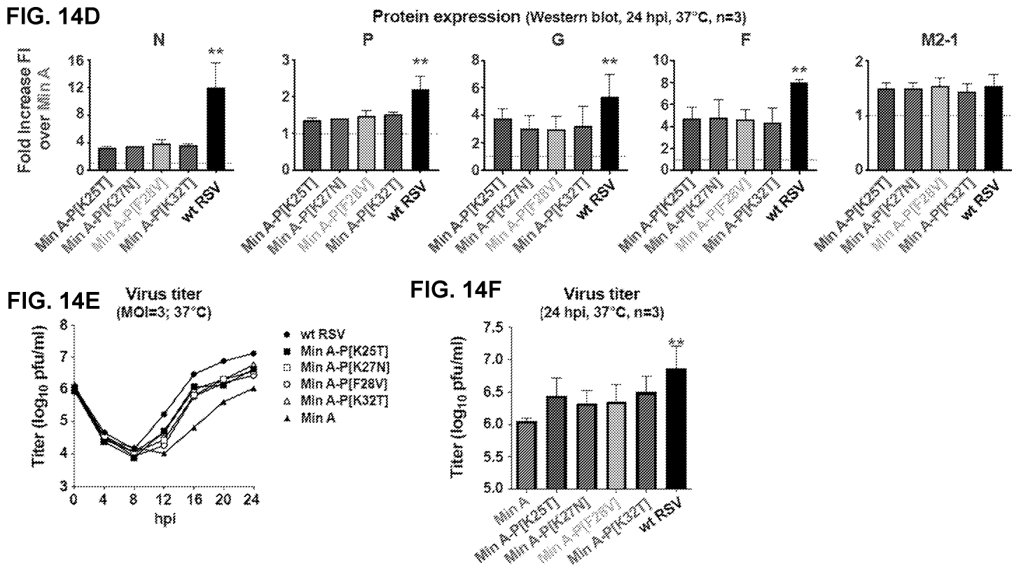

Certain P mutations increased Min A protein expression and virus replication: Also investigated was the level of cell-associated viral protein expression as well as virus replication in Vero cells from the same single-cycle infection experiment (MOI of 3 PFU/cell, 37° C.) that was described in FIG. 13. Infected cells were harvested at 4-h intervals from 4 to 24 hpi and viral protein expression was analyzed by flow cytometry (FIGS. 14A and B), and Western blotting (FIG. 14C; which is graphically represented in FIG. 7)) in replicate samples collected every 4 h from 4 to 24 hpi.

Regarding flow cytometry, infected Vero cells from single-cycle infections (MOI of 3 PFU/well, 37° C., described above) were harvested using TrypLE Select (Gibco) and stained with the pre-titered Live/Dead Fixable Near-IR Dead Cell dye (Thermofisher), followed by fixation and permeabilization using BD Cytofix/Cytoperm (BD Biosciences). Fixed and permeabilized cells in Perm/Wash buffer (BD Biosciences) were stained with a pre-titrated mixture of anti-RSV antibodies for the analysis of intracellular RSV protein expression: a fluorescein isothiocyanate (FITC)-labeled anti-RSV P MAb (Abcam), an allophycocyanin (APC)-labeled anti-RSV N MAb (Imgenex), and a Biotin-labeled anti-RSV F MAb (Millipore). Staining was performed for 30 minutes at room temperature in the dark. After incubation with the primary antibodies, cells were extensively washed with Perm/Wash Buffer and then incubated with a pre-titrated concentration of streptavidin-PE secondary antibody in the dark for 20 minutes at room temperature. After incubation, cells were washed extensively with Perm/Wash Buffer and resuspended in PBS. Live single cells were acquired using a BD flow cytometer Symphony (BD Biosciences). Data were analyzed using FlowJo 10.7. First, quality control of each acquired sample was performed using the FlowAI plugin that evaluates the flow rate, signal acquisition and dynamic range and removes cells with identified anomalies (Monaco et al., Bioinformatics, 32(16): 2473-80 (2016)). Then, compensation was performed automatically using single-color-labeled cells or beads for each antibody. Live/dead staining, forward scatter height, and forward scatter area were used to identify single live cells. Finally, the cell number was normalized to 19,000 across all samples using the DownSample plugin (FlowJo 10.7) and the expression of the virus proteins N, P, and F was analyzed on single live cells.

Regarding Western blot analysis, infected Vero cells from single-cycle infections (MOI of 3 PFU/well, 37° C., described above) were harvested in NuPage LDS sample buffer (Thermofisher) followed by homogenization using a QIAshredder spin column (Qiagen). Cell lysates were denatured at 90° C. for 10 min in 1× NuPAGE LDS Sample Buffer (Invitrogen) and 1× NuPAGE Sample Reducing Agent (Invitrogen) and subjected to electrophoresis in parallel with Odyssey Protein Molecular Weight Markers (Li-Cor) on NuPAGE 4-12% Bis-Tris Protein Gels (Thermofisher) with NuPAGE MES SDS Running Buffer (Life Technologies). Proteins were transferred to PVDF membranes using the iBlot 2 Gel Transfer Device (ThermoFisher). Membranes were blocked using Odyssey Blocking Buffer for one hour followed by overnight incubation with primary antibodies in Odyssey Blocking Buffer in PBS with 0.1% Tween 20 (Sigma-Aldrich). The primary antibodies were mouse MAbs against RSV N, P, M2-1 and G proteins (1:1,000, Abcam) and a rabbit polyclonal antibody preparation against GAPDH (1:200, Santa Cruz) as a loading control. The secondary antibodies used were goat anti-rabbit IgG IRDye 680, and goat anti-mouse IgG IRDye 800 (1:15000, Li-Cor). Membranes were scanned using Odyssey software, version 3.0 (Li-Cor). Fluorescence signals of the RSV proteins were background-corrected automatically by the Image Studio Lite software (Licor) and measured to quantify the intensity of each protein band. Values indicate the fluorescence intensity (FI) of each protein band.

Flow cytometry analysis showed that, for all of the mutant and control viruses, the percentage of cells that were positive for the N, P or F proteins increased steadily from 8 to 24 hpi (FIG. 14A). However, Min A infection seemed to progress at a slower rate: at 20 to 24 hpi, the percentage N-, P- or F-positive cells was about 2-fold lower than with wt RSV-infected cultures. In comparison, the percentage of positive cells for the Min A-derived viruses with P mutations was similar to that of wt RSV.

In addition, the level of expression (expressed as MFI) of N, P, and F protein in cells co-expressing all three proteins was investigated (FIG. 14D). The level of protein expression from the CPD N and P ORFs and the non-CPD F ORF in Min A derivatives containing the individual P mutations was increased by about two-fold compared to Min A. However, expression of N and P protein by the Min A-derived viruses with P mutations was still about two- to three-fold lower than for wt RSV, whereas the level of expression of F protein was restored to that of wt RSV. Thus, the individual P mutations restored mRNA transcription by Min A mutants to levels similar to wt RSV (as shown in FIG. 13), which also restored the level of expression of the non-CPD F protein to that of wt RSV. In contrast, expression of N and P proteins from the CPD ORFs remained reduced compared to wt RSV. This would be consistent with the paradigm that CPD reduces the efficiency of translation.

Additional replicate cultures from the single-cycle infection experiment (MOI of 3 PFU/cell, 37° C.) were harvested at 4-h intervals from 4 to 24 hpi and analyzed by Western blotting with antibodies specific to the G, F, N, P, and M2-1 proteins. A representative Western blot of the 24-h time point is shown in FIG. 14C. In addition, two additional repeats of the single-cycle infection experiment were performed in which infected cells were harvested at 24 hpi and subjected to the same Western blot analysis. These data were quantified together with those from FIG. 14C and are shown in FIG. 14D, expressed as fold-increase over Min A at 24 hpi. These results confirmed that the introduction of each of the P mutations into Min A increased the expression of the G, F, N, P, and M2-1 proteins. Specifically, the expression of N, G and F was increased by 3-5-fold compared to Min A, and the expression of P and M2-1 was increased by 1.3-1.5-fold. However, except for M2-1, the level of viral protein expression by the Min A-derived viruses containing individual P mutations remained lower compared to wt RSV. The finding that expression of G and F from non-CPD ORFs was not restored to wt levels when measured by Western blot (FIG. 14D) whereas expression of F was restored when measured by flow cytometry (FIG. 14B) might be because the latter method measured only infected cells and would be the more direct and relevant measure; Western blots measure all of the cells, and the somewhat-reduced efficiency of infection by the Min A-derived viruses compared to wt (FIG. 14A) might have contributed to a lower signal.

Also evaluated were the kinetics of virus replication from the same single-cycle infection experiment described herein. Thus, replicate infected Vero cell cultures (MOI 3 PFU/cell, 37° C.) were harvested at 4-h intervals from 4 to 24 hpi, and clarified cell-culture-medium supernatants were prepared and analyzed by immunoplaque assay to quantify infectious virus titers (FIG. 14E). Progeny viruses were first detected at approximately 12 hpi. At 24 hpi, wt RSV titers reached 107 PFU/ml, as typically observed. Min A replication was about 10-fold lower compared to wt RSV, whereas replication of the Min A derivatives bearing individual P mutations was approximately 5-fold higher than Min A but less than wt RSV (FIG. 14E). In addition, two additional repeats of the single-cycle infection experiment were performed in which infected cells were harvested at 24 and clarified culture medium supernatants were prepared and analyzed by immunoplaque assay, and the data were combined with that from FIG. 14E to create FIG. 14F. This confirmed that the individual P mutations increased Min A replication by about 5-fold, but not to the level of wt RSV.

Taken together, the viral mRNA levels in single-cycle replication experiments (FIG. 13) suggested that the P mutations restored Min A gene transcription to wt RSV level. However, the overall protein expression detected in infected Vero cells (FIGS. 14A-D) suggested that the translation of the CPD mRNAs still appeared to be reduced compared to wt mRNAs, thus resulting in reduced protein expression and reduced virus replication compared to wt RSV.

Animal experiments. All animal studies were approved by the NIH Institutional Animal Care and Use Committee (IACUC) under the animal study protocol number LID 34E. Replication of the CPD viruses was evaluated in the upper and lower respiratory tract of six-week old Golden Syrian hamsters.

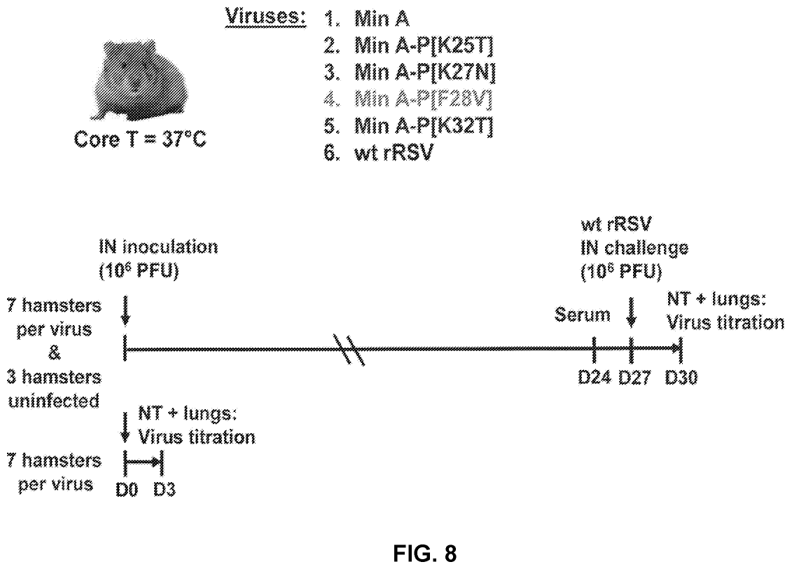

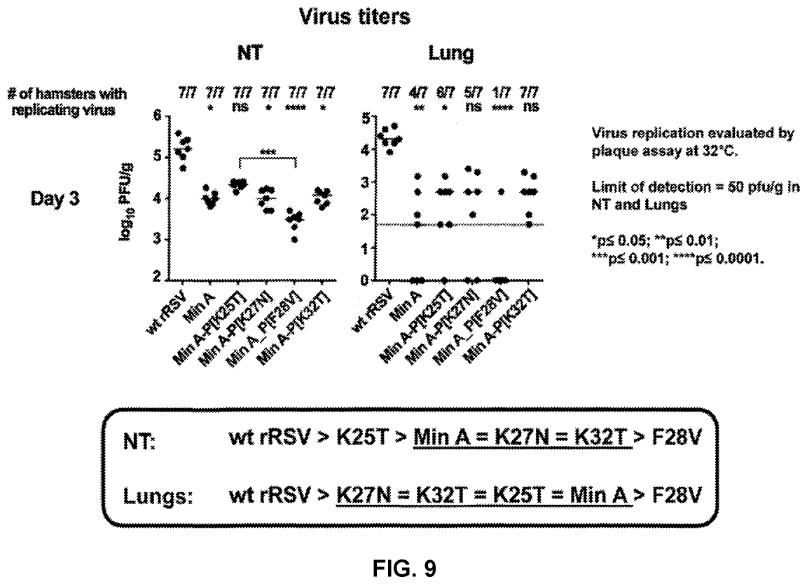

On day 0, groups of 14 hamsters were inoculated intranasally under isoflurane anesthesia with 10⁶ pfu of wt rRSV, Min A, Min A-P[K25T], Min A-P[K27N], Min A-P[F28V], or Min A-P[K32T]. Three additional hamsters were left uninfected as control. On day 3, which corresponds to the peak of replication of wt rRSV in hamsters (not shown), seven hamsters from each inoculated group were euthanized by carbon dioxide inhalation (see FIG. 8). Nasal turbinates (NT) and lungs were harvested and homogenized separately in Leibovitz (L-15) Medium containing 2% L-glutamine, 1% Amphotericin B, 0.1% Gentamicin, and 0.06 mg/mL clindamycin phosphate. Virus titers were determined in duplicate by plaque assay on Vero cells incubated in 32° C. The limit of virus detection was 50 pfu/g in both the NT and lungs (see FIG. 9).

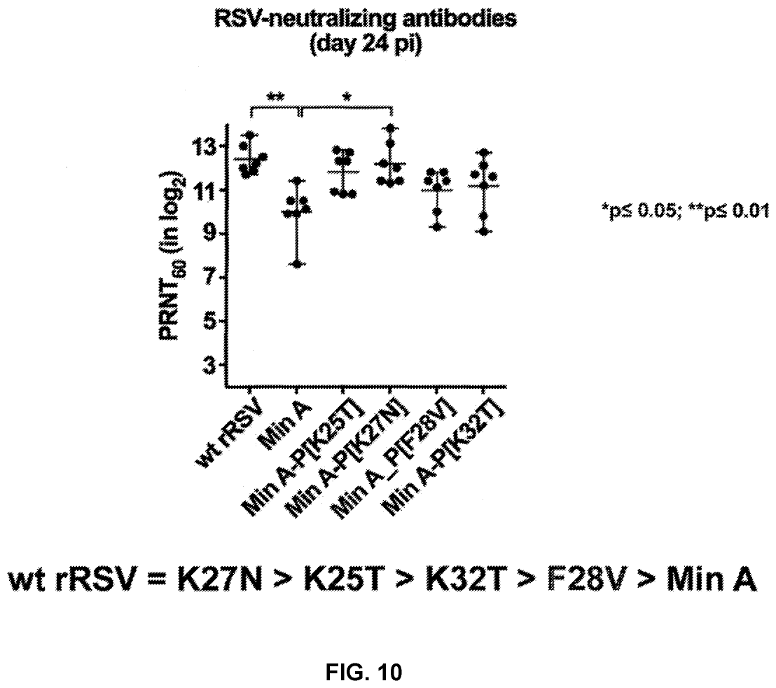

Immunogenicity of CPD viruses was also tested. Serum was collected from the blood of seven hamsters per group the day prior to immunization and at day 24 post-immunization to measure the RSV antibody response. The 60% plaque reduction neutralizing antibody titers (PRNT60) were determined as previously described (Le Nouen et al. (2014), supra) (see FIG. 10).

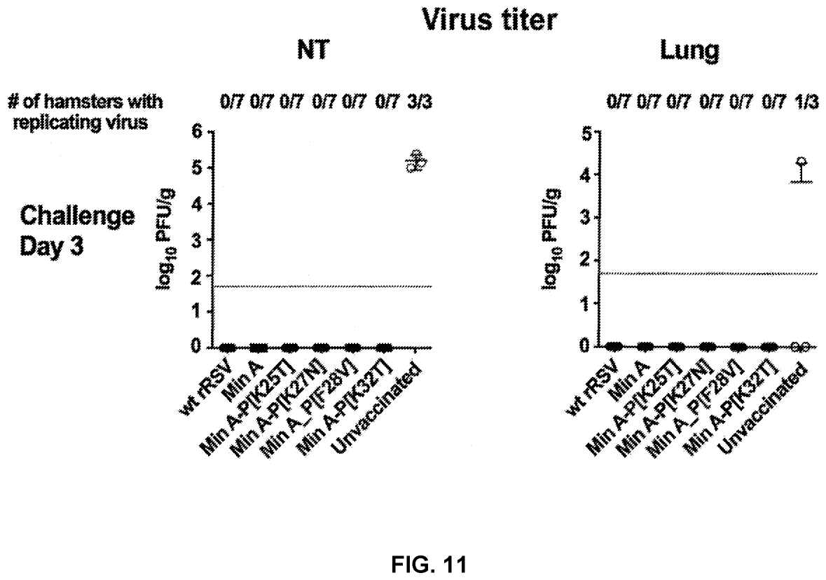

On day 27 post-immunization, the remaining hamsters were challenged with 10⁶ pfu of wt rRSV via intranasal administration (see FIG. 11). Three days after challenge, hamsters were euthanized by carbon dioxide inhalation. NT and lung tissue were harvested and wt rRSV virus titers were determined in duplicate by plaque assay on Vero cells incubated at 32° C. as described above. Statistical differences compared with wt rRSV is indicated on the top of each graph while statistical differences between Min A and the Min A-derived mutants are indicated by brackets (*p≤0.05, **p≤0.01, and ****p≤0.0001).

### Example 2

Generation and Analysis of Certain Additional Mutants: Several additional prominent mutations that had co-emerged with P[K27N] (in lineage #4) and P[F28V] (lineage #5) during the serial passages of Min A (Table 1) were re-introduced into Min A-P[K27N] and Min A-P[F28V] in accordance with the method described in Example 1.

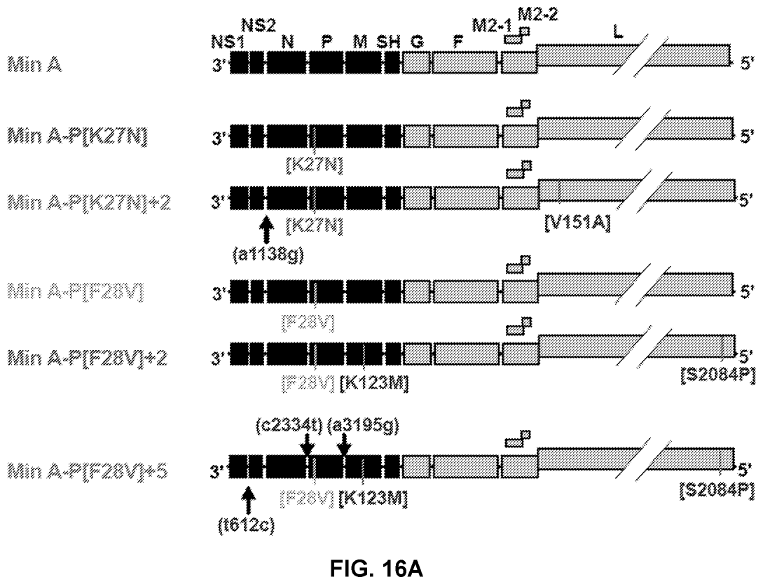

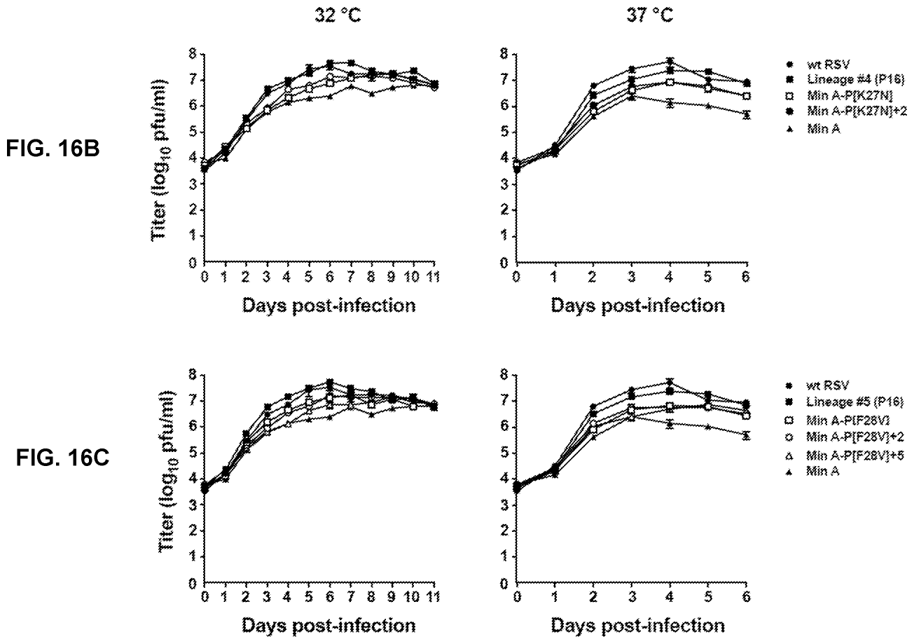

In addition to P[K27N], lineage #4 had accumulated four other prominent mutations (Table 1): one was in the 5′ UTR of the N gene, specifically a1138g in the Kozak sequence (Kozak, M, Nucleic Acids Res., 15(20): 8125-48 (1987)) at position −3 preceding the translation initiation codon; a second was the missense mutation [V151A] in the L ORF; and the remaining two were in ORFs but were silent and thus predicted to be inconsequential for attenuation and immunogenicity. Mutations a1138g and L[V151A] were introduced into Min A-P[K27N] backbone to generate the virus Min A-P[K27N]+2 (FIG. 16A).

In addition to P[F28V], lineage #5 had accumulated five other prominent mutations (Table 1): two were non-synonymous in M ([K123M]) and L ([S2084P]) and three were non-coding, occurring in the NS2 5′UTR (t612c), the P gene-start signal (c2334t, GGGGCAAAT), and the P 3′UTR (a3195g). Note that this nucleotide substitution in the gene-start signal had no detectable effect on transcription in a mini-genome system. Five mutations were introduced into Min A-P[F28V] backbone by reverse genetics in two combinations: (i) the two non-synonymous mutations in M ([K123M]) and L ([S2084P]) in addition to the P[F28V] mutation, resulting in the virus Min A-P[F28V]+2, and (ii) all five prominent mutations in addition to the P[F28V] mutation, resulting in Min A-P[F28V]+5 (FIG. 16A). The sequence of each virus was confirmed by Sanger sequencing.

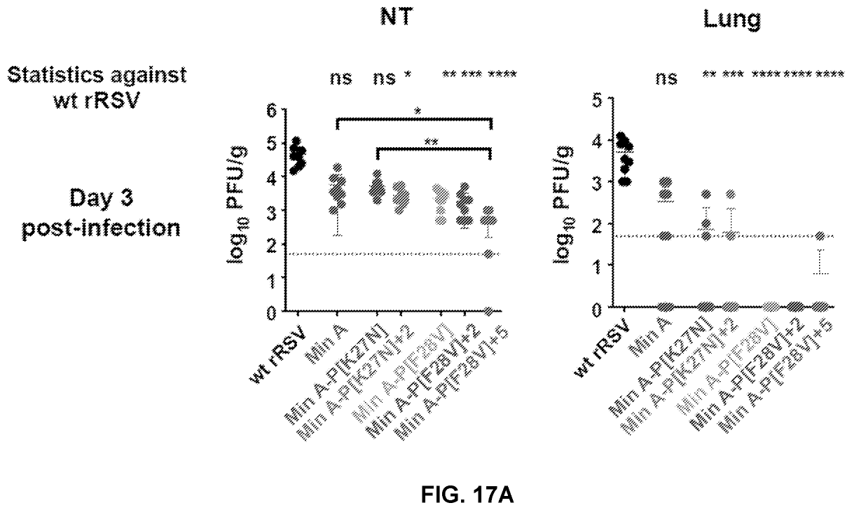

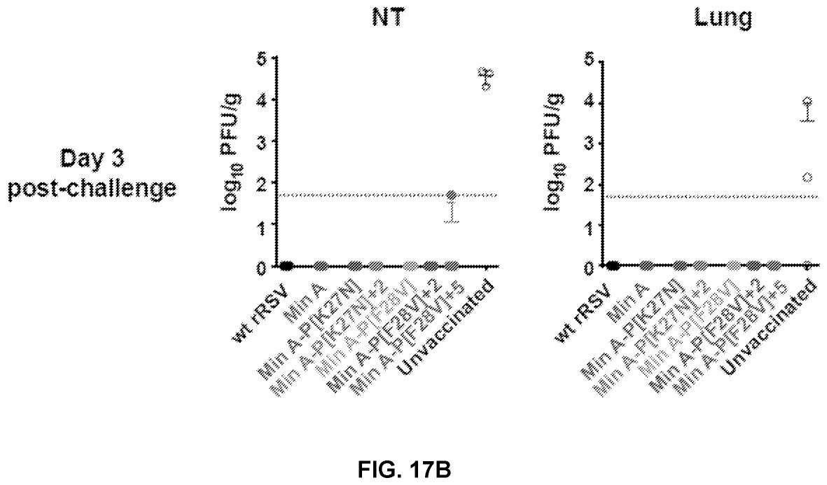

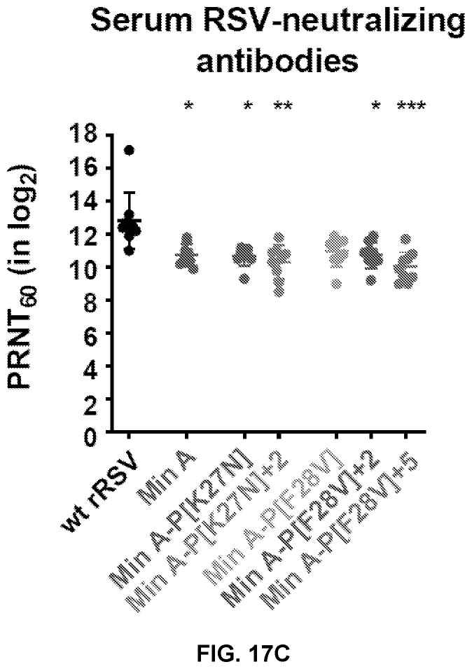

Multicycle growth kinetic: Evaluation of these viruses in a multicycle replication experiment in Vero cells incubated at 32° C. or 37° C. showed that the introduction of these additional mutations into Min A-P[K27N] and Min A-P[F28V] did not further improve the replication of these viruses (FIGS. 16B-C)). In addition, the presence of the additional missense mutations did not significantly alter the replication and immunogenicity of the Min A-derived P mutants in hamsters (FIG. 17), except for Min A-P[F28V]+5, which exhibited a slightly reduced replication and immunogenicity compared to Min A-P[F28V].

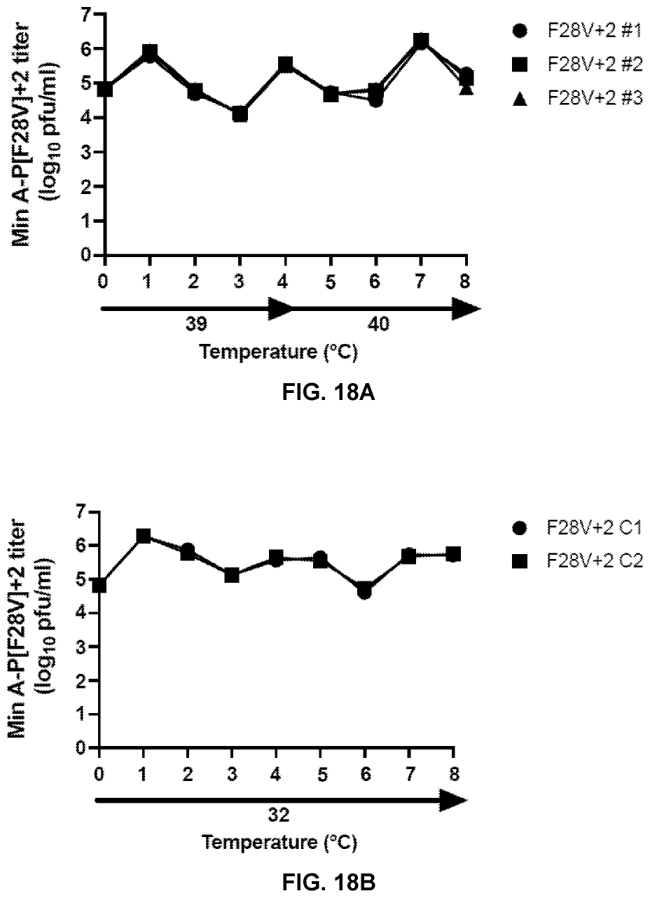

Genetic stability: The genetic stability of Min A-P[F28V]+2 was evaluated in a temperature stress test involving four passages at 39° C. and four passages at 40° C., corresponding to 2 months of continuous passage (FIG. 18A). This virus was also passaged in parallel at the permissive temperature of 32° C. as a control (FIG. 18B). Only three subdominant missense mutations were found in the five lineages at the end of the stress test. Thus, Min A-P[F28V]+2 exhibited a further increased genetic stability compared to Min A and Min A-P[F28V].

Amino acids may be referred to herein by either the commonly known three letter symbols or by the one-letter symbols recommended by the IUPAC-IUB Biochemical Nomenclature Commission.

The terms “about” and “around,” as used herein to modify a numerical value, indicate a close range surrounding the numerical value. Thus, if “X” is the value, “about X” or “around X” indicates a value of from 0.9× to 1.1×, e.g., from 0.95× to 1.05× or from 0.99× to 1.01×. A reference to “about X” or “around X” specifically indicates at least the values X, 0.95×, 0.96×, 0.97×, 0.98×, 0.99×, 1.01×, 1.02×, 1.03×, 1.04×, and 1.05×. Accordingly, “about X” and “around X” are intended to teach and provide written description support for a claim limitation of, e.g., “0.98×.”

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A polynucleotide encoding a recombinant respiratory syncytial virus (RSV) variant having an attenuated phenotype comprising a modified RSV genome or antigenome that encodes a mutant RSV protein P that differs from a parental RSV protein P at one or more amino acid residues.

2. The polynucleotide of claim 1, wherein the mutant RSV protein P comprises an amino acid sequence that differs from the amino acid sequence set forth in SEQ ID NO: 6 at one or more positions selected from the group consisting of 19-34, 107, 229, 234, and 235.

3. The polynucleotide of claim 1, wherein the mutant RSV protein comprises an amino acid sequence that differs from the amino acid sequence set forth in SEQ ID NO: 6 at at least position 25, 27, 28, 32, 34, or any combination thereof.

4. The polynucleotide of claim 3, wherein the residue at position 25 of the amino acid sequence is threonine or asparagine, the residue at position 27 of the amino acid sequence is glutamic acid or asparagine, the residue at position 28 of the amino acid sequence is valine, isoleucine, proline, leucine, or serine, the residue at position 32 of the amino acid sequence is threonine, the residue at position 34 of the amino acid sequence is serine, or any combination thereof.

5. The polynucleotide of claim 1, wherein the mutant RSV protein has comprises an amino acid sequence that differs from the amino acid sequence set forth in SEQ ID NO: 6 at at least position 25.

6. The polynucleotide of claim 1, wherein the mutant RSV protein comprises an amino acid sequence that differs from the amino acid sequence set forth in SEQ ID NO: 6 at at least position 27.

7. The polynucleotide of claim 1, wherein the mutant RSV protein comprises an amino acid sequence that differs from the amino acid sequence set forth in SEQ ID NO: 6 at at least position 28.

8. (canceled)

9. The polynucleotide of claim 1, wherein the mutant RSV protein comprises an amino acid sequence that differs from the amino acid sequence set forth in SEQ ID NO: 6 at at least position 32.

10. (canceled)

11. The polynucleotide of claim 1, wherein the mutant RSV protein comprises an amino acid sequence that differs from the amino acid sequence set forth in SEQ ID NO: 6 at at least position 34.

12. (canceled)

13. The polynucleotide of claim 1, wherein one or more ORFs of the modified RSV genome or antigenome is codon-pair deoptimized.

14. The polynucleotide of claim 1, wherein the nucleotide sequence of the modified RSV genome or antigenome encoding one or more of RSV proteins NS1, NS2, N, P, M, and SH has about 70% to about 95% identity with the nucleotide sequence of the parental RSV genome or antigenome encoding the same one or more of RSV proteins NS1, NS2, N, P, M, and SH.

15-32. (canceled)

33. The polynucleotide of claim 14, wherein the amino acid sequence of the one or more of RSV proteins NS1, NS2, N, P, M, and SH encoded by the nucleotide sequence of the modified RSV genome or antigenome is at least 99% identical to the amino acid sequence of the same one or more of RSV proteins NS1, NS2, N, P, M, and SH encoded by the nucleotide sequence of the parental RSV genome or antigenome.

34. A recombinant RSV variant comprising the polynucleotide of claim 1.

35. A pharmaceutical composition comprising the recombinant RSV variant of claim 34 and at least one excipient.

36. A multivalent RSV vaccine composition comprising at least two recombinant RSV variants of claim 34, wherein each recombinant RSV variant has a different nucleotide sequence.

37. A method of vaccinating an animal, comprising administering the pharmaceutical composition of claim 35 to an animal.

38. A method of inducing an immune response in an animal, comprising administering the recombinant RSV variant of claim 34 to an animal.

39. The method of claim 38, wherein the recombinant RSV variant is administered via injection, nasal spray, nasal droplets, topical application, aerosol delivery, or oral inoculation.

40. The method of claim 37, wherein the animal is a mammal.

41. (canceled)

42. A method of producing a recombinant RSV variant vaccine, comprising expressing the polynucleotide of claim 1 in a cell.


SEQUENCE LISTING (24 sequences)

SEQ ID NO: 1 - DNA, 15111 bp, Human respiratory syncytial virus
acgggaaaaaatgcgtacaacaaacttgcataaaccaaaaaaatggggcaaataagaatttgataagtaccacttaaatttaactcccttggttagagatgggcagcaattcattgagtatgataaaagttagattacaaaatttgtttgacaatgatgaagtagcattgttaaaaataacatgctatactgataaattaatacatttaactaatgctttggctaaggcagtgatacatacaatcaaattgaatggcattgtgtttgtgcatgttattacaagtagtgatatttgccctaataataatattgtagtaaaatccaatttcacaacaatgccagtactacaaaatggaggttatatatgggaaatgatggaattaacacattgctctcaacctaatggtctactagatgacaattgtgaaattaaattctccaaaaaactaagtgattcaacaatgaccaattatatgaatcaattatctgaattacttggatttgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgatcacagacatgagaccgttgtcacttgagaccataataacatcactaaccagagacatcataacacacaaatttatatacttgataaatcatgaatgcatagtgagaaaacttgatgaaagacaggccacatttacattcctggtcaactatgaaatgaaactattacacaaagtaggaagcactaaatataaaaaatatactgaatacaacacaaaatatggcactttccctatgccaatattcatcaatcatgatgggttcttagaatgcattggcattaagcctacaaagcatactcccataatatacaagtatgatctcaatccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcggttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttgagttaataaaaaatggggcaaataaatcatcatggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatggttggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggctccacatacacagctgctgttcaatacaatgtcttagaaaaagacgatgaccctgcatcacttacaatatgggtgcccatgttccaatcatctatgccagcagatttacttataaaagaactagctaatgtcaacatactagtgaaacaaatatccacacccaagggaccttcactaagagtcatgataaactcaagaagtgcagtgctagcacaaatgcccagcaaatttaccatatgcgctaatgtgtccttggatgaaagaagcaaactagcatatgatgtaaccacaccctgtgaaatcaaggcatgtagtctaacatgcctaaaatcaaaaaatatgttgactacagttaaagatctcactatgaagacactcaaccctacacatgatattattgctttatgtgaatttgaaaacatagtaacatcaaaaaaagtcataataccaacatacctaagatccatcagtgtcagaaataaagatctgaacacacttgaaaatataacaaccactgaattcaaaaatgctatcacaaatgcaaaaatcatcccttactcaggattactattagtcatcacagtgactgacaacaaaggagcattcaaatacataaagccacaaagtcaattcatagtagatcttggagcttacctagaaaaagaaagtatatattatgttaccacaaattggaagcacacagctacacgatttgcaatcaaacccatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagttaatacttgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 2 - DNA, 15111 bp, Human respiratory syncytial virus
acgggaaaaaatgcgtacaacaaacttgcataaaccaaaaaaatggggcaaataagaatttgataagtaccacttaaatttaactcccttggttagagatgggcagcaattcattgagtatgataaaagtcagattgcaaaatctattcgataatgacgaagtggcactattaaaaattacatgttataccgataaattgatacatctaactaatgcattagctaaagctgtaatacatacaattaaacttaatggaatagtgtttgtacatgtaattacatctagtgatatatgccctaataataatatcgtagtcaagtctaattttacaacaatgccagtgttacaaaatggcggatatatttgggaaatgatggaattgacacattgctcacaacctaatggtctattagacgataattgcgaaattaaatttagtaagaaattatccgatagtacaatgactaattatatgaatcaattatccgaattgttaggtttcgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgattaccgatatgagaccgttgtcacttgagacaattataactagcctaactagagatataataacacataaatttatatatctgattaatcacgaatgcatcgtgaggaaattggacgaaagacaggccacatttacattcttagtcaattacgaaatgaaactattgcataaggtaggctcaactaagtataagaaatataccgaatataacactaaatacggaacattcccaatgcctatattcataaatcacgacgggtttctcgaatgcataggcataaaacctacaaaacatacacccataatctataaatacgatcttaacccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacattgaataaagatcaattactatctagctcgaaatatactatccaacggtctacaggcgattcaatagatacacctaattacgatgtgcaaaaacatattaataaattgtgtggtatgttattgattaccgaagacgcaaatcataaatttacagggttaatcggtatgttatacgctatgtctagattaggtagggaagatacaattaaaatacttagagacgcaggatatcacgttaaagctaacggagtagacgtaactacacatagacaggatattaacggtaaggaaatgaaattcgaagtgttaacactcgctagcttaactaccgaaatacaaattaatatcgaaatcgaatcacgtaaatcttataagaaaatgcttaaagaaatgggcgaagtcgcacccgaatatagacacgatagtcccgattgtggtatgattatactatgtatagccgcattagtgataactaagttggccgcaggcgatagatccggattaaccgcagtgatacgtagagcgaataacgtacttaaaaacgaaatgaaacggtataagggtctattaccaaaagatatagcgaatagtttttacgaagtattcgaaaaacatccacattttatagacgtttttgtgcatttcggaatcgcacaatctagtactagaggaggatctagggttgagggtatattcgcaggattgtttatgaacgcatacggagcaggtcaagtcatgcttagatggggagtactcgcaaaatccgttaaaaatattatgttaggacacgctagcgtacaagccgaaatggaacaagtcgttgaggtatacgaatacgcacaaaaattaggtggagaagcaggattttatcatatactgaataatcctaaagctagtctattaagcttaacacaatttccacatttttctagcgtagtgttaggtaacgcagctggcctaggcataatgggcgaatataggggtacacctagaaatcaggatctatatgacgcagctaaagcatacgctgaacaattgaaagagaatggagtgataaattattccgtactcgatctaacagccgaagagttggaggcaattaaacatcaattgaatccgaaagataatgacgttgagttgtgagttaataaaaaatggggcaaataaatcatcatggaaaagtttgctcctgaattccatggagaagacgcaaataatagggcaacaaaattcttagagtcaatcaagggtaagtttacaagtccaaaagatccaaagaagaaagatagtataataagcgtaaactcaattgatatcgaggtgacaaaggaatcacctataacatctaatagtacaataataaatcccactaacgaaacagacgataccgcaggcaataaacctaattatcaacggaaacccttagtgtcattcaaagaagatccaacacctagtgataatccctttagtaaattgtataaggaaacaatcgaaacattcgataataacgaagaagaatcatcatactcatacgaagagataaacgatcagactaacgataatataaccgctagactagatagaatagacgaaaaactatctgaaatactaggtatgttacacacactagtagtcgcatctgccggacctacaagtgctagagatgggataagggatgcaatggtagggttaagggaagaaatgatagagaaaattagaaccgaagcattaatgactaacgatagactcgaagcaatggctagacttagaaacgaagaatccgaaaagatggcaaaagatacatctgacgaagtgtcacttaatcctactagcgaaaaattgaataatctattagagggaaacgatagtgataacgatctatcactcgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggatcaacatatacagctgcagtccaatataacgtactcgaaaaagacgacgatcccgctagcctaacaatatgggtcccaatgtttcaatctagtatgcccgctgatctattaatcaaagaactagctaacgttaacatactagtcaaacaaattagtacacctaagggaccctcacttagagtgatgattaatagtagatccgcagtcctagcacaaatgcctagtaagtttacaatatgtgctaacgtaagcttagacgaacgatcaaaactagcatacgatgtgacaacaccatgcgaaatcaaagcatgttcattgacatgtcttaaatcaaagaatatgctaacaacagtcaaagatctaacaatgaaaacacttaatcccacacacgatataatcgcactatgcgaattcgaaaatatagtgactagtaagaaagtgataatccctacataccttagatcaatatccgttagaaataaggatctgaatacactcgaaaatataacaacaaccgaattcaaaaacgctataactaacgctaagataatcccttactccggactattgttagtgataaccgtaaccgataataagggagcattcaaatacataaaaccccaatcccaatttatagtcgatttaggcgcatacttagaaaaagaatcaatctattacgttacaactaattggaaacataccgctactagattcgcaatcaaacctatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctctagcaaattttggccttactttacactaatacacatgataactacaatcatatccctattaatcataatctcaattatgatcgcaatccttaacaaactatgtgagtataacgtattccataacaaaacattcgaattgccaagagctcgagtgaatacctgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 3 - protein, 139 aa, respiratory syncytial virus
MGSNSLSMIKVRLQNLFDNDEVALLKITCYTDKLIHLTNALAKAVIHTIKLNGIVFVHVITSSDICPNNNIVVKSNFTTMPVLQNGGYIWEMMELTHCSQPNGLLDDNCEIKFSKKLSDSTMTNYMNQLSELLGFDLNP

SEQ ID NO: 4 - protein, 124 aa, respiratory syncytial virus
MDTTHNDNTPQRLMITDMRPLSLETIITSLTRDIITHKFIYLINHECIVRKLDERQATFTFLVNYEMKLLHKVGSTKYKKYTEYNTKYGTFPMPIFINHDGFLECIGIKPTKHTPIIYKYDLNP

SEQ ID NO: 5 - protein, 391 aa, respiratory syncytial virus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 6 - protein, 241 aa, respiratory syncytial virus
MEKFAPEFHGEDANNRATKFLESIKGKFTSPKDPKKKDSIISVNSIDIEVTKESPITSNSTIINPTNETDDTAGNKPNYQRKPLVSFKEDPTPSDNPFSKLYKETIETFDNNEEESSYSYEEINDQTNDNITARLDRIDEKLSEILGMLHTLVVASAGPTSARDGIRDAMVGLREEMIEKIRTEALMTNDRLEAMARLRNEESEKMAKDTSDEVSLNPTSEKLNNLLEGNDSDNDLSLEDF

SEQ ID NO: 7 - protein, 256 aa, respiratory syncytial virus
METYVNKLHEGSTYTAAVQYNVLEKDDDPASLTIWVPMFQSSMPADLLIKELANVNILVKQISTPKGPSLRVMINSRSAVLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 8 - protein, 64 aa, respiratory syncytial virus
MENTSITIEFSSKFWPYFTLIHMITTIISLLIIISIMIAILNKLCEYNVFHNKTFELPRARVNT

SEQ ID NO: 9 - protein, 298 aa, respiratory syncytial virus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 10 - protein, 574 aa, respiratory syncytial virus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 11 - protein, 194 aa, respiratory syncytial virus
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTVSDTNDHAKNNDTT

SEQ ID NO: 12 - protein, 90 aa, respiratory syncytial virus
MTMPKIMILPDKYPCSITSILITSRCRVTMYNQKNTLYFNQNNPNNHMYSPNQTFNEIHWTSQELIDTIQNFLQHLGIIEDIYTIYILVS

SEQ ID NO: 13 - protein, 2165 aa, respiratory syncytial virus
MDPIINGNSANVYLTDSYLKGVISFSECNALGSYIFNGPYLKNDYTNLISRQNPLIEHMNLKKLNITQSLISKYHKGEIKLEEPTYFQSLLMTYKSMTSSEQIATTNLLKKIIRRAIEISDVKVYAILNKLGLKEKDKIKSNNGQDEDNSVITTIIKDDILSAVKDNQSHLKADKNHSTKQKDTIKTTLLKKLMCSMQHPPSWLIHWFNLYTKLNNILTQYRSNEVKNHGFTLIDNQTLSGFQFILNQYGCIVYHKELKRITVTTYNQFLTWKDISLSRLNVCLITWISNCLNTLNKSLGLRCGFNNVILTQLFLYGDCILKLFHNEGFYIIKEVEGFIMSLILNITEEDQFRKRFYNSMLNNITDAANKAQKNLLSRVCHTLLDKTVSDNIINGRWIILLSKFLKLIKLAGDNNLNNLSELYFLFRIFGHPMVDERQAMDAVKINCNETKFYLLSSLSMLRGAFIYRIIKGFVNNYNRWPTLRNAIVLPLRWLTYYKLNTYPSLLELTERDLIVLSGLRFYREFRLPKKVDLEMIINDKAISPPKNLIWTSFPRNYMPSHIQNYIEHEKLKFSESDKSRRVLEYYLRDNKFNECDLYNCVVNQSYLNNPNHVVSLTGKERELSVGRMFAMQPGMFRQVQILAEKMIAENILQFFPESLTRYGDLELQKILELKAGISNKSNRYNDNYNNYISKCSIITDLSKFNQAFRYETSCICSDVLDELHGVQSLFSWLHLTIPHVTIICTYRHAPPYIGDHIVDLNNVDEQSGLYRYHMGGIEGWCQKLWTIEAISLLDLISLKGKFSITALINGDNQSIDISKPIRLMEGQTHAQADYLLALNSLKLLYKEYAGIGHKLKGTETYISRDMQFMSKTIQHNGVYYPASIKKVLRVGPWINTILDDFKVSLESIGSLTQELEYRGESLLCSLIFRNVWLYNQIALQLKNHALCNNKLYLDILKVLKHLKTFFNLDNIDTALTLYMNLPMLFGGGDPNLLYRSFYRRTPDFLTEAIVHSVFILSYYTNHDLKDKLQDLSDDRLNKFLTCIITFDKNPNAEFVTLMRDPQALGSERQAKITSEINRLAVTEVLSTAPNKIFSKSAQHYTTTEIDLNDIMQNIEPTYPHGLRVVYESLPFYKAEKIVNLISGTKSITNILEKTSAIDLTDIDRATEMMRKNITLLIRILPLDCNRDKREILSMENLSITELSKYVRERSWSLSNIVGVTSPSIMYTMDIKYTTSTISSGIIIEKYNVNSLTRGERGPTKPWVGSSTQEKKTMPVYNRQVLTKKQRDQIDLLAKLDWVYASIDNKDEFMEELSIGTLGLTYEKAKKLFPQYLSVNYLHRLTVSSRPCEFPASIPAYRTTNYHFDTSPINRILTEKYGDEDIDIVFQNCISFGLSLMSVVEQFTNVCPNRIILIPKLNEIHLMKPPIFTGDVDIHKLKQVIQKQHMFLPDKISLTQYVELFLSNKTLKSGSHVNSNLILAHKISDYFHNTYILSTNLAGHWILIIQLMKDSKGIFEKDWGEGYITDHMFINLKVFFNAYKTYLLCFHKGYGKAKLECDMNTSDLLCVLELIDSSYWKSMSKVFLEQKVIKYILSQDASLHRVKGCHSFKLWFLKRLNVAEFTVCPWVVNIDYHPTHMKAILTYIDLVRMGLINIDRIHIKNKHKFNDEFYTSNLFYINYNFSDNTHLLTKHIRIANSELENNYNKLYHPTPETLENILANPIKSNDKKTLNDYCIGKNVDSIMLPLLSNKKLIKSSAMIRTNYSKQDLYNLFPMVVIDRIIDHSGNTAKSNQLYTTTSHQISLVHNSTSLYCMLPWHHINRFNFVFSSTGCKISIEYILKDLKIKDPNCIAFIGEGAGNLLLRTVVELHPDIRYIYRSLKDCNDHSLPIEFLRLYNGHINIDYGENLTIPATDATNNIHWSYLHIKFAEPISLFVCDAELSVTVNWSKIIIEWSKHVRKCKYCSSVNKCMLIVKYHAQDDIDFKLDNITILKTYVCLGSKLKGSEVYLVLTIGPANIFPVFNVVQNAKLILSRTKNFIMPKKADKESIDANIKSLIPFLCYPITKKGINTALSKLKSVVSGDILSYSIAGRNEVFSNKLINHKHMNILKWFNHVLNFRSTELNYNHLYMVESTYPYLSELLNSLTTNELKKLIKITGSLLYNFHNE

SEQ ID NO: 14 - DNA, 10 bp, respiratory syncytial virus
ggggcaaata

SEQ ID NO: 15 - DNA, 10 bp, respiratory syncytial virus
ggggcaaaca

SEQ ID NO: 16 - DNA, 23 bp, respiratory syncytial virus
aaatcaactcagccaacccaacc

SEQ ID NO: 17 - DNA, 6 bp, respiratory syncytial virus
acagcc

SEQ ID NO: 18 - DNA, 17 bp, respiratory syncytial virus
ggggtaaataaatcatc

SEQ ID NO: 19 - DNA, 158 bp, respiratory syncytial virus
ttagttaccaatcttcacatcaacacacaataccaacagaagaccaacaaactaaccaacccaatcatccaaccaaacatccatccgccaatcagccaaacagccaacaaaacaaccagccagtccaaaactaaccacccggaaaaaatctataatat

SEQ ID NO: 20 - DNA, 15111 bp, respiratory syncytial virus
acgggaaaaaatgcgtacaacaaacttgcataaaccaaaaaaatggggcaaataagaatttgataagtaccacttaaatttaactcccttggttagagatgggcagcaattcattgagtatgataaaagtcagattgcaaaatctattcgataatgacgaagtggcactattaaaaattacatgttataccgataaattgatacatctaactaatgcattagctaaagctgtaatacatacaattaaacttaatggaatagtgtttgtacatgtaattacatctagtgatatatgccctaataataatatcgtagtcaagtctaattttacaacaatgccagtgttacaaaatggcggatatatttgggaaatgatggaattgacacattgctcacaacctaatggtctattagacgataattgcgaaattaaatttagtaagaaattatccgatagtacaatgactaattatatgaatcaattatccgaattgttaggtttcgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgattaccgatatgagaccgttgtcacttgagacaattataactagcctaactagagatataataacacataaatttatatatctgattaatcacgaatgcatcgtgaggaaattggacgaaagacaggccacatttacattcttagtcaattacgaaatgaaactattgcataaggtaggctcaactaagtataagaaatataccgaatataacactaaatacggaacattcccaatgcctatattcataaatcacgacgggtttctcgaatgcataggcataaaacctacaaaacatacacccataatctataaatacgatcttaacccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacattgaataaagatcaattactatctagctcgaaatatactatccaacggtctacaggcgattcaatagatacacctaattacgatgtgcaaaaacatattaataaattgtgtggtatgttattgattaccgaagacgcaaatcataaatttacagggttaatcggtatgttatacgctatgtctagattaggtagggaagatacaattaaaatacttagagacgcaggatatcacgttaaagctaacggagtagacgtaactacacatagacaggatattaacggtaaggaaatgaaattcgaagtgttaacactcgctagcttaactaccgaaatacaaattaatatcgaaatcgaatcacgtaaatcttataagaaaatgcttaaagaaatgggcgaagtcgcacccgaatatagacacgatagtcccgattgtggtatgattatactatgtatagccgcattagtgataactaagttggccgcaggcgatagatccggattaaccgcagtgatacgtagagcgaataacgtacttaaaaacgaaatgaaacggtataagggtctattaccaaaagatatagcgaatagtttttacgaagtattcgaaaaacatccacattttatagacgtttttgtgcatttcggaatcgcacaatctagtactagaggaggatctagggttgagggtatattcgcaggattgtttatgaacgcatacggagcaggtcaagtcatgcttagatggggagtactcgcaaaatccgttaaaaatattatgttaggacacgctagcgtacaagccgaaatggaacaagtcgttgaggtatacgaatacgcacaaaaattaggtggagaagcaggattttatcatatactgaataatcctaaagctagtctattaagcttaacacaatttccacatttttctagcgtagtgttaggtaacgcagctggcctaggcataatgggcgaatataggggtacacctagaaatcaggatctatatgacgcagctaaagcatacgctgaacaattgaaagagaatggagtgataaattattccgtactcgatctaacagccgaagagttggaggcaattaaacatcaattgaatccgaaagataatgacgttgagttgtgagttaataaaaaatggggcaaataaatcatcatggaaaagtttgctcctgaattccatggagaagacgcaaataatagggcaacaaaattcttagagtcaatcaagggtaattttacaagtccaaaagatccaaagaagaaagatagtataataagcgtaaactcaattgatatcgaggtgacaaaggaatcacctataacatctaatagtacaataataaatcccactaacgaaacagacgataccgcaggcaataaacctaattatcaacggaaacccttagtgtcattcaaagaagatccaacacctagtgataatccctttagtaaattgtataaggaaacaatcgaaacattcgataataacgaagaagaatcatcatactcatacgaagagataaacgatcagactaacgataatataaccgctagactagatagaatagacgaaaaactatctgaaatactaggtatgttacacacactagtagtcgcatctgccggacctacaagtgctagagatgggataagggatgcaatggtagggttaagggaagaaatgatagagaaaattagaaccgaagcattaatgactaacgatagactcgaagcaatggctagacttagaaacgaagaatccgaaaagatggcaaaagatacatctgacgaagtgtcacttaatcctactagcgaaaaattgaataatctattagagggaaacgatagtgataacgatctatcactcgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggatcaacatatacagctgcagtccaatataacgtactcgaaaaagacgacgatcccgctagcctaacaatatgggtcccaatgtttcaatctagtatgcccgctgatctattaatcaaagaactagctaacgttaacatactagtcaaacaaattagtacacctaagggaccctcacttagagtgatgattaatagtagatccgcagtcctagcacaaatgcctagtaagtttacaatatgtgctaacgtaagcttagacgaacgatcaaaactagcatacgatgtgacaacaccatgcgaaatcaaagcatgttcattgacatgtcttaaatcaaagaatatgctaacaacagtcaaagatctaacaatgaaaacacttaatcccacacacgatataatcgcactatgcgaattcgaaaatatagtgactagtaagaaagtgataatccctacataccttagatcaatatccgttagaaataaggatctgaatacactcgaaaatataacaacaaccgaattcaaaaacgctataactaacgctaagataatcccttactccggactattgttagtgataaccgtaaccgataataagggagcattcaaatacataaaaccccaatcccaatttatagtcgatttaggcgcatacttagaaaaagaatcaatctattacgttacaactaattggaaacataccgctactagattcgcaatcaaacctatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctctagcaaattttggccttactttacactaatacacatgataactacaatcatatccctattaatcataatctcaattatgatcgcaatccttaacaaactatgtgagtataacgtattccataacaaaacattcgaattgccaagagctcgagtgaatacctgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 21 - DNA, 15111 bp, respiratory syncytial virus
acgggaaaaaatgcgtacaacaaacttgcataaaccaaaaaaatggggcaaataagaatttgataagtaccacttaaatttaactcccttggttagagatgggcagcaattcattgagtatgataaaagtcagattgcaaaatctattcgataatgacgaagtggcactattaaaaattacatgttataccgataaattgatacatctaactaatgcattagctaaagctgtaatacatacaattaaacttaatggaatagtgtttgtacatgtaattacatctagtgatatatgccctaataataatatcgtagtcaagtctaattttacaacaatgccagtgttacaaaatggcggatatatttgggaaatgatggaattgacacattgctcacaacctaatggtctattagacgataattgcgaaattaaatttagtaagaaattatccgatagtacaatgactaattatatgaatcaattatccgaattgttaggtttcgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgattaccgatatgagaccgttgtcacttgagacaattataactagcctaactagagatataataacacataaatttatatatctgattaatcacgaatgcatcgtgaggaaattggacgaaagacaggccacatttacattcttagtcaattacgaaatgaaactattgcataaggtaggctcaactaagtataagaaatataccgaatataacactaaatacggaacattcccaatgcctatattcataaatcacgacgggtttctcgaatgcataggcataaaacctacaaaacatacacccataatctataaatacgatcttaacccataaatttcaacacaatattcacacaatctaaaacaacaactctatgcataactatactccatagtccagatggagcctgaaaattatagtaatttaaaacttaaggagagatataagatagaagatggggcaaatacagccatggctcttagcaaagtcaagttgaatgatacattgaataaagatcaattactatctagctcgaaatatactatccaacggtctacaggcgattcaatagatacacctaattacgatgtgcaaaaacatattaataaattgtgtggtatgttattgattaccgaagacgcaaatcataaatttacagggttaatcggtatgttatacgctatgtctagattaggtagggaagatacaattaaaatacttagagacgcaggatatcacgttaaagctaacggagtagacgtaactacacatagacaggatattaacggtaaggaaatgaaattcgaagtgttaacactcgctagcttaactaccgaaatacaaattaatatcgaaatcgaatcacgtaaatcttataagaaaatgcttaaagaaatgggcgaagtcgcacccgaatatagacacgatagtcccgattgtggtatgattatactatgtatagccgcattagtgataactaagttggccgcaggcgatagatccggattaaccgcagtgatacgtagagcgaataacgtacttaaaaacgaaatgaaacggtataagggtctattaccaaaagatatagcgaatagtttttacgaagtattcgaaaaacatccacattttatagacgtttttgtgcatttcggaatcgcacaatctagtactagaggaggatctagggttgagggtatattcgcaggattgtttatgaacgcatacggagcaggtcaagtcatgcttagatggggagtactcgcaaaatccgttaaaaatattatgttaggacacgctagcgtacaagccgaaatggaacaagtcgttgaggtatacgaatacgcacaaaaattaggtggagaagcaggattttatcatatactgaataatcctaaagctagtctattaagcttaacacaatttccacatttttctagcgtagtgttaggtaacgcagctggcctaggcataatgggcgaatataggggtacacctagaaatcaggatctatatgacgcagctaaagcatacgctgaacaattgaaagagaatggagtgataaattattccgtactcgatctaacagccgaagagttggaggcaattaaacatcaattgaatccgaaagataatgacgttgagttgtgagttaataaaaaatggggcaaataaatcatcatggaaaagtttgctcctgaattccatggagaagacgcaaataatagggcaacaaaattcttagagtcaatcaagggtaattttacaagtccaaaagatccaaagaagaaagatagtataataagcgtaaactcaattgatatcgaggtgacaaaggaatcacctataacatctaatagtacaataataaatcccactaacgaaacagacgataccgcaggcaataaacctaattatcaacggaaacccttagtgtcattcaaagaagatccaacacctagtgataatccctttagtaaattgtataaggaaacaatcgaaacattcgataataacgaagaagaatcatcatactcatacgaagagataaacgatcagactaacgataatataaccgctagactagatagaatagacgaaaaactatctgaaatactaggtatgttacacacactagtagtcgcatctgccggacctacaagtgctagagatgggataagggatgcaatggtagggttaagggaagaaatgatagagaaaattagaaccgaagcattaatgactaacgatagactcgaagcaatggctagacttagaaacgaagaatccgaaaagatggcaaaagatacatctgacgaagtgtcacttaatcctactagcgaaaaattgaataatctattagagggaaacgatagtgataacgatctatcactcgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggatcaacatatacagctgcagtccaatataacgtactcgaaaaagacgacgatcccgctagcctaacaatatgggtcccaatgtttcaatctagtatgcccgctgatctattaatcaaagaactagctaacgttaacatactagtcaaacaaattagtacacctaagggaccctcacttagagtgatgattaatagtagatccgcagtcctagcacaaatgcctagtaagtttacaatatgtgctaacgtaagcttagacgaacgatcaaaactagcatacgatgtgacaacaccatgcgaaatcaaagcatgttcattgacatgtcttaaatcaaagaatatgctaacaacagtcaaagatctaacaatgaaaacacttaatcccacacacgatataatcgcactatgcgaattcgaaaatatagtgactagtaagaaagtgataatccctacataccttagatcaatatccgttagaaataaggatctgaatacactcgaaaatataacaacaaccgaattcaaaaacgctataactaacgctaagataatcccttactccggactattgttagtgataaccgtaaccgataataagggagcattcaaatacataaaaccccaatcccaatttatagtcgatttaggcgcatacttagaaaaagaatcaatctattacgttacaactaattggaaacataccgctactagattcgcaatcaaacctatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctctagcaaattttggccttactttacactaatacacatgataactacaatcatatccctattaatcataatctcaattatgatcgcaatccttaacaaactatgtgagtataacgtattccataacaaaacattcgaattgccaagagctcgagtgaatacctgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagccattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 22 - DNA, 15111 bp, respiratory syncytial virus
acgggaaaaaatgcgtacaacaaacttgcataaaccaaaaaaatggggcaaataagaatttgataagtaccacttaaatttaactcccttggttagagatgggcagcaattcattgagtatgataaaagtcagattgcaaaatctattcgataatgacgaagtggcactattaaaaattacatgttataccgataaattgatacatctaactaatgcattagctaaagctgtaatacatacaattaaacttaatggaatagtgtttgtacatgtaattacatctagtgatatatgccctaataataatatcgtagtcaagtctaattttacaacaatgccagtgttacaaaatggcggatatatttgggaaatgatggaattgacacattgctcacaacctaatggtctattagacgataattgcgaaattaaatttagtaagaaattatccgatagtacaatgactaattatatgaatcaattatccgaattgttaggtttcgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgattaccgatatgagaccgttgtcacttgagacaattataactagcctaactagagatataataacacataaatttatatatctgattaatcacgaatgcatcgtgaggaaattggacgaaagacaggccacatttacattcttagtcaattacgaaatgaaactattgcataaggtaggctcaactaagtataagaaatataccgaatataacactaaatacggaacattcccaatgcctatattcataaatcacgacgggtttctcgaatgcataggcataaaacctacaaaacatacacccataatctataaatacgatcttaacccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacattgaataaagatcaattactatctagctcgaaatatactatccaacggtctacaggcgattcaatagatacacctaattacgatgtgcaaaaacatattaataaattgtgtggtatgttattgattaccgaagacgcaaatcataaatttacagggttaatcggtatgttatacgctatgtctagattaggtagggaagatacaattaaaatacttagagacgcaggatatcacgttaaagctaacggagtagacgtaactacacatagacaggatattaacggtaaggaaatgaaattcgaagtgttaacactcgctagcttaactaccgaaatacaaattaatatcgaaatcgaatcacgtaaatcttataagaaaatgcttaaagaaatgggcgaagtcgcacccgaatatagacacgatagtcccgattgtggtatgattatactatgtatagccgcattagtgataactaagttggccgcaggcgatagatccggattaaccgcagtgatacgtagagcgaataacgtacttaaaaacgaaatgaaacggtataagggtctattaccaaaagatatagcgaatagtttttacgaagtattcgaaaaacatccacattttatagacgtttttgtgcatttcggaatcgcacaatctagtactagaggaggatctagggttgagggtatattcgcaggattgtttatgaacgcatacggagcaggtcaagtcatgcttagatggggagtactcgcaaaatccgttaaaaatattatgttaggacacgctagcgtacaagccgaaatggaacaagtcgttgaggtatacgaatacgcacaaaaattaggtggagaagcaggattttatcatatactgaataatcctaaagctagtctattaagcttaacacaatttccacatttttctagcgtagtgttaggtaacgcagctggcctaggcataatgggcgaatataggggtacacctagaaatcaggatctatatgacgcagctaaagcatacgctgaacaattgaaagagaatggagtgataaattattccgtactcgatctaacagccgaagagttggaggcaattaaacatcaattgaatccgaaagataatgacgttgagttgtgagttaataaaaaatggggcaaataaatcatcatggaaaagtttgctcctgaattccatggagaagacgcaaataatagggcaacaaaattcttagagtcaatcaagggtaaggttacaagtccaaaagatccaaagaagaaagatagtataataagcgtaaactcaattgatatcgaggtgacaaaggaatcacctataacatctaatagtacaataataaatcccactaacgaaacagacgataccgcaggcaataaacctaattatcaacggaaacccttagtgtcattcaaagaagatccaacacctagtgataatccctttagtaaattgtataaggaaacaatcgaaacattcgataataacgaagaagaatcatcatactcatacgaagagataaacgatcagactaacgataatataaccgctagactagatagaatagacgaaaaactatctgaaatactaggtatgttacacacactagtagtcgcatctgccggacctacaagtgctagagatgggataagggatgcaatggtagggttaagggaagaaatgatagagaaaattagaaccgaagcattaatgactaacgatagactcgaagcaatggctagacttagaaacgaagaatccgaaaagatggcaaaagatacatctgacgaagtgtcacttaatcctactagcgaaaaattgaataatctattagagggaaacgatagtgataacgatctatcactcgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggatcaacatatacagctgcagtccaatataacgtactcgaaaaagacgacgatcccgctagcctaacaatatgggtcccaatgtttcaatctagtatgcccgctgatctattaatcaaagaactagctaacgttaacatactagtcaaacaaattagtacacctaagggaccctcacttagagtgatgattaatagtagatccgcagtcctagcacaaatgcctagtaagtttacaatatgtgctaacgtaagcttagacgaacgatcaaaactagcatacgatgtgacaacaccatgcgaaatcaaagcatgttcattgacatgtcttaaatcaaagaatatgctaacaacagtcaaagatctaacaatgaaaacacttaatcccacacacgatataatcgcactatgcgaattcgaaaatatagtgactagtaagaaagtgataatccctacataccttagatcaatatccgttagaaataaggatctgaatacactcgaaaatataacaacaaccgaattcaaaaacgctataactaacgctaagataatcccttactccggactattgttagtgataaccgtaaccgataataagggagcattcaaatacataaaaccccaatcccaatttatagtcgatttaggcgcatacttagaaaaagaatcaatctattacgttacaactaattggaaacataccgctactagattcgcaatcaaacctatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctctagcaaattttggccttactttacactaatacacatgataactacaatcatatccctattaatcataatctcaattatgatcgcaatccttaacaaactatgtgagtataacgtattccataacaaaacattcgaattgccaagagctcgagtgaatacctgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactatcatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 23 - DNA, 15111 bp, respiratory syncytial virus
acgggaaaaaatgcgtacaacaaacttgcataaaccaaaaaaatggggcaaataagaatttgataagtaccacttaaatttaactcccttggttagagatgggcagcaattcattgagtatgataaaagtcagattgcaaaatctattcgataatgacgaagtggcactattaaaaattacatgttataccgataaattgatacatctaactaatgcattagctaaagctgtaatacatacaattaaacttaatggaatagtgtttgtacatgtaattacatctagtgatatatgccctaataataatatcgtagtcaagtctaattttacaacaatgccagtgttacaaaatggcggatatatttgggaaatgatggaattgacacattgctcacaacctaatggtctattagacgataattgcgaaattaaatttagtaagaaattatccgatagtacaatgactaattatatgaatcaattatccgaattgttaggtttcgatcttaatccataaattataattaatatcaactagcaaatcaatgtcactaacaccattagttaatataaaacttaacagaagacaaaaatggggcaaataaatcaattcagccaacccaaccatggacacaacccacaatgataatacaccacaaagactgatgattaccgatatgagaccgttgtcacttgagacaattataactagcctaactagagatataataacacataaatttatatatctgattaatcacgaatgcatcgtgaggaaattggacgaaagacaggccacatttacattcttagtcaattacgaaatgaaactattgcataaggtaggctcaactaagtataagaaatataccgaatataacactaaatacggaacattcccaatgcctatattcataaatcacgacgggtttctcgaatgcataggcataaaacctacaaaacatacacccataatctataaatacgatcttaacccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacattgaataaagatcaattactatctagctcgaaatatactatccaacggtctacaggcgattcaatagatacacctaattacgatgtgcaaaaacatattaataaattgtgtggtatgttattgattaccgaagacgcaaatcataaatttacagggttaatcggtatgttatacgctatgtctagattaggtagggaagatacaattaaaatacttagagacgcaggatatcacgttaaagctaacggagtagacgtaactacacatagacaggatattaacggtaaggaaatgaaattcgaagtgttaacactcgctagcttaactaccgaaatacaaattaatatcgaaatcgaatcacgtaaatcttataagaaaatgcttaaagaaatgggcgaagtcgcacccgaatatagacacgatagtcccgattgtggtatgattatactatgtatagccgcattagtgataactaagttggccgcaggcgatagatccggattaaccgcagtgatacgtagagcgaataacgtacttaaaaacgaaatgaaacggtataagggtctattaccaaaagatatagcgaatagtttttacgaagtattcgaaaaacatccacattttatagacgtttttgtgcatttcggaatcgcacaatctagtactagaggaggatctagggttgagggtatattcgcaggattgtttatgaacgcatacggagcaggtcaagtcatgcttagatggggagtactcgcaaaatccgttaaaaatattatgttaggacacgctagcgtacaagccgaaatggaacaagtcgttgaggtatacgaatacgcacaaaaattaggtggagaagcaggattttatcatatactgaataatcctaaagctagtctattaagcttaacacaatttccacatttttctagcgtagtgttaggtaacgcagctggcctaggcataatgggcgaatataggggtacacctagaaatcaggatctatatgacgcagctaaagcatacgctgaacaattgaaagagaatggagtgataaattattccgtactcgatctaacagccgaagagttggaggcaattaaacatcaattgaatccgaaagataatgacgttgagttgtgagttaataaaaaatggggcaaataaatcatcatggaaaagtttgctcctgaattccatggagaagacgcaaataatagggcaacaaaattcttagagtcaatcaagggtaaggttacaagtccaaaagatccaaagaagaaagatagtataataagcgtaaactcaattgatatcgaggtgacaaaggaatcacctataacatctaatagtacaataataaatcccactaacgaaacagacgataccgcaggcaataaacctaattatcaacggaaacccttagtgtcattcaaagaagatccaacacctagtgataatccctttagtaaattgtataaggaaacaatcgaaacattcgataataacgaagaagaatcatcatactcatacgaagagataaacgatcagactaacgataatataaccgctagactagatagaatagacgaaaaactatctgaaatactaggtatgttacacacactagtagtcgcatctgccggacctacaagtgctagagatgggataagggatgcaatggtagggttaagggaagaaatgatagagaaaattagaaccgaagcattaatgactaacgatagactcgaagcaatggctagacttagaaacgaagaatccgaaaagatggcaaaagatacatctgacgaagtgtcacttaatcctactagcgaaaaattgaataatctattagagggaaacgatagtgataacgatctatcactcgaagatttctgattagttaccaatcttcacatcaacacacaataccaacagaagaccaacaaactaaccaacccaatcatccaaccaaacatccatccgccaatcagccaaacagccaacaaaacaaccagccaatccaaaactaaccacccggaaaaaatctataatatagttacaaaaaaaggaaagggtggggcaaatatggaaacatacgtgaacaagcttcacgaaggatcaacatatacagctgcagtccaatataacgtactcgaaaaagacgacgatcccgctagcctaacaatatgggtcccaatgtttcaatctagtatgcccgctgatctattaatcaaagaactagctaacgttaacatactagtcaaacaaattagtacacctaagggaccctcacttagagtgatgattaatagtagatccgcagtcctagcacaaatgcctagtaagtttacaatatgtgctaacgtaagcttagacgaacgatcaaaactagcatacgatgtgacaacaccatgcgaaatcaaagcatgttcattgacatgtcttaaatcaatgaatatgctaacaacagtcaaagatctaacaatgaaaacacttaatcccacacacgatataatcgcactatgcgaattcgaaaatatagtgactagtaagaaagtgataatccctacataccttagatcaatatccgttagaaataaggatctgaatacactcgaaaatataacaacaaccgaattcaaaaacgctataactaacgctaagataatcccttactccggactattgttagtgataaccgtaaccgataataagggagcattcaaatacataaaaccccaatcccaatttatagtcgatttaggcgcatacttagaaaaagaatcaatctattacgttacaactaattggaaacataccgctactagattcgcaatcaaacctatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctctagcaaattttggccttactttacactaatacacatgataactacaatcatatccctattaatcataatctcaattatgatcgcaatccttaacaaactatgtgagtataacgtattccataacaaaacattcgaattgccaagagctcgagtgaatacctgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactaccatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt

SEQ ID NO: 24 - DNA, 15111 bp, respiratory syncytial virus
acgggaaaaaatgcgtacaacaaacttgcataaaccaaaaaaatggggcaaataagaatttgataagtaccacttaaatttaactcccttggttagagatgggcagcaattcattgagtatgataaaagtcagattgcaaaatctattcgataatgacgaagtggcactattaaaaattacatgttataccgataaattgatacatctaactaatgcattagctaaagctgtaatacatacaattaaacttaatggaatagtgtttgtacatgtaattacatctagtgatatatgccctaataataatatcgtagtcaagtctaattttacaacaatgccagtgttacaaaatggcggatatatttgggaaatgatggaattgacacattgctcacaacctaatggtctattagacgataattgcgaaattaaatttagtaagaaattatccgatagtacaatgactaattatatgaatcaattatccgaattgttaggtttcgatcttaatccataaattataattaatatcaactagcaaatcaatgtcactaacaccattagttaatataaaacttaacagaagacaaaaatggggcaaataaatcaactcagccaacccaaccatggacacaacccacaatgataatacaccacaaagactgatgattaccgatatgagaccgttgtcacttgagacaattataactagcctaactagagatataataacacataaatttatatatctgattaatcacgaatgcatcgtgaggaaattggacgaaagacaggccacatttacattcttagtcaattacgaaatgaaactattgcataaggtaggctcaactaagtataagaaatataccgaatataacactaaatacggaacattcccaatgcctatattcataaatcacgacgggtttctcgaatgcataggcataaaacctacaaaacatacacccataatctataaatacgatcttaacccataaatttcaacacaatattcacacaatctaaaacaacaactctatgcataactatactccatagtccagatggagcctgaaaattatagtaatttaaaacttaaggagagatataagatagaagatggggcaaatacaaccatggctcttagcaaagtcaagttgaatgatacattgaataaagatcaattactatctagctcgaaatatactatccaacggtctacaggcgattcaatagatacacctaattacgatgtgcaaaaacatattaataaattgtgtggtatgttattgattaccgaagacgcaaatcataaatttacagggttaatcggtatgttatacgctatgtctagattaggtagggaagatacaattaaaatacttagagacgcaggatatcacgttaaagctaacggagtagacgtaactacacatagacaggatattaacggtaaggaaatgaaattcgaagtgttaacactcgctagcttaactaccgaaatacaaattaatatcgaaatcgaatcacgtaaatcttataagaaaatgcttaaagaaatgggcgaagtcgcacccgaatatagacacgatagtcccgattgtggtatgattatactatgtatagccgcattagtgataactaagttggccgcaggcgatagatccggattaaccgcagtgatacgtagagcgaataacgtacttaaaaacgaaatgaaacggtataagggtctattaccaaaagatatagcgaatagtttttacgaagtattcgaaaaacatccacattttatagacgtttttgtgcatttcggaatcgcacaatctagtactagaggaggatctagggttgagggtatattcgcaggattgtttatgaacgcatacggagcaggtcaagtcatgcttagatggggagtactcgcaaaatccgttaaaaatattatgttaggacacgctagcgtacaagccgaaatggaacaagtcgttgaggtatacgaatacgcacaaaaattaggtggagaagcaggattttatcatatactgaataatcctaaagctagtctattaagcttaacacaatttccacatttttctagcgtagtgttaggtaacgcagctggcctaggcataatgggcgaatataggggtacacctagaaatcaggatctatatgacgcagctaaagcatacgctgaacaattgaaagagaatggagtgataaattattccgtactcgatctaacagccgaagagttggaggcaattaaacatcaattgaatccgaaagataatgacgttgagttgtgagttaataaaaaatggggtaaataaatcatcatggaaaagtttgctcctgaattccatggagaagacgcaaataatagggcaacaaaattcttagagtcaatcaagggtaaggttacaagtccaaaagatccaaagaagaaagatagtataataagcgtaaactcaattgatatcgaggtgacaaaggaatcacctataacatctaatagtacaataataaatcccactaacgaaacagacgataccgcaggcaataaacctaattatcaacggaaacccttagtgtcattcaaagaagatccaacacctagtgataatccctttagtaaattgtataaggaaacaatcgaaacattcgataataacgaagaagaatcatcatactcatacgaagagataaacgatcagactaacgataatataaccgctagactagatagaatagacgaaaaactatctgaaatactaggtatgttacacacactagtagtcgcatctgccggacctacaagtgctagagatgggataagggatgcaatggtagggttaagggaagaaatgatagagaaaattagaaccgaagcattaatgactaacgatagactcgaagcaatggctagacttagaaacgaagaatccgaaaagatggcaaaagatacatctgacgaagtgtcacttaatcctactagcgaaaaattgaataatctattagagggaaacgatagtgataacgatctatcactcgaagatttctgattagttaccaatcttcacatcaacacacaataccaacagaagaccaacaaactaaccaacccaatcatccaaccaaacatccatccgccaatcagccaaacagccaacaaaacaaccagccagtccaaaactaaccacccggaaaaaatctataatatagttacaaaaaaaggaaagggtggggcaaatatggaaacatacgtgaacaagcttcacgaaggatcaacatatacagctgcagtccaatataacgtactcgaaaaagacgacgatcccgctagcctaacaatatgggtcccaatgtttcaatctagtatgcccgctgatctattaatcaaagaactagctaacgttaacatactagtcaaacaaattagtacacctaagggaccctcacttagagtgatgattaatagtagatccgcagtcctagcacaaatgcctagtaagtttacaatatgtgctaacgtaagcttagacgaacgatcaaaactagcatacgatgtgacaacaccatgcgaaatcaaagcatgttcattgacatgtcttaaatcaatgaatatgctaacaacagtcaaagatctaacaatgaaaacacttaatcccacacacgatataatcgcactatgcgaattcgaaaatatagtgactagtaagaaagtgataatccctacataccttagatcaatatccgttagaaataaggatctgaatacactcgaaaatataacaacaaccgaattcaaaaacgctataactaacgctaagataatcccttactccggactattgttagtgataaccgtaaccgataataagggagcattcaaatacataaaaccccaatcccaatttatagtcgatttaggcgcatacttagaaaaagaatcaatctattacgttacaactaattggaaacataccgctactagattcgcaatcaaacctatggaagattaacctttttcctctacatcagtgtgttaattcatacaaactttctacctacattcttcacttcaccatcacaatcacaaacactctgtggttcaaccaatcaaacaaaacttatctgaagtcccagatcatcccaagtcattgtttatcagatctagtactcaaataagttaataaaaaatatacacatggggcaaataatcattggaggaaatccaactaatcacaatatctgttaacatagacaagtccacacaccatacagaatcaaccaatggaaaatacatccataacaatagaattctctagcaaattttggccttactttacactaatacacatgataactacaatcatatccctattaatcataatctcaattatgatcgcaatccttaacaaactatgtgagtataacgtattccataacaaaacattcgaattgccaagagctcgagtgaatacctgataaagtagttaattaaaaatagtcataacaatgaactaggatatcaagactaacaataacattggggcaaatgcaaacatgtccaaaaacaaggaccaacgcaccgctaagacattagaaaggacctgggacactctcaatcatttattattcatatcatcgtgcttatataagttaaatcttaaatctgtagcacaaatcacattatccattctggcaatgataatctcaacttcacttataattgcagccatcatattcatagcctcggcaaaccacaaagtcacaccaacaactgcaatcatacaagatgcaacaagccagatcaagaacacaaccccaacatacctcacccagaatcctcagcttggaatcagtccctctaatccgtctgaaattacatcacaaatcaccaccatactagcttcaacaacaccaggagtcaagtcaaccctgcaatccacaacagtcaagaccaaaaacacaacaacaactcaaacacaacccagcaagcccaccacaaaacaacgccaaaacaaaccaccaagcaaacccaataatgattttcactttgaagtgttcaactttgtaccctgcagcatatgcagcaacaatccaacctgctgggctatctgcaaaagaataccaaacaaaaaaccaggaaagaaaaccactaccaagcccacaaaaaaaccaaccctcaagacaaccaaaaaagatcccaaacctcaaaccactaaatcaaaggaagtacccaccaccaagcccacagaagagccaaccatcaacaccaccaaaacaaacatcataactacactactcacctccaacaccacaggaaatccagaactcacaagtcaaatggaaaccttccactcaacttcctccgaaggcaatccaagcccttctcaagtctctacaacatccgagtacccatcacaaccttcatctccacccaacacaccacgccagtagttacttaaaaacatattatcacaaaaggccttgaccaacttaaacagaatcaaaataaactctggggcaaataacaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgttttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggcgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaacacaattgcatgccagattaacttaccatctgtaaaaatgaaaactggggcaaatatgtcacgaaggaatccttgcaaatttgaaattcgaggtcattgcttaaatggtaagaggtgtcattttagtcataattattttgaatggccaccccatgcactgcttgtaagacaaaactttatgttaaacagaatacttaagtctatggataaaagtatagataccttatcagaaataagtggagctgcagagttggacagaacagaagagtatgctcttggtgtagttggagtgctagagagttatataggatcaataaacaatataactaaacaatcagcatgtgttgccatgagcaaactcctcactgaactcaatagtgatgatatcaaaaagctgagggacaatgaagagctaaattcacccaagataagagtgtacaatactgtcatatcatatattgaaagcaacaggaaaaacaataaacaaactatccatctgttaaaaagattgccagcagacgtattgaagaaaaccatcaaaaacacattggatatccataagagcataaccatcaacaacccaaaagaatcaactgttagtgatacaaatgaccatgccaaaaataatgatactacctgacaaatatccttgtagtataacttccatactaataacaagtagatgtagagttactatgtataatcaaaagaacacactatatttcaatcaaaacaacccaaataaccatatgtactcaccgaatcaaacattcaatgaaatccattggacctctcaagaattgattgacacaattcaaaattttctacaacatctaggtattattgaggatatatatacaatatatatattagtgtcataacactcaattctaacactcaccacatcgttacattattaattcaaacaattcaagttgtgggacaaaatggatcccattattaatggaaattctgctaatgtttatctaaccgatagttatttaaaaggtgttatctctttctcagagtgtaatgctttaggaagttacatattcaatggtccttatctcaaaaatgattataccaacttaattagtagacaaaatccattaatagaacacatgaatctaaagaaactaaatataacacagtccttaatatctaagtatcataaaggtgaaataaaattagaagaacctacttattttcagtcattacttatgacatacaagagtatgacctcgtcagaacagattgctaccactaatttacttaaaaagataataagaagagctatagaaataagtgatgtcaaagtctatgctatattgaataaactagggcttaaagaaaaggacaagattaaatccaacaatggacaagatgaagacaactcagttattacgaccataatcaaagatgatatactttcagctgttaaagataatcaatctcatcttaaagcagacaaaaatcactctacaaaacaaaaagacacaatcaaaacaacactcttgaagaaattgatgtgttcaatgcaacatcctccatcatggttaatacattggtttaacttatacacaaaattaaacaacatattaacacagtatcgatcaaatgaggtaaaaaaccatgggtttacattgatagataatcaaactcttagtggatttcaatttattttgaaccaatatggttgtatagtttatcataaggaactcaaaagaattactgtgacaacctataatcaattcttgacatggaaagatattagccttagtagattaaatgtttgtttaattacatggattagtaactgcttgaacacattaaataaaagcttaggcttaagatgcggattcaataatgttatcttgacacaactattcctttatggagattgtatactaaagctatttcacaatgaggggttctacataataaaagaggtagagggatttattatgtctctaattttaaatataacagaagaagatcaattcagaaaacgattttataatagtatgctcaacaacatcacagatgctgctaataaagctcagaaaaatctgctatcaagagtatgtcatacattattagataagacagtgtccgataatataataaatggcagatggataattctattaagtaagttccttaaattaattaagcttgcaggtgacaataaccttaacaatctgagtgaactatattttttgttcagaatatttggacacccaatggtagatgaaagacaagccatggatgctgttaaaattaattgcaatgagaccaaattttacttgttaagcagtctgagtatgttaagaggtgcctttatatatagaattataaaagggtttgtaaataattacaacagatggcctactttaagaaatgctattgttttacccttaagatggttaacttactataaactaaacacttatccttctttgttggaacttacagaaagagatttgattgtgttatcaggactacgtttctatcgtgagtttcggttgcctaaaaaagtggatcttgaaatgattataaatgataaagctatatcacctcctaaaaatttgatatggactagtttccctagaaattacatgccatcacacatacaaaactatatagaacatgaaaaattaaaattttccgagagtgataaatcaagaagagtattagagtattatttaagagataacaaattcaatgaatgtgatttatacaactgtgtagttaatcaaagttatctcaacaaccctaatcatgtggtatcattgacaggcaaagaaagagaactcagtgtaggtagaatgtttgcaatgcaaccgggaatgttcagacaggttcaaatattggcagagaaaatgatagctgaaaacattttacaattctttcctgaaagtcttacaagatatggtgatctagaactacaaaaaatattagaactgaaagcaggaataagtaacaaatcaaatcgctacaatgataattacaacaattacattagtaagtgctctatcatcacagatctcagcaaattcaatcaagcatttcgatatgaaacgtcatgtatttgtagtgatgtgctggatgaactgcatggtgtacaatctctattttcctggttacatttaactattcctcatgtcacaataatatgcacatataggcatgcacccccctatataggagatcatattgtagatcttaacaatgtagatgaacaaagtggattatatagatatcacatgggtggcatcgaagggtggtgtcaaaaactatggaccatagaagctatatcactattggatctaatatctctcaaagggaaattctcaattactgctttaattaatggtgacaatcaatcaatagatataagcaaaccaatcagactcatggaaggtcaaactcatgctcaagcagattatttgctagcattaaatagccttaaattactgtataaagagtatgcaggcataggccacaaattaaaaggaactgagacttatatatcacgagatatgcaatttatgagtaaaacaattcaacataacggtgtatattacccagctagtataaagaaagtcctaagagtgggaccgtggataaacactatacttgatgatttcaaagtgagtctagaatctataggtagtttgacacaagaattagaatatagaggtgaaagtctattatgcagtttaatatttagaaatgtatggttatataatcagattgctctacaattaaaaaatcatgcattatgtaacaataaactatatttggacatattaaaggttctgaaacacttaaaaaccttttttaatcttgataatattgatacagcattaacattgtatatgaatttacccatgttatttggtggtggtgatcccaacttgttatatcgaagtttctatagaagaactcctgacttcctcacagaggctatagttcactctgtgttcatacttagttattatacaaaccatgacttaaaagataaacttcaagatctgtcagatgatagattgaataagttcttaacatgcataatcacgtttgacaaaaaccctaatgctgaattcgtaacattgatgagagatcctcaagctttagggtctgagagacaagctaaaattactagcgaaatcaatagactggcagttacagaggttttgagtacagctccaaacaaaatattctccaaaagtgcacaacattatactactacagagatagatctaaatgatattatgcaaaatatagaacctacatatcctcatgggctaagagttgtttatgaaagtttacccttttataaagcagagaaaatagtaaatcttatatcaggtacaaaatctataactaacatactggaaaaaacttctgccatagacttaacagatattgatagagccactgagatgatgaggaaaaacataactttgcttataaggatacttccattggattgtaacagagataaaagagagatattgagtatggaaaacctaagtattactgaattaagcaaatatgttagggaaagatcttggtctttatccaatatagttggtgttacatcacccagtatcatgtatacaatggacatcaaatatactacaagcactatatctagtggcataattatagagaaatataatgttaacagtttaacacgtggtgagagaggacccactaaaccatgggttggttcatctacacaagagaaaaaaacaatgccagtttataatagacaagtcttaaccaaaaaacagagagatcaaatagatctattagcaaaattggattgggtgtatgcatctatagataacaaggatgaattcatggaagaactcagcataggaacccttgggttaacatatgaaaaggccaagaaattatttccacaatatttaagtgtcaattatttgcatcgccttacagtcagtagtagaccatgtgaattccctgcatcaataccagcttatagaacaacaaattatcactttgacactagccctattaatcgcatattaacagaaaagtatggtgatgaagatattgacatagtattccaaaactgtataagctttggccttagtttaatgtcagtagtagaacaatttactaatgtatgtcctaacagaattattctcatacctaagcttaatgagatacatttgatgaaacctcccatattcacaggtgatgttgatattcacaagttaaaacaagtgatacaaaaacagcatatgtttttaccagacaaaataagtttgactcaatatgtggaattattcttaagtaataaaacactcaaatctggatctcatgttaattctaatttaatattggcacataaaatatctgactattttcataatacttacattttaagtactaatttagctggacattggattctgattatacaacttatgaaagattctaaaggtatttttgaaaaagattggggagagggatatataactgatcatatgtttattaatttgaaagttttcttcaatgcttataagacctatctcttgtgttttcataaaggttatggcaaagcaaagctggagtgtgatatgaacacttcagatcttctatgtgtattggaattaatagacagtagttattggaagtctatgtctaaggtatttttagaacaaaaagttatcaaatacattcttagccaagatgcaagtttacatagagtaaaaggatgtcatagcttcaaattatggtttcttaaacgtcttaatgtagcagaattcacagtttgcccttgggttgttaacatagattatcatccaacacatatgaaagcaatattaacttatatagatcttgttagaatgggattgataaatatagatagaatacacattaaaaataaacacaaattcaatgatgaattttatacttctaatctcttctacattaattataacttctcagataatactcatctattaactaaacatataaggattgctaattctgaattagaaaataattacaacaaattatatcatcctacaccagaaaccctagagaatatactagccaatccgattaaaagtaatgacaaaaagacactgaatgactattgtataggtaaaaatgttgactcaataatgttaccattgttatctaataagaagcttattaaatcgtctgcaatgattagaaccaattacagcaaacaagatttgtataatttattccctatggttgtgattgatagaattatagatcattcaggcaatacagccaaatccaaccaactttacactactacttcccaccaaatatccttagtgcacaatagcacatcactttactgcatgcttccttggcatcatattaatagattcaattttgtatttagttctacaggttgtaaaattagtatagagtatattttaaaagatcttaaaattaaagatcccaattgtatagcattcataggtgaaggagcagggaatttattattgcgtacagtagtggaacttcatcctgacataagatatatttacagaagtctgaaagattgcaatgatcatagtttacctattgagtttttaaggctgtacaatggacatatcaacattgattatggtgaaaatttgaccattcctgctacagatgcaaccaacaacattcattggtcttatttacatataaagtttgctgaacctatcagtctttttgtctgtgatgccgaattgtctgtaacagtcaactggagtaaaattataatagaatggagcaagcatgtaagaaagtgcaagtactgttcctcagttaataaatgtatgttaatagtaaaatatcatgctcaagatgatattgatttcaaattagacaatataactatattaaaaacttatgtatgcttaggcagtaagttaaagggatcggaggtttacttagtccttacaataggtcctgcgaatatattcccagtatttaatgtagtacaaaatgctaaattgatactatcaagaaccaaaaatttcatcatgcctaagaaagctgataaagagtctattgatgcaaatattaaaagtttgataccctttctttgttaccctataacaaaaaaaggaattaatactgcattgtcaaaactaaagagtgttgttagtggagatatactaccatattctatagctggacgtaatgaagttttcagcaataaacttataaatcataagcatatgaacatcttaaaatggttcaatcatgttttaaatttcagatcaacagaactaaactataaccatttatatatggtagaatctacatatccttacctaagtgaattgttaaacagcttgacaaccaatgaacttaaaaaactgattaaaatcacaggtagtctgttatacaactttcataatgaataatgaataaagatcttataataaaaattcccatagctatacactaacactgtattcaattatagttattaaaaattaaaaatcatataattttttaaataacttttagtgaactaatcctaaagttatcattttaatcttggaggaataaatttaaaccctaatctaattggtttatatgtgtattaactaaattacgagatattagtttttgacactttttttctcgt